(19) United States
(12) Patent Application Publication
(10) Pub. No.: US 2026/0132206 A1
(43) Pub. Date: May 14, 2026

(54) METHODS OF TREATING CHRONIC THYROID EYE DISEASE

(71) Applicant: Viridian Therapeutics, Inc., Waltham, MA (US)

(72) Inventor: Thomas CIULLA, Waltham, MA (US)

(73) Assignee: Viridian Therapeutics, Inc., Waltham, MA (US)

(21) Appl. No.: 19/442,626

(22) Filed: Jan. 7, 2026

Related U.S. Application Data
(63) Continuation of application No. PCT/US2024/036800, filed on Jul. 3, 2024.
(60) Provisional application No. 63/512,468, filed on Jul. 7, 2023.

(51) Int. Cl. C07K 16/28; A61K 39/00; A61P 27/02

(52) U.S. Cl. CPC C07K 16/2863; A61P 27/02; A61K 2039/505

(57) ABSTRACT

Methods of treating patients with chronic thyroid eye disease are provided for herein, such as by administering antibodies and compositions that bind to and/or antagonize IGF-1R.

### CROSS REFERENCE TO RELATED APPLICATIONS

The present application is a Continuation of International Application No. PCT/US2024/036800, filed Jul. 3, 2024, which claims the benefit of priority to U.S. Provisional Patent Application No. 63/512,468, filed on Jul. 7, 2023, all of which are hereby incorporated by reference in their entireties.

### INCORPORATION BY REFERENCE OF SEQUENCE LISTING

The instant application contains a Sequence Listing which has been submitted electronically in XML file format and is hereby incorporated by reference in its entirety. Said XML copy, created on Jan. 6, 2026, is named VRD-016US1_SL and is 41,108 bytes in size.

### BACKGROUND

Thyroid Eye Disease (TED) is an autoimmune condition most commonly associated with Graves' disease and hyperthyroidism but can also be found in patients who are euthyroid or hypothyroid. The disease course has historically been said to transition from a first active and progressive phase (“active TED”), which is characterized by inflammation of orbital and external periorbital tissues, to a more stabilized and fibrotic phase. Active TED can be characterized by local inflammation of conjunctivae, superficial vasculature, orbital fat, lids, and extraocular muscles. The second phase of TED can be characterized by a dampened autoimmune inflammation of the first phase, leaving sequelae of expanded, fibrotic orbital tissues and dysfunctional, tethered extraocular muscles, though evidence exists that these TED patients may also exhibit an underlying inflammatory component. Accordingly, the onset of this non-active phase may be indicated by the duration, severity, and/or nature of disease symptoms. In view of the different natures of the two disease phases, there remains a need for effective therapies for TED.

### SUMMARY

The disclosure relates generally to IGF-1R antibodies, and antigen binding fragments thereof, including any forms, variants, or derivatives thereof, and uses thereof. Certain IGF-1R antibodies and antigen-binding fragments inhibit IGF-1R function or block the biological functions of IGF-I mediated IGF-1R signaling. Additionally, the invention generally relates to methods for treating thyroid-associated ophthalmopathy (TAO), also known as thyroid eye disease (TED), Graves' ophthalmopathy or orbitopathy (GO), thyrotoxic exophthalmos, dysthyroid ophthalmopathy, autoimmune associated eye disorders associated with IGF-1R signaling, inflammatory orbital disorder associated with IGF-1R signaling, and other thyroid eye disorders associated with IGF-1R signaling, including chronic TED.

In one aspect, the invention features a method of treating a patient suffering from chronic thyroid eye disease (TED), the method comprising administering a pharmaceutical composition comprising an anti-IGF-1R antibody, wherein the antibody comprises a heavy chain and a light chain, wherein the heavy chain comprises a HCDR1, a HCDR2, and a HCDR3 of SEQ ID NO: 10 and the light chain comprises a LCDR1, a LCDR2, and a LCDR3 of SEQ ID NO: 11. In some embodiments, the anti-IGF-1R antibody is administered at a therapeutically effective dosing regimen.

In another aspect, the invention features a method of treating a patient suffering from thyroid eye disease (TED), the method comprising administering a pharmaceutical composition comprising an anti-IGF-1R antibody, wherein the antibody comprises a heavy chain and a light chain, wherein the heavy chain comprises a HCDR1 of SEQ ID NO: 7, a HCDR2 of SEQ ID NO: 8, and a HCDR3 of SEQ ID NO: 9 and the light chain comprises a LCDR1 of SEQ ID NO: 4, a LCDR2 of SEQ ID NO: 5, and a LCDR3 of SEQ ID NO: 6, wherein the patient, prior to treatment, has had one or more symptoms of thyroid eye disease for at least 12 months. In some embodiments, the anti-IGF-1R antibody is administered at a therapeutically effective dosing regimen.

In some embodiments, a patient, prior to treatment an anti-IGF-1R antibody, has had one or more symptoms of thyroid eye disease for at least 12 months. In some embodiments, a patient, prior to treatment with an anti-IGF-1R antibody, has had one or more symptoms of thyroid eye disease for at least 15 months. In some embodiments, a patient, prior to treatment with an anti-IGF-1R antibody, has had one or more symptoms of thyroid eye disease associated with a Clinical Activity Score (CAS) of ≥4, where such symptom(s) have lessened in severity over time. In some embodiments, a patient, prior to treatment with an anti-IGF-1R antibody, has had one or more symptoms of thyroid eye disease that have reached a plateau, e.g., a static some embodiments, a patient, prior to treatment with an anti-IGF-1R antibody, has had one or more symptoms of thyroid eye disease associated with a Clinical Activity Score (CAS) of ≥4, where such symptom(s) have lessened in severity over time. In some embodiments, a patient, prior to treatment with an anti-IGF-1R antibody, has had one or more symptoms of thyroid eye disease that have reached a plateau, e.g., a static plateau.

In some embodiments, a patient suffers one or more symptoms of TED selected from the group consisting of lid retraction greater than 2 mm, exophthalmos (proptosis) of greater than or equal to 3 mm, Clinical Activity Score (CAS) from about 0 to about 7, and inconstant or constant diplopia.

In some embodiments, the exophthalmos is greater than or equal to 3 mm above the normal range for their race and gender.

In some embodiments, a patient, prior to treatment with an anti-IGF-1R antibody, had a CAS of or greater than 0, 1, 2, 3, or 4.

In some embodiments, a patient, prior to treatment with an anti-IGF-1R antibody, had a CAS of greater than 2.

In some embodiments, a patient, prior to treatment with an anti-IGF-1R antibody, had a CAS less than 2.

In some embodiments, a patient, prior to treatment with an anti-IGF-1R antibody, had a CAS of 2-4.

In some embodiments, patient, prior to treatment with an anti-IGF-1R antibody, has had one or more symptoms of thyroid eye disease for at least 12 months. In some embodiments, patient, prior to treatment with an anti-IGF-1R antibody, has had one or more symptoms of thyroid eye disease for at least 15 months.

In another aspect, the invention features a method of treating a patient suffering from inactive thyroid eye disease (TED), the method comprising administering a pharmaceutical composition comprising an anti-IGF-1R antibody, wherein the antibody comprises a heavy chain and a light chain, wherein the heavy chain comprises a HCDR1 of SEQ ID NO: 7, a HCDR2 of SEQ ID NO: 8, and a HCDR3 of SEQ ID NO: 9 and the light chain comprises a LCDR1 of SEQ ID NO: 4, a LCDR2 of SEQ ID NO: 5 and a LCDR3 of SEQ ID NO: 6. In embodiments, the anti-IGF-1R antibody is administered at a therapeutically effective dosing regimen.

In another aspect, the invention features a method of treating a patient suffering from thyroid eye disease (TED), the method comprising administering a pharmaceutical composition comprising an anti-IGF-1R antibody at a therapeutically effective dosing regimen, wherein the antibody comprises a heavy chain and a light chain, wherein the heavy chain comprises a HCDR1 of SEQ ID NO: 7, a HCDR2 of SEQ ID NO: 8, and a HCDR3 of SEQ ID NO: 9 and the light chain comprises a LCDR1 of SEQ ID NO: 4, a LCDR2 of SEQ ID NO: 5 and a LCDR3 of SEQ ID NO: 6, wherein the patient, prior to treatment, had a Clinical Activity Score (CAS) of 2 or less.

In some embodiments, a patient exhibits fibrosis.

In another aspect, the invention features a method of treating fibrosis associated with thyroid eye disease (TED), the method comprising administering to a patient in need of treatment a pharmaceutical composition comprising an anti-IGF-1R antibody at a therapeutically effective dosing regimen, wherein the antibody comprises a heavy chain and a light chain, wherein the heavy chain comprises a HCDR1 of SEQ ID NO: 7, a HCDR2 of SEQ ID NO: 8, and a HCDR3 of SEQ ID NO: 9 and the light chain comprises a LCDR1 of SEQ ID NO: 4, a LCDR2 of SEQ ID NO: 5 and a LCDR3 of SEQ ID NO: 6.

In some embodiments, a patient, prior to treatment with an anti-IGF-1R antibody, had a Clinical Activity Score (CAS) of 0 or 1.

In some embodiments, a patient has one or more symptoms of TED selected from the group consisting of lid retraction greater than 2 mm, exophthalmos (proptosis) of greater than or equal to 3 mm above the normal range for their race and gender, and inconstant or constant diplopia.

In some embodiments, a patient, prior to treatment with an anti-IGF-1R antibody, has had one or more symptoms of thyroid eye disease for at least 2 years. In some embodiments, a patient, prior to treatment with an anti-IGF-1R antibody, has had one or more symptoms of thyroid eye disease for at least 3 years.

In some embodiments, a therapeutically effective dosing regimen comprises administering to the patient a first dose of about 3.0 mg/kg to about 20 mg/kg.

In some embodiments, a first dose is about 3.0 mg/kg, about 5.0 mg/kg, about 10 mg/kg, or about 20 mg/kg.

In some embodiments, a therapeutically effective dosing regimen comprises administering to the patient a subsequent dose.

In some embodiments, a subsequent dose is about 3.0 mg/kg to about 20 mg/kg.

In some embodiments, a subsequent dose is about 3.0 mg/kg, about 5.0 mg/kg, about 10 mg/kg, or about 20 mg/kg.

In some embodiments, a subsequent dose is administered to the patient once every two weeks, once every three weeks, once every four weeks, once every month, once every five weeks, or once every six weeks.

In some embodiments, a subsequent dose is administered to the patient once every three weeks.

In some embodiments, a patient is administered at least 3, 4, 5, 6, 7, 8, 9 or 10 subsequent doses.

In some embodiments, a patient is administered at least 4 subsequent doses.

In some embodiments, a patient is administered at least 5 subsequent doses.

In some embodiments, a patient is administered with a dosing regimen comprising at least 8 subsequent doses.

In some embodiments, administration of the dosing regimen results in reduction of the proptosis by about 1 to about 3 mm, about 1 to about 2 mm, about 2 to about 3 mm from baseline within 6 weeks of the first dose as measured by exophthalmometry or by MRI/CT.

In some embodiments, proptosis is reduced by about 2-3 mm from baseline within 6 weeks of the first dose as measured by exophthalmometry or by MRI/CT.

In some embodiments, a patient does not have hearing impairment, ototoxic changes in audiometry, or hyperglycemic levels in the patient during the treatment.

In some embodiments, a light chain comprises a variable region having the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 2 and a heavy chain comprises a variable region sequence having the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 3. In some embodiments, a light chain comprises a variable region having the amino acid sequence of SEQ ID NO: 2 and a heavy chain comprises a variable region sequence having the amino acid sequence of SEQ ID NO: 3.

In some embodiments, a light chain comprises an amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 11. In some embodiments, a light chain comprises an amino acid sequence of SEQ ID NO: 11.

In some embodiments, a light chain comprises an amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 10. In some embodiments, a heavy chain comprises an amino acid sequence of SEQ ID NO: 10.

In some embodiments, a heavy chain comprises an amino acid sequence of SEQ ID NO: 10 and a light chain comprises an amino acid sequence of SEQ ID NO: 11.

In embodiments of any methods described herein, an anti-IGF-1R antibody includes the anti-IGF-1R antibody and antigen binding fragments thereof, including any forms, variants, or derivatives thereof of the anti-IGF-1R antibody and antigen binding fragments.

In some embodiments, the patient achieves an improvement in one or more parameters selected from: proptosis, CAS, extraocular muscle volume, orbital fat volume, manual measurement of lid retraction, Graves' Orbitopathy-Quality of Life (GO-QoL) combined score, GO-QoL activity subscale, GO-QoL appearance subscale, visual acuity, Gorman Subjective Diplopia Score, and EQ-5D-5L QoL questionnaire.

### BRIEF DESCRIPTION OF THE DRAWING

FIG. 1 illustrates various data from patients with chronic thyroid eye disease treated with anti-IGF-1R antibodies, such as those provided for herein.

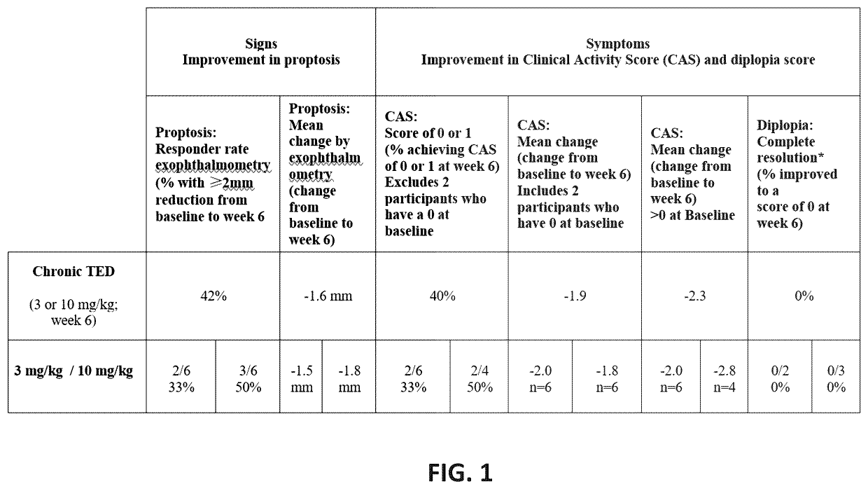

FIG. 2 illustrates an exemplary Phase 3 study design schematic for assessing VRDN-5000 in participants with chronic TED.

### DETAILED DESCRIPTION

Provided herein are antibodies that bind and modulate the activity of IGF-1R. The antibodies can be used, for example, to treat thyroid-associated ophthalmopathy (TAO), also known as thyroid eye disease (TED), Graves' ophthalmopathy or orbitopathy (GO), thyrotoxic exophthalmos, dysthyroid ophthalmopathy, autoimmune associated eye disorders associated with IGF-1R signaling, inflammatory orbital disorder associated with IGF-1R signaling, and other thyroid eye disorders associated with IGF-1R signaling, including chronic TED.

As used herein, “Thyroid-associated Ophthalmopathy” (TAO), “Thyroid Eye Disease” (TED), “Graves' Ophthalmopathy” or “Graves' Orbitopathy” (GO) refer to the same disorder or condition and are used interchangeably. They all refer to the inflammatory orbital pathology associated with some autoimmune thyroid disorders, most commonly with “Graves' Disease” (GD), but sometimes with other diseases, e.g., Hashimoto's thyroiditis.

The terms “proptosis” and “exophthalmos” (also known as exophthalmus, exophthalmia, or exorbitism) refer to the forward projection, displacement, bulging, or protrusion of an organ. As used herein, the terms refer to the forward projection, displacement, bulging, or protrusion of the eye anteriorly out of the orbit. Proptosis and exophthalmos are considered by some of skill in the art to have the same meaning and are often used interchangeably, while others attribute subtle differences to their meanings. Exophthalmos is used by some to refer to severe proptosis; or to refer to endocrine-related proptosis. Yet others use the term exophthalmos when describing proptosis associated with the eye, in, for example, subjects with TAO (TED or GO).

As used herein, the terms “proptosis” and “exophthalmos” are used interchangeably and refer to the forward projection, displacement, bulging, or protrusion of the eye anteriorly out of the orbit. Owing to the rigid bony structure of the orbit with only anterior opening for expansion, any increase in orbital soft tissue contents taking place from the side or from behind will displace the eyeball forward. Proptosis or exophthalmos can be the result of a several disease processes including infections, inflammations, tumors, trauma, metastases, endocrine lesions, vascular diseases & extra orbital lesions. TAO (TED or GO) is currently recognized as the most common cause of proptosis in adults. Exophthalmos can be either bilateral, as is often seen in TAO (TED or GO), or unilateral (as is often seen in an orbital tumor).

Measurement of the degree of exophthalmos can be performed using, for example, an exophthalmometer, an instrument used for measuring the degree of forward displacement of the eye. The device allows measurement of the forward distance of the lateral orbital rim to the front of the cornea. Computed tomography (CT) scanning and Magnetic resonance imaging (MRI) may also be used in evaluating the degree of exophthalmos or proptosis. CT scanning is an excellent imaging modality for the diagnosis of TAO. In addition to allowing visualization of the enlarged extraocular muscles, CT scans provide the surgeon or clinician with depictions of the bony anatomy of the orbit when an orbital decompression is required. MRI, with its multi-planar and inherent contrast capabilities, provides excellent imaging of the orbital contents without the radiation exposure associated with CT scan studies. MRI provides better imaging of the optic nerve, orbital fat, and extraocular muscle, but CT scans provide better views of the bony architecture of the orbit. Orbital ultrasonography can also be used for the diagnosis and evaluation of TAO, because it can be performed quickly and with a high degree of confidence. High reflectivity and enlargement of the extraocular muscles are assessed easily, and serial ultrasonographic examinations can also be used to assess progression or stability of the ophthalmopathy. Based on the technologies currently available, or that will become available in the future, one of skill in the art would be capable of determining the best modality for diagnosing and evaluating the extent of proptosis or exophthalmos.

As used herein, the term “antibody” refers to any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic antibody.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g., fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

A “Fab fragment” is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}1 and C_{H}2 domains of an antibody. The two heavy chain fragments are held together by two or more disulfide bonds and by hydrophobic interactions of the C_{H}3 domains.

In some embodiments, the antibodies, or antigen fragments herein, comprise a Fc region. In some embodiments, the Fc region comprises a mutation that extends the half-life of the antibody when linked to the Fc region. In some embodiments, the Fc region comprises a S228P, L235E, M252Y, S254T, T256E, M428L, N434S, L234F, P331S mutation, or any combination thereof. In some embodiments, the Fc region comprises a M252Y, S254T, and T256E mutations. In some embodiments, the Fc region comprises a S228P and a L235E mutation. In some embodiments, the antibody comprises a L234F, L235E, and P331S mutation. In some embodiments, the Fc region comprises M252Y, S254T, T256E, S228P and L235E mutations. In some embodiments, the Fc region comprises S228P, L235E, M428L, and N434S mutations. In some embodiments, the Fc region comprises the M428L and N434S mutations. In some embodiments, the Fc region comprises the L234F, L235E, P331S, M252Y, S254T, and T256E mutations. In some embodiments, the Fc region comprises a S228P, L235E, M252Y, S254T, T256E, M428L, N434S, L234F, P331S, T250Q, L309D, Q311H, N434Y, Q311R, M428E, N434W mutation, or any combination thereof. In some embodiments, the Fc region comprises a T250Q and M428L mutations. In some embodiments, the Fc region comprises L309D, Q311H, and N434S mutations. In some embodiments, the Fc region comprises L309D, Q311H, and N434Y mutations. In some embodiments, the Fc region comprises Q311R and M428L mutations. In some embodiments, the Fc region comprises Q311R, M428E, and N434W mutations. In some embodiments, the FC region comprises a P329G, LALA (L234A, L235A), LALAGA (L234A, L235A, G237A), YTE (M252Y/S254T/T256E), or LS (M428L/N434S) mutation. Mutations in the Fc region are also described in US2007041972A1, US20190048078A1, US20220348690A1, EP2235059B1, U.S. Pat. Nos. 7,365,168, 8,394,925, 11,492,415; Mueller et al. (1997) Mol. Immunol. 34(6):441-52, Schlothauer et al. (2016) Protein Eng Des Sel.; 29(10):457-466, and Damelang et al., (2024) Front. Immunol. 14:1304365 (see, e.g., Table 2), each of which is incorporated by reference in its entirety. The numbering referenced herein refers to the Kabat numbering system for the Fc region.

In embodiments, the Fc region comprises one or more of the following mutations: E233P; G236A [GA]; G237A; P238D; S239A; I253A; S254A; D265A; S267E; H268F; D270A; R292A; N297(A/Q/G) [NA]; S298N; K322A; S324T; K326W; A327Q; L328E; L328F; P329A; 1332E; E333A; K338A; E345R [Arg345]; E380A; E430G; H433A; N434A; N435W; K439E; S440K; C221D/D222C; L234A/L235A [LALA]; L234A/L235E; L234A/G237A; G236A/G237A [GAGA]; G236N/H268D; G236R/L328R [RR]; G236A/I332E [AE]; K236W/E333S [KWES]; S239D/I332E [DE or SDIE]; P247I/A339Q; M252Y/T256D [YD]; T256D/T307Q [DQ]; T256D/T307W [DW]; P257I/Q311I [PIQI]; S267E/L328F [SE/LF]; H268F/S324T [FT or HFST]; S298G/T299A [Ga]; K326A/E333A; K326M/E333S; K326W/E333S [WS]; A330S/P331S; E380A/N434A; M428L/N434S [MN or LS]; H433K/N434F [HN or KF]; E233P/L234V/L235A; L234A/L235A/K322A; L234F/L235E/K322A; L234F/L235Q/K322Q [FQQ]; L234A/L235A/P329G [LALAPG]; L234F/L235E/P331S [FES]; L234S/L235T/G236R; L234A/L235A/G237A; L234F/L235E/D265A [FEA]; L234Y/G236W/S298A [YWA]; L235A/G237A/E318A; G236A/S239D/I332E [GASDIE]; G236A/A330L/I332E [GAALIE]; S239D/S298A/I332E; S239D/A330L/I332E [SDALIE or DLE]; T250Q/M428L/N434S [QLS]; M252Y/S254T/T256E[YTE or MST]; I253A/H310A/H435A [IHH]; P257I/M428L/N434S; V259I/N315D/N434Y [C6A-74]; S267E/H268F/S324T [EFT]; H285D/T307Q/A378V [DQV]; S298A/E333A/K334A [AAA]; T307A/E380A/N434A; L309D/Q311H/N434S [DHS]; A327G/A330S/P331S; I332E/M428L/N434S; E333A/M428L/N434S [ALS]; E345R/E430G/S440Y [RGY]; D376V/M428L/N434S; E380A/M428L/N434S; L234A/L235A/N297A/P329G; L234A/L235A/M428L/N434S; G236A/S239D/A330L/1332E [GASDALIE]; S239D/H268F/S324T/I332E; S239D/I332E/M428L/N434S [SDIE LS]; P257I/Q311I/M428L/N434S [PIQI LS]; S267E/L328F/M428L/N434S [SE/LF LS]; H268F/S324T/M428L/N434S [HFST LS]; T307A/E380A/M428L/N434S; L235V/F243L/R292P/Y300L/P396L [VPLIL]; F243L/R292P/Y300L/V305I/P396L [Variant 18(LPLIL)]; G236A/S239D/I332E/M428L/N434S; G236A/S267E/H268F/S324T/I332E [EFT-EA]; S239D/S298A/I332E/M428L/N434S; S239D/A330L/I332E/M428L/N434S; M252Y/S254T/T256E/M428L/N434S [YTE LS]; M252Y/S254T/T256E/H433K/N434F [YTE-KF or MST/HN]; S267E/H268F/S324T/M428L/N434S [SEHFST]; N315D/A330V/N361D/A378V/N434Y [T5A-74]; E345R/E430G/S440Y/M428L/N434S [RGY LS]; G236A/S239D/A330L/I332E/M428L/N434S; M252Y/S254T/T256Y+S239D/A330L/I332E [YTE-SDALIE]; E233D/G237D/P238D/H268D/P271G/A330R [V12]; E233P/L234V/L235A/DG236+A327G/A330S/P331S; G236A/S267E/H268F/S324T/1332E/M428L/N434S [EFT-EA LS]; T250Q/M428L; N434Y; Q311R/M428L [PFc29]; and Q311R/M428E/N434W [REW]; or any combination thereof.

In embodiments, the Fc region comprises one or more of the following mutations: E380A; N434A; N435W; M252Y/T256D [YD]; T256D/T307Q [DQ]; T256D/T307W [DW]; M428L/N434S [MN or LS]; H433K/N434F [HN or KF]; M252Y/S254T/T256E[YTE or MST]; V259I/N315D/N434Y [C6A-74]; H285D/T307Q/A378V [DQV]; T307A/E380A/N434A; L309D/Q311H/N434S [DHS]; M252Y/S254T/T256E/H433K/N434F [YTE-KF or MST/HN]; N315D/A330V/N361D/A378V/N434Y [T5A-74]; M252Y/S254T/T256Y+S239D/A330L/I332E [YTE-SDALIE]; T250Q/M428L; N434Y; Q311R/M428L [PFc29]; and Q311R/M428E/N434W [REW]; or any combination thereof.

A “Fab′ fragment” contains one light chain and a portion or fragment of one heavy chain that contains the V_{H} domain and the C_{H}1 domain and also the region between the C_{H}1 and C _{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}² domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

The term “single-chain Fv” or “scFv” antibody refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding.

For a review of scFv, see Pluckthun (1994) The Pharmacology of Monoclonal Antibodies, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen binding sites. In some instances, the two binding sites have the same antigen specificities. However, bivalent antibodies may be bispecific (see below).

In certain embodiments, monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079). In one embodiment, the present invention provides single domain antibodies comprising two V_{H} domains with modifications such that single domain antibodies are formed.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

Typically, a variant antibody or antigen binding fragment of the antibodies provided herein retain at least 10% of its IGF-1R binding activity (when compared to a parental antibody that is modified) when that activity is expressed on a molar basis. In some embodiments, a variant antibody (or antigen fragment thereof), or antigen binding fragment of an antibody provided herein, retains at least 20%, 50%, 70%, 80%, 90%, 95% or 100% or more of the IGF-1R binding affinity as the parental antibody. As described herein, it is also intended that an antibody or antigen binding fragment of the invention can include conservative or non-conservative amino acid substitutions, which can also be referred to as “conservative variants” or “function conserved variants” of the antibody, that do not substantially alter its biologic activity.

“Isolated antibody” refers to the purification status of a binding compound and in such context means the molecule is substantially free of other biological molecules such as nucleic acids, proteins, lipids, carbohydrates, or other material such as cellular debris and growth media.

Generally, the term “isolated” is not intended to refer to a complete absence of such material or to an absence of water, buffers, or salts, unless they are present in amounts that substantially interfere with experimental or therapeutic use of the binding compound as described herein.

The term “monoclonal antibody”, as used herein, refers to population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, that are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, a “chimeric antibody” is an antibody having the variable domain from a first antibody and constant domain from a second antibody, where the first and second antibodies are from different species. (U.S. Pat. No. 4,816,567; and Morrison et al., (1984) Proc. Natl. Acad. Sci. USA 81: 6851-6855). Typically, the variable domains are obtained from an antibody from an experimental animal (the “parental antibody”), such as a rodent, and the constant domain sequences are obtained from human antibodies, so that the resulting chimeric antibody will be less likely to elicit an adverse immune response in a human subject than the parental (e.g., rodent) antibody.

As used herein, the term “humanized antibody” refers to forms of antibodies that contain sequences from both human and non-human (e.g., murine, rat) antibodies. In general, the humanized antibody will comprise substantially all of at least one, and typically two, variable domains, in which all or substantially all of the hypervariable loops correspond to those of a non-human immunoglobulin, and all or substantially all of the framework (FR) regions are those of a human immunoglobulin sequence. The humanized antibody may optionally comprise at least a portion of a human immunoglobulin constant region (Fc).

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody that comprises mouse immunoglobulin sequences only. Alternatively, a fully human antibody may contain rat carbohydrate chains if produced in a rat, in a rat cell, or in a hybridoma derived from a rat cell. Similarly, “rat antibody” refers to an antibody that comprises rat immunoglobulin sequences only.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally (unless otherwise specified), in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, the term “hypervariable region” refers to the amino acid residues of an antibody that are responsible for antigen-binding. The hypervariable region comprises amino acid residues from a “complementarity determining region” or “CDR” (i.e. residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain; Kabat et al. (1991) Sequences of Proteins of Immunological Interest, 5th Ed. Public Health Service, National Institutes of Health, Bethesda, Md.) and/or those residues from a “hypervariable loop” (i.e. residues 26-32 (CDRL1), 50-52 (CDRL2) and 91-96 (CDRL3) in the light chain variable domain and 26-32 (CDRH1), 53-55 (CDRH2) and 96-101 (CDRH3) in the heavy chain variable domain; Chothia and Lesk (1987) J. Mol. Biol. 196: 901-917). Those of ordinary skill in the art will appreciate that for a given amino acid variable region sequence, different number schemes may be used to define the CDR, such as, for example, Kabat, IMGT, Chothia, and North/Dunbrack. (North). As used herein, the term “framework” or “FR” residues refers to those variable domain residues other than the hypervariable region residues defined herein as CDR residues. CDRs provide the majority of contact residues for the binding of the antibody to the antigen or epitope. CDRs of interest can be derived from donor antibody variable heavy and light chain sequences, and include analogs of the naturally occurring CDRs, which analogs also share or retain the same antigen binding specificity and/or neutralizing ability as the donor antibody from which they were derived.

Additionally, in some embodiments, the antibodies can take the form of a full length antibody, single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; a Microbody; a peptide aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; Affimers, a DuoBody, a Fv, a Fab, a Fab′, a F(ab′)2, a peptide mimetic molecule, or a synthetic molecule, as described in US Patent Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of each of which are hereby incorporated by reference in their entireties. See also, Storz (2011) MAbs 3(3): 310-317, which is hereby incorporated by reference. In some embodiments, the antibodies can take the form of a bispecific, a trispecific antibody, a multispecific antibody, a diabody, a triabody, a tetrabody, a minibody, a sterol regulatory binding protein cleavage activating protein (Scap), a chelating recombinant antibody, an intrabody, or a small modular immunopharmaceutical (SMIP), which can collectively be referred to as forms of an antibody.

The term “antigen” as used herein means any molecule that has the ability to generate antibodies either directly or indirectly or that binds to antibody. Included within the definition of “antigen” is a protein-encoding nucleic acid. An “antigen” can also refer to the binding partner of an antibody. In some embodiments, the antigen is the IGF-1R protein expressed on the surface of a cell. In some embodiments, the cell is an intact cell. An intact cell is a cell that has not been lysed or broken open with the use of detergents or other reagents. A cell that has been treated with detergents or other reagents that breaks up the cellular membrane or punches holes in a cellular membrane is not an intact cell. For example, methods are provided herein for generating an antibody that binds to an IGF-1R protein, the method comprising culturing a cell comprising a nucleic acid molecule encoding the IGF-1R antibody.

As used herein, “specific binding” or “immunospecific binding” or “binds immunospecifically” refer to antibody binding to a predetermined antigen (e.g., IGF-1R) or epitope present on the antigen. In some embodiments, the antibody binds with a dissociation constant (K_{D}) of 10⁻⁷ M or less, and binds to the predetermined antigen with a K_{D} that is at least two-fold less than its K_{D} for binding to a non-specific antigen (e.g., BSA, casein, or another non-specific polypeptide) other than the predetermined antigen. The phrases “an antibody recognizing IGF-1R “an antibody specific for IGF-1R” are used interchangeably herein with the term “an antibody which binds immunospecifically to IGF-1R.” Reference in the present disclosure may be made to IGF-1R. The degree of specificity necessary for an anti-IGF-1R antibody may depend on the intended use of the antibody, and at any rate is defined by its suitability for use for an intended purpose. In some embodiments, the antibody, or binding compound derived from the antigen-binding site of an antibody, of the contemplated method binds to its antigen (IGF-1R), with an affinity that is at least two fold greater, at least ten times greater, at least 20-times greater, or at least 100-times greater than the affinity with any other antigen.

Methods for determining mAb specificity and affinity by competitive inhibition can be found in Harlow, et al., Antibodies: A Laboratory Manual, Cold Spring Harbor Laboratory Press, Cold Spring Harbor, N.Y., (1988), Colligan et al., eds., Current Protocols in Immunology, Greene Publishing Assoc. and Wiley Interscience, N.Y., (1992, 1993), and Muller (1983) Meth. Enzymol. 92:589-601, which references are entirely incorporated herein by reference.

The term “homolog” means protein sequences having between 40% and 100% sequence homology or identity to a reference sequence. Percent identity between two peptide chains can be determined by pair wise alignment using the default settings of the AlignX module of Vector NTI v.9.0.0 (Invitrogen Corp., Carslbad, Calif). In some embodiments, the antibody, or antigenic binding fragment thereof has, at least 50, 60, 70, 80, 90, 91, 92, 93, 94, 95, 96, 97, 98, or 99% homology or identity to a sequence described herein. In some embodiments, the antibody has conservative substitutions as compared to a sequence described herein. Exemplary conservative substitutions are illustrated in Table 1 and are encompassed within the scope of the disclosed subject matter. The conservative substitution may reside in the framework regions, or in antigen-binding sites, as long they do not adversely affect the properties of the antibody. Substitutions may be made to improve antibody properties, for example stability or affinity. Conservative substitutions will produce molecules having functional and chemical characteristics similar to those molecules into which such modifications are made. Exemplary amino acid substitutions are shown in the table below.

TABLE 1
Exemplary Conservative Substitutions:

| Original Residue | Exemplary Conservative Substitutions |
|---|---|
| Ala | Val, Leu, Ile |
| Arg | Lys, Gln, Asn |
| Asn | Gln |
| Asp | Glu |
| Cys | Ser, Ala |
| Gln | Asn |
| Gly | Pro, Ala |
| His | Asn, Gln, Lys, Arg |
| Ile | Leu, Val, Met, Ala, Phe |
| Leu | Ile, Val, Met, Ala, Phe |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

In some embodiments, variants of the proteins and peptides provided herein are provided. In some embodiments, a variant comprises a substitution, deletions, or insertion. In some embodiments, the variant comprises 1, 2, 3, 4, 5, 6, 7, 8, 9, or 10 (e.g., 1-10) substitutions. As described herein, the substitutions can be conservative substitutions. In some embodiments, the substitution is non-conservative. In some embodiments, the variant comprises 1, 2, 3, 4, 5, 6, 7, 8, 9, or 10 (e.g., 1-10) deletions. In some embodiments, the variant comprises 1, 2, 3, 4, 5, 6, 7, 8, 9, or 10 (e.g., 1-10) insertions. In some embodiments, the substitutions, deletions, or insertions are present in the CDRs provided for herein. In some embodiments, the substitutions, deletions, or insertions are not present in the CDRs provided for herein.

The term “in combination with” as used herein means that the described agents can be administered to an animal or subject together in a mixture, concurrently as single agents or sequentially as single agents in any order.

The techniques to raise antibodies to small peptide sequences that recognize and bind to those sequences in the free or conjugated form or when presented as a native sequence in the context of a large protein are well known in the art. Such antibodies include murine, murine-human and human-human antibodies produced by hybridoma or recombinant techniques known in the art. Antibodies can also be produced in human, a mouse, sheep, a rat, a rabbit, a shark, a llama, or a chicken. In some embodiments, the antibody is produced in a chicken. The antibodies can also be produced in or other small animals.

The term “epitope” is meant to refer to that portion of any molecule capable of being recognized by and bound by an antibody at one or more of the Ab's antigen binding regions. Epitopes usually consist of chemically active surface groupings of molecules such as amino acids or sugar side chains and have specific three-dimensional structural characteristics as well as specific charge characteristics. Example of epitopes include, but are not limited to, the residues described herein that form IGF-1R epitopes. In some embodiments, the epitope is only present in a non-denatured protein. In some embodiments, the epitope is only present in a denatured protein.

In some embodiments, the source for the DNA encoding a non-human antibody include cell lines which produce antibody, such as hybrid cell lines commonly known as hybridomas.

The hybrid cells are formed by the fusion of a non-human antibody-producing cell, typically a spleen cell of an animal immunized against either natural or recombinant antigen, or a peptide fragment of the antigen protein sequence. Alternatively, the non-human antibody-producing cell can be a B lymphocyte obtained from the blood, spleen, lymph nodes or other tissue of an animal immunized with the antigen.

The second fusion partner, which provides the immortalizing function, can be a lymphoblastoid cell or a plasmacytoma or myeloma cell, which is not itself an antibody producing cell, but is malignant. Fusion partner cells include, but are not limited to, the hybridoma SP2/0-Ag14, abbreviated as SP2/0 (ATCC CRL1581) and the myeloma P3X63Ag8 (ATCC TIB9), or its derivatives. See, e.g., Ausubel infra, Harlow infra, and Colligan infra, the contents of which references are incorporated entirely herein by reference.

The antibodies can be generated according to the examples provided herein. Once the sequences are known, the antibodies can also be generated according to known methods. The antibodies can also be converted to different types, such as being converted to Human IgGs and the like. By converting the antibodies to a human antibody, a human subject should not identify the antibodies as foreign. The conversion of a non-human IgG antibody to a human IgG antibody is well known and can routinely be done once the native sequence is known. As discussed herein, the antibodies can be modified according to known methods. Such methods are described in, for example, Riechmann L et al. (1988) Reshaping human antibodies for therapy Nature 332(6162): 332-323; Tsurushita N et al. (2004) J. Immunol. Methods 295(1-2): 9-19. The antibody-producing cell contributing the nucleotide sequences encoding the antigen-binding region of the chimeric antibody can also be produced by transformation of a non-human, such as a primate, or a human cell. For example, a B lymphocyte which produces the antibody can be infected and transformed with a virus such as Epstein-Barr virus to yield an immortal antibody producing cell (Kozbor et al., (1983) Immunol. Today 4:72 79). Alternatively, the B lymphocyte can be transformed by providing a transforming gene or transforming gene product, as is well-known in the art. See, e.g., Ausubel infra, Harlow infra, and Colligan infra, the contents of which references are incorporated entirely herein by reference. The cell fusions are accomplished by standard procedures well known to those skilled in the field of immunology. Fusion partner cell lines and methods for fusing and selecting hybridomas and screening for mAbs are well known in the art. See, e.g., Ausubel infra, Harlow infra, and Colligan infra, the contents of which references are incorporated entirely herein by reference.

In some embodiments, the antibody is a MAb or antibody fragment, or a form, variant, or derivative thereof, which binds to IGF-1R. In some embodiments, the antibody (e.g., MAb or antibody fragment) binds to amino acids of an epitope of the IGF-1R.

In some embodiments, the antibody comprises a sequence as provided for herein.

The sequences of the antibodies can be modified to yield human IgG antibodies. The conversion of the sequences provided herein can be modified to yield other types of antibodies. The CDRs can also be linked to other antibodies, proteins, or molecules to create antibody fragments that bind to IGF-1R. This can be in the form of an antibody drug conjugate (“ADC”), a multi-specific molecule, or a chimeric antigen receptor. The CDRs and antibody sequences provided herein also be humanized or made fully human according to known methods. The sequences can also be made into chimeric antibodies as described herein.

In some embodiments, the antibody comprises an amino acid sequence comprising a sequence provided for herein or a fragment thereof. In some embodiments, the antibody comprises one or more amino acid sequences as provided herein, an antigen binding fragments, thereof, or a human IgG variant thereof. “A human IgG variant thereof” refers to an antibody that has been modified to be a human IgG when the starting antibody is not a human IgG antibody.

As described herein the production of antibodies with a known sequence is routine and can be done by any method. Accordingly, in some embodiments, a nucleic acid encoding an antibody or fragment thereof is provided. In some embodiments, the nucleic acid encodes a sequence provided for herein. The antibodies can also be modified to be chimeric antibodies or human antibodies. The antibodies can also be used in injectable pharmaceutical compositions. As also described herein, the antibodies can be isolated antibodies or engineered antibodies.

In some embodiments, methods described herein comprise use or administration of an anti-IGF-1R antibody, and antigen binding fragments thereof, including any forms, variants, or derivatives thereof of the anti-IGF-1R antibody, and antigen binding fragments.

In some embodiments, “derivatives” of the antibodies, fragments, regions or derivatives thereof, which term includes those proteins encoded by truncated or modified genes to yield molecular species functionally resembling the immunoglobulin fragments are provided. The modifications include, but are not limited to, addition of genetic sequences coding for cytotoxic proteins such as plant and bacterial toxins. The modification can also include a reporter protein, such as a fluorescent or chemiluminescent tag. The fragments and derivatives can be produced in any manner.

In some embodiments, the antibody is a derivative thereof that binds to IGF-1R. In some embodiments, the antibody (e.g., antibody derivative) binds to amino acids of an epitope of the IGF-1R.

In some embodiments, the antibody is an antibody variant that binds to IGF-1R. In some embodiments, the antibody variant binds to amino acids of an epitope of the IGF-1R.

In some embodiments, the antibody is any form of an antibody as described herein. In some embodiments, the antibody form binds to amino acids of an epitope of the IGF-1R.

The identification of these antigen binding region and/or epitopes recognized by Abs described herein provide the information necessary to generate additional monoclonal antibodies with similar binding characteristics and therapeutic or diagnostic utility that parallel the embodiments of this application.

The nucleic acid sequence encoding an antibody described herein can be genomic DNA or cDNA, or RNA (e.g., mRNA) which encodes at least one of the variable regions described herein. A convenient alternative to the use of chromosomal gene fragments as the source of DNA encoding the V region antigen-binding segment is the use of cDNA for the construction of chimeric immunoglobulin genes, e.g., as reported by Liu et al. (1987) Proc. Natl. Acad. Sci. 84:3439 and Liu et al. (1987) J. Immunology 139:3521, which references are hereby entirely incorporated herein by reference. The use of cDNA requires that gene expression elements appropriate for the host cell be combined with the gene in order to achieve synthesis of the desired protein. The use of cDNA sequences is advantageous over genomic sequences (which contain introns), in that cDNA sequences can be expressed in bacteria or other hosts which lack appropriate RNA splicing systems.

For example, a cDNA encoding a V region antigen-binding segment able to detect, bind, to or neutralize an IGF-1R antigen can be provided using known methods based on the use of the amino acid sequences provided herein. Because the genetic code is degenerate, more than one codon can be used to encode a particular amino acid (Watson, et al., infra). Using the genetic code, one or more different oligonucleotides can be identified, each of which would be capable of encoding the amino acid. The probability that a particular oligonucleotide will, in fact, constitute the actual XXX-encoding sequence can be estimated by considering abnormal base pairing relationships and the frequency with which a particular codon is actually used (to encode a particular amino acid) in eukaryotic or prokaryotic cells expressing an antibody or fragment. Such “codon usage rules” are disclosed by Lathe et al. (1985) J. Molec. Biol. 183:1-12. Using the “codon usage rules” of Lathe, a single oligonucleotide, or a set of oligonucleotides, that contains a theoretical “most probable” nucleotide sequence capable of encoding an antibody variable or constant region sequences is identified.

The variable regions described herein can be combined with any type of constant region including a human constant region or murine constant region. Human genes which encode the constant (C) regions of the antibodies, fragments and regions can be derived from a human fetal liver library, by known methods. Human C regions genes can be derived from any human cell including those which express and produce human immunoglobulins. The human C_{H} region can be derived from any of the known classes or isotypes of human H chains, including gamma, p, a, 6 or 8, and subtypes thereof, such as G1, G2, G3 and G4. Since the H chain isotype is responsible for the various effector functions of an antibody, the choice of C_{H} region will be guided by the desired effector functions, such as complement fixation, or activity in antibody-dependent cellular cytotoxicity (ADCC). Preferably, the C_{H} region is derived from gamma 1 (IgG1), gamma 3 (IgG3), gamma 4 (IgG4), or μ (IgM). The human C_{L} region can be derived from either human L chain isotype, kappa or lambda. In some embodiments, the antibody comprises a Fc domain. In some embodiments, the Fc domain comprises a mutation to extend the half-life of the antibody. In some embodiments, the Fc domain comprises a mutation such as those described in U.S. Pat. No. 7,670,600, which is hereby incorporated by reference in its entirety. In some embodiment, the constant region comprises a mutation at position at amino acid residue 428 relative to a wild-type human IgG constant domain, numbered according to the EU numbering index of Kabat. Without being bound to any particular theory, an antibody comprising a mutation that corresponds to residue 428 can have an increased half-life compared to the half-life of an IgG having the wild-type human IgG constant domain. In some embodiments, the mutation is a substitution of the native residue with a threonine, leucine, phenylalanine or serine. In some embodiments, the antibody further comprises one or more amino acid substitutions relative to the corresponding wild-type human IgG constant domain at one or more of amino acid residues 251-256, 285-290, 308-314, 385-389, and 429-436, numbered according to the Kabat EU numbering index. The specific mutations or substitutions at these positions are described in U.S. Pat. No. 7,670,600, which is hereby incorporated by reference in its entirety.

Genes encoding human immunoglobulin C regions can be obtained from human cells by standard cloning techniques (Sambrook et al. (Molecular Cloning: A Laboratory Manual, 2nd Edition, Cold Spring Harbor Press, Cold Spring Harbor, N.Y. (1989) and Ausubel et al., eds. Current Protocols in Molecular Biology (1987)). Human C region genes are readily available from known clones containing genes representing the two classes of L chains, the five classes of H chains and subclasses thereof. Chimeric antibody fragments, such as F(ab′)₂ and Fab, can be prepared by designing a chimeric H chain gene which is appropriately truncated. For example, a chimeric gene encoding an H chain portion of an F(ab′)₂ fragment would include DNA sequences encoding the C_{H}1 domain and hinge region of the H chain, followed by a translational stop codon to yield the truncated molecule.

In some embodiments, the antibodies, murine, human, humanized, or chimeric antibodies, fragments and regions of the antibodies described herein are produced by cloning DNA segments encoding the H and L chain antigen-binding regions of an IGF-1R antigen specific antibody, and joining these DNA segments to DNA segments encoding C_{H} and C_{L} regions, respectively, to produce murine, human or chimeric immunoglobulin-encoding genes.

Thus, in some embodiments, a fused chimeric gene is created which comprises a first DNA segment that encodes at least the antigen-binding region of non-human origin, such as a functionally rearranged V region with joining (J) segment, linked to a second DNA segment encoding at least a part of a human C region.

Therefore, cDNA encoding the antibody V and C regions, the method of producing the antibody according to some of the embodiments described herein can involve several steps, for example: 1. isolation of messenger RNA (mRNA) from the cell line producing an anti-IGF-1R antigen antibody and from optional additional antibodies supplying heavy and light constant regions; cloning and cDNA production therefrom; 2. preparation of a full length cDNA library from purified mRNA from which the appropriate V and/or C region gene segments of the L and H chain genes can be: (i) identified with appropriate probes, (ii) sequenced, and (iii) made compatible with a C or V gene segment from another antibody for a chimeric antibody; 3. construction of complete H or L chain coding sequences by linkage of the cloned specific V region gene segments to cloned C region gene, as described above; and 4. expression and production of L and H chains in selected hosts, including prokaryotic and eukaryotic cells to provide murine-murine, human-murine, human-human or human murine antibodies.

Two coding DNA sequences are said to be “operably linked” if the linkage results in a continuously translatable sequence without alteration or interruption of the triplet reading frame. A DNA coding sequence is operably linked to a gene expression element if the linkage results in the proper function of that gene expression element to result in expression of the coding sequence.

As used herein and unless otherwise indicated, the term “about” is intended to mean±5% of the value it modifies. Thus, about 100 means 95 to 105.

In some embodiments, the antibodies described herein are used to detect the presence of the antigen. The present antibody can be used in any device or method to detect the presence of the antigen.

The term “purified” with reference to an antibody refers to an antibody that is substantially free of other material that associates with the molecule in its natural environment.

For instance, a purified protein is substantially free of the cellular material or other proteins from the cell or tissue from which it is derived. The term refers to preparations where the isolated protein is sufficiently pure to be analyzed, e.g., at least 70% to 80% (w/w) pure, at least 80%-90% (w/w) pure, 90-95% pure; or at least 95%, 96%, 97%, 98%, 99%, or 100% (w/w) pure. In some embodiments, the antibody is purified.

As an alternative to preparing monoclonal antibody-secreting hybridomas, a monoclonal antibody to a polypeptide may be identified and isolated by screening a recombinant combinatorial immunoglobulin library (e.g., an antibody phage display library) with a polypeptide described herein to thereby isolate immunoglobulin library members that bind to the polypeptide.

Techniques and commercially available kits for generating and screening phage display libraries are well known to those skilled in the art. Additionally, examples of methods and reagents particularly amenable for use in generating and screening antibody or antigen binding protein display libraries can be found in the literature. Thus, the epitopes described herein can be used to screen for other antibodies that can be used therapeutically, diagnostically, or as research tools.

### Antibody Conjugates

The antibodies provided for herein may also be conjugated to a chemical moiety. The chemical moiety may be, inter alia, a polymer, a radionuclide or a cytotoxic factor.

In some embodiments, this can be referred to as an antibody drug conjugate. In some embodiments, the chemical moiety is a polymer which increases the half-life of the antibody molecule in the body of a subject. Suitable polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee et al. (1999) Bioconj. Chem. 10:973-981 discloses PEG conjugated single-chain antibodies. Wen et al. (2001) Bioconj. Chem. 12:545-553 disclose conjugating antibodies with PEG which is attached to a radiometal chelator (diethylenetriaminpentaacetic acid (DTPA)). Examples of chemical moieties include, but are not limited to, anti-mitotics, such as calicheamicins (e.g., ozogamicin), monomethyl auristatin E, mertansine, and the like. Other examples include, but are not limited to, biologically active anti-microtubule agents, alkylating agents and DNA minor groove binding agents. Other examples of are provided herein and below. The chemical moiety can be linked to the antibody through a linking group (maleimide), a cleavable linker, such as a cathepsin cleavable linkers (valine-citrulline), and in some embodiments, one or more spacers (e.g., para-aminobenzylcarbamate). Without being bound to any particular theory, once the antibody conjugate binds IGF-1R it can be internalized and the chemical moiety can kill the cell or otherwise inhibit its growth. In some embodiments, the cell is a thyroid cell.

The antibodies and antibody fragments of the invention may also be conjugated with labels such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr and ⁵⁶Fe.

The antibodies and antibody fragments may also be conjugated with fluorescent or chemiluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The antibody molecules may also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, Momordica charantia inhibitor, curcin, crotin, Saponaria officinalis inhibitor, mitogellin, restrictocin, phenomycin, and enomycin.

Any method known in the art for conjugating the antibody molecules of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies are conventional and very well known in the art.

### Chimeric Antigen Receptors

The antibodies provided herein can also be incorporated into a chimeric antigen receptor (“CAR”) that can be used, for example, in a CAR-T cell. In some embodiments, the extracellular domain of the CAR can be an antibody as provided for herein. In some embodiments, the antibody is in a scFv format. CAR-T cells are a type of treatment in which a patient's T cells are modified so they will attack the cells that are expressing IGF-1R. T cells are taken from a patient's blood. Then the gene for a special receptor that binds to a certain protein on the patient's cells is added in the laboratory. In some embodiments, the receptor binds to IGF-1R using the binding regions of the antibodies provided for herein. The CAR-T cells comprising the IGF-1R antibody can then be used to treat a condition, such as those provided for herein.

### Anti-IGF-1R Antibodies

In some embodiments, antibodies (e.g., an anti-IGF-1R antibody) are provided herein. In some embodiments, the antibody is a recombinant antibody that binds to an IGF-1R protein. In some embodiments, the IGF-1R protein is a human IGF-1R protein. In some embodiments, the IGF-1R protein that is recognized by the antibodies is in its native conformation (non-denatured) conformation. In some embodiments, the antibody does not specifically bind to a denatured IGF-1R protein. As used herein, the term “recombinant antibody” refers to an antibody that is not naturally occurring. In some embodiments, the term “recombinant antibody” refers to an antibody that is not isolated from a human subject.

In some embodiments, the antibody comprises one or more peptides having the following sequences, or a variant thereof:

TABLE 2
Anti-IGF-1R Antibodies

| AB ID NQ. | AB Sequence | VL Sequence | VH Sequence |
|---|---|---|---|
| VRDN-5000 | DVVMTQTPLSLPVSLGDPASISC | DVVMTQTPLSLPVSL | QVQLVQSGAEVVKPGASVKLSC |
| | RSSQSIVHSNVNTYLEWYLQKPG | GDPASISCRSSQSIV | KASGYTFTSYWMHWVKQRPGQG |
| | QSPRLLIYKVSNRFSGVPDRESG | HSNVNTYLEWYLQKP | LEWIGEINPSNGRTNYNQKFQG |
| | SGAGTDFTLRISRVEAEDLGIYY | GQSPRLLIYKVSNRF | KATLTVDKSSSTAYMQLSSLTS |
| | CFQGSHVPPTFGGGTKLEIKRTV | SGVPDRFSGSGAGTD | EDSAVYYFARGRPDYYGSSKWY |
| | AAPSVFIFPPSDEQLKSGTASVV | FTLRISRVEAEDLGI | FDVWGQGTTVTVSS (SEQ ID |
| | CLLNNFYPREAKVQWKVDNALQS | YYCFQGSHVPPTFGG | NO: 3) |
| | GNSQESVTEQDSKDSTYSLSSTL | GTKLEIKR (SEQ | |
| | TLSKADYEKHKVYACEVTHQGLS | ID NO: 2) | |
| | SPVTKSENRGEC (SEQ ID | | |
| | NO: 11) | | |
| | QVQLVQSGAEVVKPGASVKLSCK | | |
| | ASGYTFTSYWMHWVKQRPGQGLE | | |
| | WIGEINPSNGRTNYNQKFQGKAT | | |
| | LTVDKSSSTAYMQLSSLTSEDSA | | |
| | VYYFARGRPDYYGSSKWYFDVWG | | |
| | QGTTVTVSSASTKGPSVFPLAPS | | |
| | SKSTSGGTAALGCLVKDYFPEPV | | |
| | TVSWNSGALTSGVHTFPAVLQSS | | |
| | GLYSLSSVVTVPSSSLGTQTYIC | | |
| | NVNHKPSNTKVDKKVEPKSCDKT | | |
| | HTCPPCPAPELLGGPSVFLFPPK | | |
| | PKDTLMISRTPEVTCVVVDVSHE | | |
| | DPEVKFNWYVDGVEVHNAKTKPR | | |
| | EEQYNSTYRVVSVLTVLHQDWLN | | |
| | GKEYKCKVSNKALPAPIEKTISK | | |
| | AKGQPREPQVYTLPPSRDELTKN | | |
| | QVSLTCLVKGFYPSDIAVEWESN | | |
| | GQPENNYKTTPPVLDSDGSFFLY | | |
| | SKLTVDKSRWQQGNVFSCSVMHE | | |
| | ALHNHYTQKSLSLSPGK (SEQ | | |
| | ID NO: 10) | | |
| VRDN-003 | DVVMTQTPLSLPVSLGDPASISC | DVVMTQTPLSLPVSL | QVQLVQSGAEVVKPGASVKLSC |
| | RSSQSIVHSNVNTYLEWYLQKPG | GDPASISCRSSQSIV | KASGYTFTSYWMHWVKQRPGQG |
| | QSPRLLIYKVSNRFSGVPDRESG | HSNVNTYLEWYLQKP | LEWIGEINPSNGRTNYNQKFQG |
| | SGAGTDFTLRISRVEAEDLGIYY | GQSPRLLIYKVSNRF | KATLTVDKSSSTAYMQLSSLTS |
| | CFQGSHVPPTFGGGTKLEIKRTV | SGVPDRFSGSGAGTD | EDSAVYYFARGRPDYYGSSKWY |
| | AAPSVFIFPPSDEQLKSGTASVV | FTLRISRVEAEDLGI | FDVWGQGTTVTVSS (SEQ ID |
| | CLLNNFYPREAKVQWKVDNALQS | YYCFQGSHVPPTFGG | NO: 3) |
| | GNSQESVTEQDSKDSTYSLSSTL | GTKLEIK (SEQ ID | |
| | TLSKADYEKHKVYACEVTHQGLS | NO: 2) | |
| | SPVTKSENRGEC (SEQ ID | | |
| | No: 15) | | |
| VRDN-002 | QVQLVQSGAEVVKPGASVKLSCK | | |
| | ASGYTFTSYWMHWVKQRPGQGLE | | |
| | WIGEINPSNGRTNYNQKFQGKAT | | |
| | LTVDKSSSTAYMQLSSLTSEDSA | | |
| | VYYFARGRPDYYGSSKWYFDVWG | | |
| | QGTTVTVSSASTKGPSVFPLAPS | | |
| | SKSTSGGTAALGCLVKDYFPEPV | | |
| | TVSWNSGALTSGVHTFPAVLQSS | | |
| | GLYSLSSVVTVPSSSLGTQTYIC | | |
| | NVNHKPSNTKVDKKVEPKSCDKT | | |
| | HTCPPCPAPELLGGPSVFLFPPK | | |
| | PKDTLYITREPEVTCVVVDVSHE | | |
| | DPEVKFNWYVDGVEVHNAKTKPR | | |
| | EEQYNSTYRVVSVLTVLHQDWLN | | |
| | GKEYKCKVSNKALPAPIEKTISK | | |
| | AKGQPREPQVYTLPPSRDELTKN | | |
| | QVSLTCLVKGFYPSDIAVEWESN | | |
| | GQPENNYKTTPPVLDSDGSFFLY | | |
| | SKLTVDKSRWQQGNVFSCSVMHE | | |
| | ALHNHYTQKSLSLSPG (SEQ | | |
| | ID NO: 14) | | |
| | DIVMTQSPLSLPVTPGEPASISC | DIVMTQSPLSLPVTP | QVQLQESGPGLVKPSETLSLTC |
| | RSSQSIVHSNGNTYLQWYLQKPG | GEPASISCRSSQSIV | TVSGYSITGGYLWNWIRQPPGK |
| | QSPQLLIYKVSNRLYGVPDRFSG | HSNGNTYLQWYLQKP | GLEWIGYISYDGTNNYKPSLKD |
| | SGSGTDFTLKISRVEAEDVGVYY | GQSPQLLIYKVSNRL | RVTISRDTSKNQFSLKLSSVTA |
| | CFQGSHVPWTFGQGTKVEIKRTV | YGVPDRFSGSGSGTD | ADTAVYYCARYGRVFFDYWGQG |
| | AAPSVFIFPPSDEQLKSGTASVV | FTLKISRVEAEDVGV | TLVTVSS (SEQ ID NO: |
| | CLLNNFYPREAKVQWKVDNALQS | YYCFQGSHVPWTFGQ | 17) |
| | GNSQESVTEQDSKDSTYSLSSTL | GTKVEIKR (SEQ | |
| | TLSKADYEKHKVYACEVTHQGLS | ID NO: 16) | |
| | SPVTKSENRGEC (SEQ ID | | |
| | NO: 25) | | |
| | QVQLQESGPGLVKPSETLSLTCT | | |
| | VSGYSITGGYLWNWIRQPPGKGL | | |
| | EWIGYISYDGTNNYKPSLKDRVT | | |
| | ISRDTSKNQFSLKLSSVTAADTA | | |
| | VYYCARYGRVFFDYWGQGTLVTV | | |
| | SSASTKGPSVFPLAPSSKSTSGG | | |
| | TAALGCLVKDYFPEPVTVSWNSG | | |
| | ALTSGVHTFPAVLQSSGLYSLSS | | |
| | VVTVPSSSLGTQTYICNVNHKPS | | |
| | NTKVDKRVEPKSCDKTHTCPPCP | | |
| | APELLGGPSVFLFPPKPKDTLYI | | |
| | TREPEVTCVVVDVSHEDPEVKEN | | |
| | WYVDGVEVHNAKTKPREEQYNST | | |
| | YRVVSVLTVLHQDWLNGKEYKCK | | |
| | VSNKALPAPIEKTISKAKGQPRE | | |
| | PQVYTLPPSREEMTKNQVSLTCL | | |
| | VKGFYPSDIAVEWESNGQPENNY | | |
| | KTTPPVLDSDGSFFLYSKLTVDK | | |
| | SRWQQGNVFSCSVMHEALHNHYT | | |
| | QKSLSLSPG (SEQ ID NO: | | |
| | 24) | | |

The V_{H} and the V_{L} sequences can be in any format, including, but not limited to a scFv format where the V_{H} and V_{L} regions are linked with a peptide linker. Examples of peptide linkers that can be used to link various peptides provided for herein include, but are not limited to: (GGGGS)ₙ (SEQ ID NO: 12); (GGGGA)ₙ (SEQ ID NO: 13), or any combination thereof, wherein each n is independently 1-5. In some embodiments, the peptide linker comprises (GGGGS)ₙ (SEQ ID NO: 12), (GGGGA)ₙ (SEQ ID NO: 13), (GSTSGSGKPGSGEGSTKG)ₙ (SEQ ID NO: 26) or any combination thereof, wherein each n is independently 1-8. In some embodiments, the variable regions are not linked with a peptide linker. In some embodiments, the antibody comprises or consists of a polypeptide set forth in SEQ ID NOS: 10 and 11. In some embodiments, the antibody comprises a polypeptide comprising SEQ ID NOS: 3, 4, 5, 6, 7, 8, and 9. In some embodiments, the antibody comprises a polypeptide comprising SEQ ID NOS: 27, 28, 6, 29, 30, and 31. In some embodiments, the antibody comprises a polypeptide comprising SEQ ID NOS: 4, 5, 6, 32, 33, and 9. In some embodiments, the antibody comprises a polypeptide comprising SEQ ID NOS: 4, 34, 6, 35, 36, and 31. In some embodiments, the antibody comprises a polypeptide comprising SEQ ID NOS: 18, 19, 20, 21, 22, and 23. In some embodiments, the antibody comprises a polypeptide comprising SEQ ID NOS: 37, 28, 20, 38, 39, and 40. In some embodiments, the antibody comprises a polypeptide comprising SEQ ID NOS: 18, 19, 20, 41, 42, and 23. In some embodiments, the antibody comprises a polypeptide comprising SEQ ID NOS: 18, 43, 20, 44, 45, and 40.

In some embodiments, an antibody, or antigen binding fragment thereof is provided, wherein the antibody or antibody fragment comprises a peptide selected from the following tables.

TABLE 3
Kabat CDRs

| Ab ID No | LCDR1 | LCDR2 | LCDR3 | HCDR1 | HCDR2 | HCDR3 |
|---|---|---|---|---|---|---|
| VRDN-5000 | RSSQSIVH | KVSNRFS | FQGSHVPPT | SYWMH | GEINPSNGRTNYNQ | GRPDYYGSSK |
| /VRDN-003 | SNVNTYLE | (SEQ ID | (SEQ ID | (SEQ ID | KFQG (SEQ ID | WYFDV (SEQ |
| | (SEQ ID | NO: 5) | NO: 6) | NO: 7) | NO: 8) | ID NO: 9) |
| | NO: 4) | | | | | |
| VRDN-002 | RSSQSIVH | KVSNRLY | FQGSHVPWT | GGYLWN | YISYDGTNNYKPSL | YGRVFFDY |
| | SNGNTYLQ | (SEQ ID | (SEQ ID | (SEQ ID | KD (SEQ ID NO: | (SEQ ID |
| | WYLQ | NO: 19) | NO: 20) | NO: 21) | 22) | NO: 23) |
| | (SEQ ID | | | | | |
| | NO: 18) | | | | | |

TABLE 4
IMGT CDRs

| Ab ID No | LCDR1 | LCDR2 | LCDR3 | HCDR1 | HCDR2 | HCDR3 |
|---|---|---|---|---|---|---|
| VRDN-5000 | QSIVHSNV | KVS (SEQ | FQGSHVPPT | GYTFTSYW | INPSNGRT | ARGRPDYYGS |
| /VRDN-003 | NTY (SEQ | ID NO: | (SEQ ID | (SEQ ID | (SEQ ID | SKWYFDV |
| | ID NO: | 28) | NO: 6) | NO: 29) | NO: 30) | (SEQ ID |
| | 27) | | | | | NO: 31) |
| VRDN-002 | QSIVHSNG | KVS (SEQ | FQGSHVPWT | GYSITGGY | ISYDGTN | ARYGRVFFDY |
| | NTY (SEQ | ID NO: | (SEQ ID | L (SEQ | (SEQ ID | (SEQ ID |
| | ID 37) | 28) | NO: 20) | ID NO: | NO: 39) | NO: 40) |
| | | | | 38) | | |

TABLE 5
Chothia CDRs

| Ab ID No | LCDR1 | LCDR2 | LCDR3 | HCDR1 | HCDR2 | HCDR3 |
|---|---|---|---|---|---|---|
| VRDN-5000 | RSSQSIVH | KVSNRFS | FQGSHVPPT | GYTFTSY | NPSNGR | GRPDYYGSSK |
| /VRDN-003 | SNVNTYLE | (SEQ ID | (SEQ ID | (SEQ ID | (SEQ ID | WYFDV (SEQ |
| | (SEQ ID | NO: 5) | NO: 6) | NO: 32) | NO: 33) | ID NO: 9) |
| | NO: 4) | | | | | |
| VRDN-002 | RSSQSIVH | KVSNRLY | FQGSHVPWT | GYSITGGY | SYDGT | YGRVFFDY |
| | SNGNTYLQ | (SEQ ID | (SEQ ID | (SEQ ID | (SEQ ID | (SEQ ID |
| | WYLQ | NO: 19) | NO: 20) | NO: 41) | NO: 42) | NO: 23) |
| | (SEQ ID | | | | | |
| | NO: 18) | | | | | |

TABLE 6
North CDRs

| Ab ID No | LCDR1 | LCDR2 | LCDR3 | HCDR1 | HCDR2 | HCDR3 |
|---|---|---|---|---|---|---|
| VRDN-5000 | RSSQSIVH | YKVSNRFS | FQGSHVPPT | KASGYTFT | EINPSNGRTN | ARGRPDYYGS |
| /VRDN-003 | SNVNTYLE | (SEQ ID | (SEQ ID | SYWMH | (SEQ ID NO: | SKWYFDV |
| | (SEQ ID | NO: 34) | NO: 6) | (SEQ ID | 36) | (SEQ ID |
| | NO: 4) | | | NO: 35) | | NO: 31) |
| VRDN-002 | RSSQSIVH | YKVSNRLY | FQGSHVPWT | TVSGYSIT | YISYDGTNN (SEQ | ARYGRVFFDY |
| | SNGNTYLQ | (SEQ ID | (SEQ ID | GGYLWN | ID NO: 45) | (SEQ ID |
| | WYLQ | NO: 43) | NO: 20) | (SEQ ID | | NO: 40) |
| | (SEQ ID | | | NO: 44) | | |
| | NO: 18) | | | | | |

In some embodiments, the antibody comprises one or more peptides having the following sequences, or a variant thereof comprising one or more variable domain (italicized) CDRs (italics and bold, according to Kabat numbering scheme) and a human IgG1/Kappa constant domain (underlined):

TABLE 7
Anti-IGF-1R Antibodies

| | |
|---|---|
| VRDN-5000_HC (SEQ | QVQLVQSGAEVVKPGASVKLSCKASGYTFTSYWMHWVKQRPGQGLEWIGEINPSNGRT |
| ID NO: 10) | NYNQKFQGKATLTVDKSSSTAYMQLSSLTSEDSAVYYFARGRPDYYGSSKWYFDVWGQ |
| | GTTVTVSSASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKKVEPKSCDKTHT |
| | CPPCPAPELLGGPSVFLFPPKPKDTLMISRTPEVTCVVVDVSHEDPEVKENWYVDGVE |
| | VHNAKTKPREEQYNSTYRVVSVLTVLHQDWLNGKEYKCKVSNKALPAPIEKTISKAKG |
| | QPREPQVYTLPPSRDELTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| VRDN-5000_LC (SEQ | DVVMTQTPLSLPVSLGDPASISCRSSQSIVHSNVNTYLEWYLQKPGQSPRLLIYKVSN |
| ID NO: 11) | RFSGVPDRESGSGAGTDFTLRISRVEAEDLGIYYCFQGSHVPPTFGGGTKLEIKRTVA |
| | APSVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSENRGEC |
| VRDN-003_HC (SEQ ID | QVQLVQSGAEVVKPGASVKLSCKASGYTFTSYWMHWVKQRPGQGLEWIGEINPSNGRT |
| NO: 14) | NYNQKFQGKATLTVDKSSSTAYMQLSSLTSEDSAVYYFARGRPDYYGSSKWYFDVWGQ |
| | GTTVTVSSASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKKVEPKSCDKTHT |
| | CPPCPAPELLGGPSVFLFPPKPKDTLYITREPEVTCVVVDVSHEDPEVKENWYVDGVE |
| | VHNAKTKPREEQYNSTYRVVSVLTVLHQDWLNGKEYKCKVSNKALPAPIEKTISKAKG |
| | QPREPQVYTLPPSRDELTKNQVSLTCLVKGFYPSDIAVEWESNGQPENNYKTTPPVLD |
| | SDGSFFLYSKLTVDKSRWQQGNVFSCSVMHEALHNHYTQKSLSLSPG |
| VRDN-003_LC (SEQ ID | DVVMTQTPLSLPVSLGDPASISCRSSQSIVHSNVNTYLEWYLQKPGQSPRLLIYKVSN |
| NO: 15) | RFSGVPDRESGSGAGTDFTLRISRVEAEDLGIYYCFQGSHVPPTFGGGTKLEIKRTVA |
| | APSVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSENRGEC |
| VRDN-002_HC (SEQ ID | QVQLQESGPGLVKPSETLSLTCTVSGYSITGGYLWNWIRQPPGKGLEWIGYISYDGTN |
| NO: 24) | NYKPSLKDRVTISRDTSKNQFSLKLSSVTAADTAVYYCARYGRVFFDYWGQGTLVTVS |
| | SASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKRVEPKSCDKTHTCPPCPAP |
| | ELLGGPSVFLFPPKPKDTLYITREPEVTCVVVDVSHEDPEVKENWYVDGVEVHNAKTK |
| | PREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFL |
| | YSKLTVDKSRWQQGNVFSCSVMHEALHNHYTQKSLSLSPG |
| VRDN-002_LC (SEQ ID | DIVMTQSPLSLPVTPGEPASISCRSSQSIVHSNGNTYLQWYLQKPGQSPQLLIYKVSN |
| NO: 25) | RLYGVPDRFSGSGSGTDFTLKISRVEAEDVGVYYCFQGSHVPWTFGQGTKVEIKRTVA |
| | APSVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSENRGEC |

In some embodiments, an antibody, or antibody binding fragment thereof, comprises a heavy or light chain having a sequence of SEQ ID NOs: 10 and 11. In some embodiments, an antibody, or an antibody binding fragment thereof, comprises a heavy chain having a sequence of SEQ ID NO: 10. In some embodiments, an antibody, or an antibody binding fragment thereof, comprises a heavy chain having a sequence that is 80%, 81%, 82%, 83% 84%, 85%, 86%, 87%, 88%, 89%, 90%, 91%, 92%, 93%, 94%, 95%, 96%, 97%, 98%, 99%, 99.5% 99.9% or substantially 100% identical to that of SEQ ID NO: 10. In some embodiments, an antibody, or an antibody binding fragment thereof, comprises a heavy chain having a sequence that is 95%, 96%, 97%, 98%, 99%, 99.5%, 99.9% or substantially 100% identical to that of SEQ ID NO: 10. In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 10 comprises the CDRs of SEQ ID NO: 7, 8, and/or 9 as set forth above. In some embodiments, the sequence that is 95%, 96%, 97%, 98%, 99%, 99.5%, 99.9% or substantially 100% identical to that of SEQ ID NO: 10 comprises the CDRs of SEQ ID NO: 7, 8, and/or 9 as set forth above. In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 10 comprises the CDRs of SEQ ID NO: 29, 30, and/or 31 as set forth above. In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 10 comprises the CDRs of SEQ ID NO: 32, 33, and/or 9 as set forth above. In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 10 comprises the CDRs of SEQ ID NO: 35, 36, and/or 31 as set forth above.

In some embodiments, an antibody, or an antibody binding fragment thereof, comprises a light chain having a sequence of SEQ ID NO: 11. In some embodiments, an antibody, or an antibody binding fragment thereof, comprises a heavy chain having a sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 11.

In some embodiments, an antibody, or an antibody binding fragment thereof, comprises a light chain having a sequence that is 95%, 96%, 97%, 98%, 99%, 99.5%, 99.9% or substantially 100% identical to that of SEQ ID NO: 11 In some embodiments, the sequence that is 95%, 96%, 97%, 98%, 99%, 99.5%, 99.9% or substantially 100% identical to that of SEQ ID NO: 11 comprises the CDRs of SEQ ID NO: 4, 5, and/or 6 as set forth above. In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 11 comprises the CDRs of SEQ ID NO: 4, 5, and/or 6 as set forth above. In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 11 comprises the CDRs of SEQ ID NO: 27, 28, and/or 6 as set forth above. In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 11 comprises the CDRs of SEQ ID NO: 4, 34 and/or 6 as set forth above.

In some embodiments, an antibody, or antibody binding fragment thereof, comprises a light chain CDR having a sequence of SEQ ID NO: 4, 5, or 6. In some embodiments, an antibody, or antibody binding fragment thereof comprises a heavy chain CDR having a sequence of SEQ ID NO: 7, 8, or 9.

In some embodiments, an antibody, or antibody binding fragment thereof, comprises a light chain having a LCDR1, a LCDR2, and a LCDR3, wherein the LCDR1 has a sequence of SEQ ID NO: 4 the LCDR2 has a sequence of SEQ ID NO: 5 and the LCDR3 has a sequence of SEQ ID NO: 6.

In some embodiments, an antibody, or antibody binding fragment thereof, comprises a heavy chain having a HCDR1, a HCDR2, and a HCDR3, wherein the HCDR1 has a sequence of SEQ ID NO: 7 the HCDR2 has a sequence of SEQ ID NO: 8 and the HCDR3 has a sequence of SEQ ID NO: 9.

In some embodiments, an antibody, or antibody binding fragment thereof, comprises a light chain CDR having a sequence of SEQ ID NO: 27, 28, or 6. In some embodiments, an antibody, or antibody binding fragment thereof comprises a heavy chain CDR having a sequence of SEQ ID NO: 29, 30, or 31.

In some embodiments, an antibody, or antibody binding fragment thereof, comprises a light chain having a LCDR1, a LCDR2, and a LCDR3, wherein the LCDR1 has a sequence of SEQ ID NO: 27 the LCDR2 has a sequence of SEQ ID NO: 28 and the LCDR3 has a sequence of SEQ ID NO: 6.

In some embodiments, an antibody, or antibody binding fragment thereof, comprises a heavy chain having a HCDR1, a HCDR2, and a HCDR3, wherein the HCDR1 has a sequence of SEQ ID NO: 29 the HCDR2 has a sequence of SEQ ID NO: 30 and the HCDR3 has a sequence of SEQ ID NO: 31.

In some embodiments, an antibody, or antibody binding fragment thereof comprises a heavy chain CDR having a sequence of SEQ ID NO: 32, 33, or 9.

In some embodiments, an antibody, or antibody binding fragment thereof, comprises a heavy chain having a HCDR1, a HCDR2, and a HCDR3, wherein the HCDR1 has a sequence of SEQ ID NO: 32 the HCDR2 has a sequence of SEQ ID NO: 33 and the HCDR3 has a sequence of SEQ ID NO: 9.

In some embodiments, an antibody, or antibody binding fragment thereof, comprises a light chain CDR having a sequence of SEQ ID NO: 4, 34, or 6. In some embodiments, an antibody, or antibody binding fragment thereof comprises a heavy chain CDR having a sequence of SEQ ID NO: 35, 36, or 31.

In some embodiments, an antibody, or antibody binding fragment thereof, comprises a light chain having a LCDR1, a LCDR2, and a LCDR3, wherein the LCDR1 has a sequence of SEQ ID NO: 4 the LCDR2 has a sequence of SEQ ID NO: 34 and the LCDR3 has a sequence of SEQ ID NO: 6.

In some embodiments, an antibody, or antibody binding fragment thereof, comprises a heavy chain having a HCDR1, a HCDR2, and a HCDR3, wherein the HCDR1 has a sequence of SEQ ID NO: 35 the HCDR2 has a sequence of SEQ ID NO: 36 and the HCDR3 has a sequence of SEQ ID NO: 31.

The different CDR motifs can be combined in any combination including those not depicted in the table above. For example, the following embodiments are provided as non-limiting examples of such combinations.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 4; the light chain CDR2 has the amino acid sequence of SEQ ID NO: 5; and the light chain CDR3 sequence has the amino acid sequence of SEQ ID NO: 6; and (ii) a heavy chain variable region comprising heavy chain CDR1, CDR2, and CDR3 sequences, wherein the heavy chain CDR1 sequence has the amino acid sequence of SEQ ID NO: 7; the heavy chain CDR2 sequence has the amino acid sequence of SEQ ID NO: 8; and the heavy chain CDR3 sequence has the amino acid sequence of SEQ ID NO: 9; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 27; the light chain CDR2 has the amino acid sequence of SEQ ID NO: 28; and the light chain CDR3 sequence has the amino acid sequence of SEQ ID NO: 6; and (ii) a heavy chain variable region comprising heavy chain CDR1, CDR2, and CDR3 sequences, wherein the heavy chain CDR1 sequence has the amino acid sequence of SEQ ID NO: 29; the heavy chain CDR2 sequence has the amino acid sequence of SEQ ID NO: 30; and the heavy chain CDR3 sequence has the amino acid sequence of SEQ ID NO: 31; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 4; the light chain CDR2 has the amino acid sequence of SEQ ID NO: 5; and the light chain CDR3 sequence has the amino acid sequence of SEQ ID NO: 6; and (ii) a heavy chain variable region comprising heavy chain CDR1, CDR2, and CDR3 sequences, wherein the heavy chain CDR1 sequence has the amino acid sequence of SEQ ID NO: 32; the heavy chain CDR2 sequence has the amino acid sequence of SEQ ID NO: 33; and the heavy chain CDR3 sequence has the amino acid sequence of SEQ ID NO: 9; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 4; the light chain CDR2 has the amino acid sequence of SEQ ID NO: 34; and the light chain CDR3 sequence has the amino acid sequence of SEQ ID NO: 6; and (ii) a heavy chain variable region comprising heavy chain CDR1, CDR2, and CDR3 sequences, wherein the heavy chain CDR1 sequence has the amino acid sequence of SEQ ID NO: 35; the heavy chain CDR2 sequence has the amino acid sequence of SEQ ID NO: 36; and the heavy chain CDR3 sequence has the amino acid sequence of SEQ ID NO: 31; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence that is 95%, 96%, 97%, 98%, 99%, 99.5%, 99.9% or substantially 100% identical to that of SEQ ID NO: 4; the light chain CDR2 has the amino acid sequence that is 95%, 96%, 97%, 98%, 99%, 99.5%, 99.9% or substantially 100% identical to that of SEQ ID NO: 5; and the light chain CDR3 sequence has the amino acid sequence that is 95%, 96%, 97%, 98%, 99%, 99.5%, 99.9% or substantially 100% identical to that of SEQ ID NO: 6; and (ii) a heavy chain variable region comprising heavy chain CDR1, CDR2, and CDR3 sequences, wherein the heavy chain CDR1 sequence has the amino acid sequence that is 95%, 96%, 97%, 98%, 99%, 99.5%, 99.9% or substantially 100% identical to that of SEQ ID NO: 7; the heavy chain CDR2 sequence has the amino acid sequence that is 95%, 96%, 97%, 98%, 99%, 99.5%, 99.9% or substantially 100% identical to that of SEQ ID NO: 8; and the heavy chain CDR3 sequence has the amino acid sequence that is 95%, 96%, 97%, 98%, 99%, 99.5%, 99.9% or substantially 100% identical to that of SEQ ID NO: 9; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 4; the light chain CDR2 has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 5; and the light chain CDR3 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 6; and (ii) a heavy chain variable region comprising heavy chain CDR1, CDR2, and CDR3 sequences, wherein the heavy chain CDR1 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 7; the heavy chain CDR2 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 8; and the heavy chain CDR3 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 9; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 27; the light chain CDR2 has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 28; and the light chain CDR3 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 6; and (ii) a heavy chain variable region comprising heavy chain CDR1, CDR2, and CDR3 sequences, wherein the heavy chain CDR1 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 29; the heavy chain CDR2 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 30; and the heavy chain CDR3 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 31; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 4; the light chain CDR2 has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 5; and the light chain CDR3 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 6; and (ii) a heavy chain variable region comprising heavy chain CDR1, CDR2, and CDR3 sequences, wherein the heavy chain CDR1 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 32; the heavy chain CDR2 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 33; and the heavy chain CDR3 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 9; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 4; the light chain CDR2 has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 34; and the light chain CDR3 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 6; and (ii) a heavy chain variable region comprising heavy chain CDR1, CDR2, and CDR3 sequences, wherein the heavy chain CDR1 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 35; the heavy chain CDR2 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 36; and the heavy chain CDR3 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 31; or variants of any of the foregoing.

In some embodiments, an antibody, or antibody binding fragment thereof, comprises a heavy or light chain having a sequence of SEQ ID NOs: 14 and 15. In some embodiments, an antibody, or an antibody binding fragment thereof, comprises a heavy chain having a sequence of SEQ ID NO: 14. In some embodiments, an antibody, or an antibody binding fragment thereof, comprises a heavy chain having a sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 14. In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 14 comprises the CDRs of SEQ ID NO: 7, 8, and/or 9 as set forth above. In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 14 comprises the CDRs of SEQ ID NO: 29, 30, and/or 31 as set forth above.

In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 14 comprises the CDRs of SEQ ID NO: 32, 33, and/or 9 as set forth above. In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 14 comprises the CDRs of SEQ ID NO: 35, 36, and/or 31 as set forth above.

In some embodiments, an antibody, or an antibody binding fragment thereof, comprises a light chain having a sequence of SEQ ID NO: 15. In some embodiments, an antibody, or an antibody binding fragment thereof, comprises a light chain having a sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 15.

In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 15 comprises the CDRs of SEQ ID NO: 4, 5, and/or 6 as set forth above.

In some embodiments, the antibody, or antigen binding fragment thereof, or protein is provided that comprises a peptide having a sequence as set forth in any of SEQ ID NOs: 1, 2, 3, 4, 5, 6, 7, 8, 9, 10, 11, 14, 15, 27, 28, 29, 30, 31, 32, 33, 34, 35, or 36.

In some embodiments, an antibody, or antibody binding fragment thereof, comprises a heavy or light chain having a sequence of SEQ ID NOs: 24 and 25. In some embodiments, an antibody, or an antibody binding fragment thereof, comprises a heavy chain having a sequence of SEQ ID NO: 24. In some embodiments, an antibody, or an antibody binding fragment thereof, comprises a heavy chain having a sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 24. In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 24 comprises the CDRs of SEQ ID NO: 21, 22, and/or 23 as set forth above. In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 24 comprises the CDRs of SEQ ID NO: 38, 39, and/or 40 as set forth above.

In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 24 comprises the CDRs of SEQ ID NO: 41, 42, and/or 23 as set forth above. In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 24 comprises the CDRs of SEQ ID NO: 44, 45, and/or 40 as set forth above.

In some embodiments, an antibody, or an antibody binding fragment thereof, comprises a light chain having a sequence of SEQ ID NO: 25. In some embodiments, an antibody, or an antibody binding fragment thereof, comprises a light chain having a sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 25.

In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 25 comprises the CDRs of SEQ ID NO: 18, 19, and/or 20 as set forth above. In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 25 comprises the CDRs of SEQ ID NO: 37, 28, and/or 20 as set forth above. In some embodiments, the sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 25 comprises the CDRs of SEQ ID NO: 18, 43, and/or 20 as set forth above.

In some embodiments, an antibody, or antibody binding fragment thereof, comprises a light chain CDR having a sequence of SEQ ID NO: 18, 19, or 20. In some embodiments, an antibody, or antibody binding fragment thereof comprises a heavy chain CDR having a sequence of SEQ ID NO: 21, 22, or 23.

In some embodiments, an antibody, or antibody binding fragment thereof, comprises a light chain having a LCDR1, a LCDR2, and a LCDR3, wherein the LCDR1 has a sequence of SEQ ID NO: 18 the LCDR2 has a sequence of SEQ ID NO: 19 and the LCDR3 has a sequence of SEQ ID NO: 20.

In some embodiments, an antibody, or antibody binding fragment thereof, comprises a heavy chain having a HCDR1, a HCDR2, and a HCDR3, wherein the HCDR1 has a sequence of SEQ ID NO: 21 the HCDR2 has a sequence of SEQ ID NO: 22 and the HCDR3 has a sequence of SEQ ID NO: 23.

In some embodiments, an antibody, or antibody binding fragment thereof, comprises a light chain CDR having a sequence of SEQ ID NO: 18, 19, or 20. In some embodiments, an antibody, or antibody binding fragment thereof comprises a heavy chain CDR having a sequence of SEQ ID NO: 21, 22, or 23.

In some embodiments, an antibody, or antibody binding fragment thereof, comprises a light chain having a LCDR1, a LCDR2, and a LCDR3, wherein the LCDR1 has a sequence of SEQ ID NO: 37 the LCDR2 has a sequence of SEQ ID NO: 28 and the LCDR3 has a sequence of SEQ ID NO: 20.

In some embodiments, an antibody, or antibody binding fragment thereof, comprises a heavy chain having a HCDR1, a HCDR2, and a HCDR3, wherein the HCDR1 has a sequence of SEQ ID NO: 38 the HCDR2 has a sequence of SEQ ID NO: 39 and the HCDR3 has a sequence of SEQ ID NO: 40.

In some embodiments, an antibody, or antibody binding fragment thereof comprises a heavy chain CDR having a sequence of SEQ ID NO: 41, 42, or 23.

In some embodiments, an antibody, or antibody binding fragment thereof, comprises a heavy chain having a HCDR1, a HCDR2, and a HCDR3, wherein the HCDR1 has a sequence of SEQ ID NO: 41 the HCDR2 has a sequence of SEQ ID NO: 42 and the HCDR3 has a sequence of SEQ ID NO: 23.

In some embodiments, an antibody, or antibody binding fragment thereof, comprises a light chain CDR having a sequence of SEQ ID NO: 18, 43, or 20. In some embodiments, an antibody, or antibody binding fragment thereof comprises a heavy chain CDR having a sequence of SEQ ID NO: 44, 45, or 40.

In some embodiments, an antibody, or antibody binding fragment thereof, comprises a light chain having a LCDR1, a LCDR2, and a LCDR3, wherein the LCDR1 has a sequence of SEQ ID NO: 18 the LCDR2 has a sequence of SEQ ID NO: 43 and the LCDR3 has a sequence of SEQ ID NO: 20.

In some embodiments, an antibody, or antibody binding fragment thereof, comprises a heavy chain having a HCDR1, a HCDR2, and a HCDR3, wherein the HCDR1 has a sequence of SEQ ID NO: 44 the HCDR2 has a sequence of SEQ ID NO: 45 and the HCDR3 has a sequence of SEQ ID NO: 40.

The different CDR motifs can be combined in any combination including those not depicted in the table above. For example, the following embodiments are provided as non-limiting examples of such combinations.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 18; the light chain CDR2 has the amino acid sequence of SEQ ID NO: 19; and the light chain CDR3 sequence has the amino acid sequence of SEQ ID NO: 20; and (ii) a heavy chain variable region comprising heavy chain CDR1, CDR2, and CDR3 sequences, wherein the heavy chain CDR1 sequence has the amino acid sequence of SEQ ID NO: 21; the heavy chain CDR2 sequence has the amino acid sequence of SEQ ID NO:22; and the heavy chain CDR3 sequence has the amino acid sequence of SEQ ID NO: 23; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 37; the light chain CDR2 has the amino acid sequence of SEQ ID NO: 28; and the light chain CDR3 sequence has the amino acid sequence of SEQ ID NO: 20; and (ii) a heavy chain variable region comprising heavy chain CDR1, CDR2, and CDR3 sequences, wherein the heavy chain CDR1 sequence has the amino acid sequence of SEQ ID NO: 38; the heavy chain CDR2 sequence has the amino acid sequence of SEQ ID NO:39; and the heavy chain CDR3 sequence has the amino acid sequence of SEQ ID NO: 40; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 18; the light chain CDR2 has the amino acid sequence of SEQ ID NO: 19; and the light chain CDR3 sequence has the amino acid sequence of SEQ ID NO: 20; and (ii) a heavy chain variable region comprising heavy chain CDR1, CDR2, and CDR3 sequences, wherein the heavy chain CDR1 sequence has the amino acid sequence of SEQ ID NO: 41; the heavy chain CDR2 sequence has the amino acid sequence of SEQ ID NO:42; and the heavy chain CDR3 sequence has the amino acid sequence of SEQ ID NO: 23; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 18; the light chain CDR2 has the amino acid sequence of SEQ ID NO: 43; and the light chain CDR3 sequence has the amino acid sequence of SEQ ID NO: 20; and (ii) a heavy chain variable region comprising heavy chain CDR1, CDR2, and CDR3 sequences, wherein the heavy chain CDR1 sequence has the amino acid sequence of SEQ ID NO: 44; the heavy chain CDR2 sequence has the amino acid sequence of SEQ ID NO:45; and the heavy chain CDR3 sequence has the amino acid sequence of SEQ ID NO: 40; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 18; the light chain CDR2 has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 19; and the light chain CDR3 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 20; and (ii) a heavy chain variable region comprising heavy chain CDR1, CDR2, and CDR3 sequences, wherein the heavy chain CDR1 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 21; the heavy chain CDR2 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 22; and the heavy chain CDR3 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 23; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 37; the light chain CDR2 has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 28; and the light chain CDR3 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 20; and (ii) a heavy chain variable region comprising heavy chain CDR1, CDR2, and CDR3 sequences, wherein the heavy chain CDR1 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 38; the heavy chain CDR2 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 39; and the heavy chain CDR3 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 40; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 37; the light chain CDR2 has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 28; and the light chain CDR3 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 20; and (ii) a heavy chain variable region comprising heavy chain CDR1, CDR2, and CDR3 sequences, wherein the heavy chain CDR1 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 41; the heavy chain CDR2 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 42; and the heavy chain CDR3 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 23; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 18; the light chain CDR2 has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 43; and the light chain CDR3 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 20; and (ii) a heavy chain variable region comprising heavy chain CDR1, CDR2, and CDR3 sequences, wherein the heavy chain CDR1 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 44; the heavy chain CDR2 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 45; and the heavy chain CDR3 sequence has the amino acid sequence that is 80%, 81%, 82%, 83% 84%, 85%, 86%, 87%, 88%, 89%, 90%, 91%, 92%, 93%, 94%, 95%, 96%, 97%, 98%, 99%, 99.5%, 99.9% or substantially 100% identical to that of SEQ ID NO: 40; or variants of any of the foregoing.

In some embodiments, the antibody, or antigen binding fragment thereof, or protein is provided that comprises a peptide having a sequence as set forth in any of SEQ ID NOs: 16, 17, 18, 19, 20, 21, 22, 23, 24, 25, 28, 37, 38, 39, 40, 41, 42, 43, 44, or 45.

In some embodiments, methods described herein comprise use or administration of an anti-IGF-1R antibody, and antigen binding fragments thereof, including any forms, variants, or derivatives thereof of the anti-IGF-1R antibody, and antigen binding fragments.

In some embodiments, the antibody, or antigen binding fragment thereof, comprises a sequence of, or a variant of any of the foregoing.

In some embodiments, an anti-IGF-1R antibody comprises an anti-IGF-1R antibody as discloses in WO 2023/122714 (hereby incorporated by reference in its entirety), or a variant thereof.

In some embodiments, an anti-IGF-1R antibody comprises an anti-IGF-1R antibody as discloses in WO 2023/133485 (hereby incorporated by reference in its entirety), or a variant thereof.

In some embodiments, an anti-IGF-1R antibody comprises an anti-IGF-1R antibody as discloses in WO 2023/133486 (hereby incorporated by reference in its entirety), or a variant thereof.

### Pharmaceutical Compositions

In some embodiments, to prepare pharmaceutical or sterile compositions of the anti-IGF-1R antibodies or other proteins provided herein, the antibody or antigen binding fragment thereof or other proteins provided herein are admixed with a pharmaceutically acceptable carrier or excipient. See, e.g., Remington's Pharmaceutical Sciences and U.S. Pharmacopeia: National Formulary, Mack Publishing Company, Easton, PA (1984).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY). In some embodiments, the antibodies are diluted to an appropriate concentration in a sodium acetate solution pH 5-6, and NaCl or sucrose is added for tonicity. Additional agents, such as polysorbate 20 or polysorbate 80, may be added to enhance stability.

Toxicity and therapeutic efficacy of the antibody compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, antibodies exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

In some embodiments, a composition of the invention is administered to a subject in accordance with the Physicians' Desk Reference 2003 (Thomson Healthcare; 57th edition (Nov. 1, 2002)).

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In some embodiments, the antibody or antigen binding fragment thereof can be administered by an invasive route such as by injection. In some embodiments, the antibodies or antigen binding fragment thereof, or pharmaceutical composition thereof, is administered intravenously, subcutaneously, intramuscularly, intraarterially, intra-articularly (e.g., in arthritis joints), or by inhalation, aerosol delivery. Administration by non-invasive routes (e.g., orally; for example, in a pill, capsule or tablet) is also within the scope of the present embodiments.

In some embodiments, the anti-IGF-1R antibody, or antigen binding fragment thereof, is administered in combination with at least one additional therapeutic agent, such as, but not limited to any therapeutic used to treat thyroid eye disease. For example, in some embodiments, the anti-IGF-1R antibody, or antigen binding fragment thereof, is administered in combination with at least one additional therapeutic agent, such as, but not limited to a therapeutic used to treat thyroid eye disease or a condition related to the same. Examples of such treatments and therapeutics include, but are not limited to anti-thyroid medications, diabetes medications, beta-blockers, immunosuppressants, propylthiouracil, methimazole, propranolol, atenolol, metoprolol, nadolol, corticosteroids, metformin, sulfonylureas, meglitinides, thiazolidinediones, DPP-4 inhibitors, GLP-1 receptor agonists, SGLT2 inhibitors, regular insulin, insulin aspart, insulin glulisine, insulin lispro, insulin isophane, insulin degludec, insulin detemir, insulin glargine, acerbose, miglitol, acebutolol, atenolol, betaxolol, bisoprolol, cartelol, carvedilol, esmolol, labetalol, metoprolol, nadolol, nebivolol, penbutolol, pindolol, propranolol, sotalol, timolol, tomolol ophthalmic solution, sitagliptin, saxagliptin, linagliptin, alogliptin, dulaglutide, exenatide, semaglutide, liraglutide, lixisenatide, canagliflozin, dapagliflozin, empagliflozin, or any combination thereof.

Compositions can be administered with medical devices known in the art. For example, a pharmaceutical composition of the invention can be administered by injection with a hypodermic needle, including, e.g., a prefilled syringe or autoinjector.

The pharmaceutical compositions may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

The pharmaceutical compositions may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternately, one may administer the antibody in a local rather than systemic manner, for example, via injection of the antibody directly into an arthritic joint or pathogen-induced lesion characterized by immunopathology, often in a depot or sustained release formulation. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

### IGF-1R Associated Pathologies and Thyroid Eye Disease

As used herein, an IGF-1R associated pathology refers to conditions that are caused by the modulation of IGF-1R. These conditions include, but are not limited to, thyroid eye disease and other conditions provided for herein.

In some embodiments, methods of treating, inhibiting or ameliorating an IGF-1R, associated pathology are provided. In some embodiments, the methods comprise administering an antibody described herein or a pharmaceutical composition described herein to a subject to treat, inhibit or ameliorate an IGF-1R associated pathology. In some embodiments, the pathology is as described herein. In some embodiments, an IGF-1R associated pathology comprises thyroid-associated ophthalmopathy (TAO), also known as thyroid eye disease (TED), Graves' ophthalmopathy or orbitopathy (GO), thyrotoxic exophthalmos, dysthyroid ophthalmopathy, autoimmune associated eye disorders associated with IGF-1R signaling, or inflammatory orbital disorder associated with IGF-1R signaling. In some embodiments, an IGF-1R associated pathology comprises thyroid eye disease (TED). In some embodiments, the pathology is as described herein. In some embodiments, an IGF-1R associated pathology is fibrosis.

For example, methods described herein can be beneficial for treating thyroid eye disease in a patient (e.g., a patient with chronic thyroid disease, a patient having one or more symptoms of thyroid eye disease for at least 12 months, a patient having inactive thyroid disease, or a patient having a Clinical Activity Score of 2 or less prior to treatment). Other exemplary methods described herein include treatment of fibrosis.

### Thyroid Eye Disease

In some embodiments, an antibody for use in the treatment of thyroid associated ophthalmopathy in a subject in need thereof, is provided. In some embodiments, an anti-IGF-1R antibody for use in the treatment of thyroid associated ophthalmopathy in a subject in need thereof, is provided.

In some embodiments, the patient has moderate to severe, thyroid eye disease. The severity of the disease can be measured in the following non-limiting embodiments.

For example, for lid aperture, the distance between the lid margins are measured (in mm) with the patient looking in the primary position, sitting relaxed, and with distant fixation. Lid retraction may be assessed by measuring the lid aperture along the midline of the pupil with the patient looking in the primary position while sitting relaxed and with distant fixation (mid-pupil to upper lid margin distance). For swelling of the eyelids, the measure/evaluation is either “absent/equivocal,” “moderate,” or “severe.” Redness of the eyelids is either absent or present.

Redness of the conjunctivae is either absent or present. In some embodiments, conjunctival edema is either absent or present. In some embodiments, inflammation of the caruncle or plica is either absent or present. In some embodiments, spontaneous retrobulbar pain is either absent or present.

In some embodiments, pain on attempted eye movements (e.g., upward, side-to-side, and downward gazes) is either absent or present. Exophthalmos is measured in millimeter using the same Hertel exophthalmometer and same intercanthal distance for an individual patient. Subjective diplopia is scored from 0 to 3 (0=no diplopia; 1=intermittent, i.e., diplopia in primary position of gaze, when tired or when first awakening; 2=inconstant, i.e., diplopia at extremes of gaze; 3=constant, i.e., continuous diplopia in primary or reading position). For eye muscle involvement, the ductions are measured in degrees. Corneal involvement is either absent/punctate or keratopathy/ulcer. For optic nerve involvement, i.e., best-corrected visual acuity, color vision, optic disc, relative afferent pupillary defect, the condition is either absent or present. In addition, visual fields are checked if optic nerve compression is suspected. In some embodiments, the patient can be classified according to the following severity classification. For example, sight-Threatening Thyroid Eye Disease: Patients with dysthyroid optic neuropathy (DON) and/or corneal breakdown. This category warrants immediate intervention. Moderate-to-Severe Thyroid Eye Disease: Patients without sight-threatening disease whose eye disease have sufficient impact on daily life to justify the risks of immunosuppression (if active) or surgical intervention (if inactive). Patients with moderate-to-severe thyroid eye disease usually have any one or more of the following: lid retraction greater than or equal to 2 mm, moderate or severe soft tissue involvement, exophthalmos greater than or equal to 3 mm above normal for race and gender, inconstant or constant diplopia. Mild Thyroid Eye Disease: Patients whose features of thyroid eye disease have only a minor impact on daily life insufficient to justify immunosuppressive or surgical treatment. They usually have only one or more of the following: minor lid retraction (<2 mm), mild soft tissue involvement, exophthalmos <3 mm above normal for race and gender, transient or no diplopia, and corneal exposure responsive to lubricants.

### Active and Inactive Thyroid Eye Disease

Thyroid eye disease can be described as “active” thyroid eye disease or “inactive” thyroid disease. In some embodiments, “active” and “inactive” thyroid disease are based on differing values of the Clinical Activity Score (CAS).

As used herein, the term Clinical Activity Score (CAS) refers to the protocol described and scored according to Table 8. According to this protocol, one point is given for the presence of each of the parameters assessed in the Table below. The sum of all points defines clinical activity and provides the CAS, where 0 or 1 constitutes inactive disease and 7 severe active ophthalmopathy.

TABLE 8
Parameters for calculating Clinical Activity Score

| Item No. | Parameter |
|---|---|
| 1 | Spontaneous retrobulbar pain |
| 2 | Pain on attempted eye movements (upward, side to side, and |
| | downward gazes; sometimes termed gaze evoked orbital pain |
| 3 | Eyelid swelling |
| 4 | Eyelid erythema (redness) |
| 5 | Conjunctival redness |
| 6 | Chemosis (swelling/edema of the conjunctiva) |
| 7 | Swelling of caruncle or pila |

As provided in Table 8, the CAS consists of seven components: spontaneous retrobulbar pain, pain on attempted eye movements (upward, side-to-side, and downward gazes), conjunctival redness, redness of the eyelids, chemosis, swelling of the caruncle/plica, and swelling of the eyelids. Each component is scored as present (1 point) or absent (0 points). The score at each efficacy assessment is the sum of all items present; giving a range of 0-7, where 0 or 1 constitutes inactive disease and 7 severe active ophthalmopathy. A change of ≥2 points is considered clinically meaningful. In some embodiments, the subject's score improves by at least 2, 3, or 4 points. In some embodiments, the subject's score improves within 3 weeks of the first dose. In some embodiments, the subject's score improves within 6 weeks of the first dose.

Item 1, spontaneous orbital pain could be a painful, or oppressive feeling on, or behind, the globe. This pain may be caused by the rise in intraorbital pressure, when the orbital tissues volume increases through excess synthesis of extracellular matrix, fluid accumulation, and cellular infiltration and expansion. Item 2, gaze evoked orbital pain, could be pain in the eyes when looking, or attempting to look, up, down or sideways, i.e., pain with upward, downward, or lateral eye movement, or when attempting eye movement. This kind of pain could arise from the stretching of the inflamed muscle(s), especially on attempted upgaze. The ‘stretching pain’ cannot be provoked by digital pressing on the eyeball, as would be expected if it were a manifestation of the raised intraorbital pressure. Both kinds of pain can be reduced after anti-inflammatory treatment. These kinds of pain are therefore considered to be directly related to autoimmune inflammation in the orbit and thus useful in assessing TED activity.

Swelling in TED is seen as chemosis (edema of the conjunctiva), item no. 6 in Table 8, and swelling of the caruncle and/or plica semilunaris. Both are signs of TED activity. Swollen eyelids can be caused by edema, fat prolapse through the orbital septum, or fibrotic degeneration. In addition to swelling, other symptoms indicative of active TED include redness and/or pain of the conjunctiva, eyelid, caruncle and/or plica semilunaris.

In embodiments, a patient has a Clinical Activity Score (CAS) from about 0 to about 7. In some embodiments, the patient has a CAS greater than or equal to 2. In some embodiments, the patient has a CAS greater than or equal to 3. In some embodiments, the patient has a CAS greater than or equal to 4. In some embodiments, the patient has a CAS greater than or equal to 5. In some embodiments, the patient has a CAS greater than or equal to 6. In some embodiments, the patient has a CAS equal to 7. In embodiments, a patient, prior to treatment, had a CAS of or greater than 0, 1, 2, 3, or 4. In embodiments, a patient, prior to treatment, had a CAS less than 2. In embodiments, a patient, prior to treatment, had a CAS of 2-4. In embodiments, a patient, prior to treatment, has had a Clinical Activity Score (CAS) of greater than 3.

In some embodiments, the subject, prior to administration of the pharmaceutical composition, had a CAS of greater than 0, 1, 2, 3, or 4, or about 2 to about 4. In some embodiments, the subject, prior to administration of the pharmaceutical composition, had a CAS of greater than or equal to 2, 3, or 4. In some embodiments, the subject, prior to administration of the pharmaceutical composition, had a CAS of greater than or equal to 2. In some embodiments, the subject, prior to administration of the pharmaceutical composition, had a CAS of greater than or equal to 3. In some embodiments, the subject, prior to administration of the pharmaceutical composition, had a CAS of greater than or equal to 4.

In some embodiments, a patient has exophthalmos (proptosis) that is greater than or equal to 3 mm and a CAS from about 0 to about 7.

In some embodiments, a patient suffering from thyroid eye disease (TED) has inactive thyroid eye disease (TED). In some embodiments, a patient suffering from inactive thyroid eye disease (TED), prior to treatment, had a Clinical Activity Score (CAS) of 2 or less. In some embodiments, a patient suffering from inactive thyroid eye disease (TED), prior to treatment, had a Clinical Activity Score (CAS) of 0 or 1.

In some embodiments, a patient suffering from thyroid eye disease (TED) had active thyroid eye disease (TED). In some embodiments, a patient suffering from thyroid eye disease (TED) had a Clinical Activity Score (CAS) of greater than 0, 1, 2, 3, or 4, or about 2 to about 4. In some embodiments, a patient suffering from active thyroid eye disease (TED), prior to treatment, had a Clinical Activity Score (CAS) of greater than 2. In some embodiments, a patient suffering from active thyroid eye disease (TED), prior to treatment, had a Clinical Activity Score (CAS) of greater than 3. In some embodiments, a patient suffering from active thyroid eye disease (TED), prior to treatment, had a Clinical Activity Score (CAS) of greater than 4. In some embodiments, a patient suffering from active thyroid eye disease (TED), prior to treatment, had a Clinical Activity Score (CAS) of greater than 5. In some embodiments, a patient suffering from active thyroid eye disease (TED), prior to treatment, had a Clinical Activity Score (CAS) of greater than 6. In some embodiments, a patient suffering from active thyroid eye disease (TED), prior to treatment, had a Clinical Activity Score (CAS) of about 2 to about 4.

In some embodiments, a patient suffering from thyroid eye disease (TED) has had one or more symptoms of thyroid eye disease (TED). In some embodiments, one or more symptoms of one or more symptoms of thyroid eye disease (TED) are selected from the group consisting of lid retraction greater than 2 mm, exophthalmos (proptosis) of greater than or equal to 3 mm, Clinical Activity Score (CAS) from about 0 to about 7, and inconstant or constant diplopia. In some embodiments, a patient suffering from thyroid eye disease (TED) has had lid retraction greater than 2 mm. In some embodiments, a patient suffering from thyroid eye disease (TED) has had exophthalmos (proptosis) of greater than or equal to 3 mm. In some embodiments, a patient suffering from thyroid eye disease (TED) has had exophthalmos (proptosis) of greater than or equal to 3 mm above the normal range for their race and gender. In some embodiments, a patient suffering from thyroid eye disease (TED) has had Clinical Activity Score (CAS) from about 0 to about 7. In some embodiments, a patient suffering from thyroid eye disease (TED) has had inconstant or constant diplopia. In some embodiments, a patient suffering from thyroid eye disease (TED) has had inconstant diplopia. In some embodiments, a patient suffering from thyroid eye disease (TED) has had inconstant diplopia. In some embodiments, a patient exhibits fibrosis.

### Chronic Thyroid Eye Disease

Thyroid Eye Disease has historically been said to transition from an active and progressive phase (characterized by inflammation of orbital and external periorbital tissues) to a more stabilized and fibrotic, chronic phase. Active TED has been characterized by local inflammation of conjunctivae, superficial vasculature, orbital fat, lids, and extraocular muscles.

Active TED may be of variable duration and may last between 1-3 years. Chronic TED occurs when the autoimmune inflammation dampens, leaving sequelae of expanded, fibrotic orbital tissues and dysfunctional, tethered extraocular muscles, though evidence exists that even chronic TED patients may exhibit an underlying inflammatory component. Chronic TED may therefore be characterized either by the duration of symptoms (e.g., the time since first onset of symptoms) and/or by the severity of disease, e.g., based on one or more of the following criteria: (i) minor lid retraction <2 mm (mild) or lid retraction ≥2 mm (moderate to severe), (ii) mild soft tissue involvement (mild) or moderate/severe soft-tissue involvement (mod to severe), (iii) exophthalmos <3 mm above normal for race and gender (mild) or ≥3 mm above normal for race and gender (mod to severe), (iv) no or intermittent diplopia and corneal exposure responsive to lubricants (mild) or inconstant or constant diplopia (mod to severe); or sight-threatening dysthyroid optic neuropathy and or corneal breakdown (very severe). Further, chronic TED may be characterized by a fibrotic component to disease. However, a patient with chronic TED may still experience inflammatory episodes. Thus, a patient suffering from chronic TED may be further categorized based on the underlying disease activity into active chronic TED (e.g., Clinical Activity Score >=3) or inactive chronic TED (e.g., Clinical Activity Score <3).

In some embodiments, a patient suffers from chronic thyroid eye disease (TED). In some embodiments, a patient suffers from fibrosis associated with thyroid eye disease (TED).

In some embodiments, methods of treating a patient with chronic thyroid eye disease are provided. In some embodiments, a patient with chronic thyroid eye disease, prior to treatment, has had one or more symptoms for at least 12 months. In some embodiments, a patient with chronic thyroid eye disease, prior to treatment, has had one or more symptoms for at least 1 year. In some embodiments, a patient with chronic thyroid eye disease, prior to treatment, has had one or more symptoms for more than one year. In some embodiments, a patient with chronic thyroid eye disease, prior to treatment, has had one or more symptoms for at least 15 months. In some embodiments, a patient with chronic thyroid eye disease, prior to treatment, has had one or more symptoms for at least 2 years.

In some embodiments, a patient, prior to administration of a first dose of a pharmaceutical composition provided for herein, has had one or more symptoms of thyroid eye disease more than 2, 3, 4, 5, 6, or 7 years, or 1 to about 8 years, about 1 to about 7 years, about 1 to about 6 years, about 1 to about 5 years, about 1 to about 4 years, about 1 to about 3 years, about 1 to about 2 years, about 2 to about 8 years, about 2 to about 7 years, about 2 to about 6 years, about 2 to about 5 years, about 2 to about 4 years, about 2 to about 3 years, about 3 to about 8 years, about 3 to about 7 years, about 3 to about 5 years, about 3 to about 4 years, about 4 to about 8 years, about 4 to about 7 years, about 4 to about 6 years, about 4 to about 5 years, about 5 to about 8 years, about 5 to about 7 years, about 5 to about 6 years, about 6 to about 8 years, about 6 to about 7 years, or about 7 to about 8 years. In some embodiments, the patient, prior to administration a dose of a pharmaceutical composition provided for herein, has had one or more symptoms of thyroid eye disease no more than 63 months, 60 months, 48 months, 36 months, 24 months, or 12 or 13 months.

In some embodiments, a patient suffers from inactive chronic TED. In some embodiments, a patient suffering from inactive chronic TED, prior to treatment, had a Clinical Activity Score (CAS) of less than 2. In some embodiments, a patient suffering from inactive chronic TED, prior to treatment, had a Clinical Activity Score (CAS) 1. In some embodiments, a patient suffering from inactive chronic TED, prior to treatment, had a Clinical Activity Score (CAS) of 0 or 1.

In embodiments, a patient has a baseline CAS 1 and/or documented signs and symptoms that have persisted more than 12 months (one year) prior to commencement of treatment according to methods described herein. In embodiments, a patient has a baseline CAS 1 and documented signs and symptoms that have persisted more than 12 months (one year) prior to commencement of treatment according to methods described herein.

In embodiments, a patient has a baseline CAS 1 and/or documented signs and symptoms that have persisted more than 15 months prior to commencement of treatment according to methods described herein. In embodiments, a patient has a baseline CAS 1 and documented signs and symptoms that have persisted more than 15 months prior to commencement of treatment according to methods described herein.

In embodiments, a patient has a baseline CAS 1 and/or documented signs and symptoms that have persisted more than two years prior to commencement of treatment according to methods described herein. In embodiments, a patient has a baseline CAS ≤1 and documented signs and symptoms that have persisted more than two years prior to commencement of treatment according to methods described herein.

In some embodiments, a patient suffers from active chronic TED. In some embodiments, a patient suffering from inactive chronic TED, prior to treatment, had a Clinical Activity Score (CAS) of ≥2. In some embodiments, a patient suffering from inactive chronic TED, prior to treatment, had a Clinical Activity Score (CAS) ≥3. In some embodiments, a patient suffering from inactive chronic TED, prior to treatment, had a Clinical Activity Score (CAS) of ≥4.

In embodiments, a patient has a baseline CAS ≥2 and/or documented signs and symptoms that have persisted more than 12 months (one year) prior to commencement of treatment according to methods described herein. In embodiments, a patient has a baseline CAS ≥2 and documented signs and symptoms that have persisted more than 12 months (one year) prior to commencement of treatment according to methods described herein.

In embodiments, a patient has a baseline CAS ≥2 and/or documented signs and symptoms that have persisted more than 15 months prior to commencement of treatment according to methods described herein. In embodiments, a patient has a baseline CAS ≥2 and documented signs and symptoms that have persisted more than 15 months prior to commencement of treatment according to methods described herein. In embodiments, a patient has a baseline CAS ≥2 and/or documented signs and symptoms that have persisted more than two years prior to commencement of treatment according to methods described herein. In embodiments, a patient has a baseline CAS ≥2 and documented signs and symptoms that have persisted more than two years prior to commencement of treatment according to methods described herein.

### Methods of Treatment Using IGF-1R Inhibitors

The administration regimen depends on several factors, including the serum or tissue turnover rate of the therapeutic antibody, the level of symptoms, the immunogenicity of the therapeutic antibody, and the accessibility of the target cells in the biological matrix. Preferably, the administration regimen delivers sufficient therapeutic antibody to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602).

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent. In the case of human subjects, for example, chimeric, humanized and fully human antibodies may be desirable.

Antibodies or antigen binding fragments thereof, including any forms, variants, or derivatives thereof can be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. A total weekly dose is generally at least 0.05 g/kg body weight, more generally at least 0.2 g/kg, 0.5 g/kg, 1 g/kg, g/kg, 100 g/kg, 0.25 mg/kg, 1.0 mg/kg, 2.0 mg/kg, 5.0 mg/ml, 10 mg/kg, 25 mg/kg, 50 mg/kg or more (see, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144). Doses may also be provided to achieve a pre-determined target concentration of the antibody in the subject's serum, such as 0.1, 0.3, 1, 3, 10, 30, 100, 300 μg/ml or more. In other embodiments, a fully human antibody is administered subcutaneously or intravenously, on a weekly, biweekly, “every 4 weeks,” monthly, bimonthly, or quarterly basis at 10, 20, 50, 80, 100, 200, 500, 1000 or 2500 mg/subject or as otherwise provided for herein.

As used herein, “inhibit” or “treat” or “treatment” includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms including, for example, postponement or prevention in progression of the relevant medical condition. Thus, the terms denote that a beneficial result has been conferred on a vertebrate subject with a disorder, disease or symptom, or with the potential to develop such a disorder, disease or symptom.

As used herein, the terms “therapeutically effective amount”, “therapeutically effective dose” and “effective amount” refer to an amount of the antibody, or antigen binding fragment thereof, that, when administered alone or in combination with an additional therapeutic agent to a cell, tissue, or subject, is effective to cause a measurable improvement in one or more symptoms of a disease or condition or the progression of such disease or condition. A therapeutically effective dose further refers to that amount of the binding compound sufficient to result in at least partial amelioration of symptoms, e.g., treatment, healing, prevention or amelioration of the relevant medical condition, or an increase in rate of treatment, healing, prevention or amelioration of such conditions or prevention or postponement of progression of the relevant medical condition. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. In some embodiments, an effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity. In some embodiments, an amount is a therapeutically effective amount if it is an amount that can be used to treat or ameliorate a condition as provided for herein.

The term “subject” as used throughout includes any organism, such as an animal, including a mammal (e.g., rat, mouse, dog, cat, rabbit) and, for example, a human. A subject can be also be referred to as a patient. In some embodiments, the subject is a subject in need thereof. A subject that is “in need thereof” refers to a subject that has been identified as requiring treatment for the condition that is to be treated and is treated with the specific intent of treating such condition. The conditions can be, for example, any of the conditions described herein.

Whereas, an isolated antibody binds an epitope on an IGF-1R protein, or other protein described herein, and displays in vitro and/or in vivo IGF-1R inhibiting or therapeutic activities, the antibodies or antigen binding fragments thereof, capable of inhibiting IGF-1R function, are suitable both as therapeutic agents for treating IGF-1R-associated conditions in humans and animals. These conditions include thyroid-associated ophthalmopathy (TAO), also known as thyroid eye disease (TED), Graves' ophthalmopathy or orbitopathy (GO), thyrotoxic exophthalmos, dysthyroid ophthalmopathy, autoimmune associated eye disorders associated with IGF-1R signaling, inflammatory orbital disorder associated with IGF-1R signaling, and other thyroid eye disorders associated with IGF-1R signaling, including chronic TED. Accordingly, methods of treating such conditions are also provided, wherein the method comprises administering an antibody, or antigen binding fragment thereof, including any forms, variants, or derivatives thereof, to the subject with such a condition.

In some embodiments, the methods comprise administering a therapeutically or prophylactically effective amount of one or more monoclonal antibodies or antigen binding fragments of the antibodies described herein to a susceptible subject or to one exhibiting a condition in which IGF-1R is known or suspected to have caused the pathology observed. Any active form of the antibody can be administered, including, but not limited to scFV, Fab and F(ab′)2 fragments and other forms of antibodies provided for herein.

In some embodiments, the antibodies used are compatible with the recipient species such that the immune response to the antibodies does not result in an unacceptably short circulating half-life or induce an unacceptable immune response to the antibodies in the subject.

Treatment of individuals may comprise the administration of a therapeutically effective amount of the antibodies described herein. The antibodies can be provided in a kit, such as those provided herein. The antibodies can be used or administered alone or in admixture with another therapeutic, analgesic, or diagnostic agent, such as provided for herein. In providing a patient with an antibody, or fragment thereof, capable of binding to IGF-1R, or an antibody capable of protecting against IGF-1R, pathology in a recipient patient, the dosage of administered agent will vary depending upon such factors as the patient's age, weight, height, sex, general medical condition, previous medical history, etc.

An antibody, capable treating a condition associated with IGF-1R activity or use to treat an IGF-1R related pathology, is intended to be provided to subjects in an amount sufficient to affect a reduction, resolution, or amelioration in the IGF-1R related symptom or pathology. Such a pathology includes thyroid eye disease and the like.

Accordingly, in some embodiments, methods of treating a subject with an IGF-1R mediated disorder are provided. In some embodiments, the method comprises administering a pharmaceutical composition comprising an antibody, or antigen binding fragment thereof, including any forms, variants, or derivatives thereof, as provided herein. In some embodiments, the disorder is thyroid eye disease (TED). As provided for herein, the antibodies, or antigen binding fragments thereof, can be administered with other therapeutics. These can be administered simultaneously or sequentially.

In some embodiments, the antibodies, or antigen binding fragments thereof, may be used to treat thyroid eye disease (also referred to as thyroid-associated ophthalmopathy (TAO) or Graves' ophthalmopathy or orbitopathy (GO)). In some embodiments, the antibodies, or antigen binding fragments thereof, may be used to treating or reduce the severity of, thyroid-associated ophthalmopathy (TAO), or a symptom thereof. In some embodiments, methods comprise use of any forms, variants, or derivatives of the antibody or antigen binding fragment thereof.

In some embodiments, the antibodies, or antigen binding fragments thereof, may be used to treat thyrotoxic exophthalmos. In some embodiments, the antibodies, or antigen binding fragments thereof, may be used to treating or reduce the severity of, thyrotoxic exophthalmos, or a symptom thereof. In some embodiments, methods comprise use of any forms, variants, or derivatives of the antibody or antigen binding fragment thereof.

In some embodiments, the antibodies, or antigen binding fragments thereof, may be used to treat dysthyroid ophthalmopathy. In some embodiments, the antibodies, or antigen binding fragments thereof, may be used to treating or reduce the severity of, dysthyroid ophthalmopathy, or a symptom thereof. In some embodiments, methods comprise use of any forms, variants, or derivatives of the antibody or antigen binding fragment thereof.

In some embodiments, the antibodies, or antigen binding fragments thereof, may be used to treat autoimmune associated eye disorders associated with IGF-1R signaling. In some embodiments, the antibodies, or antigen binding fragments thereof, may be used to treat or reduce the severity of, autoimmune associated eye disorders associated with IGF-1R signaling, or a symptom thereof. In some embodiments, methods comprise use of any forms, variants, or derivatives of the antibody or antigen binding fragment thereof.

In some embodiments, the antibodies, or antigen binding fragments thereof, may be used to treat inflammatory orbital disorder associated with IGF-1R signaling. In some embodiments, the antibodies, or antigen binding fragments thereof, may be used to treating or reduce the severity of, inflammatory orbital disorder associated with IGF-1R signaling, or a symptom thereof. In some embodiments, methods comprise use of any forms, variants, or derivatives of the antibody or antigen binding fragment thereof.

In some embodiments, the antibodies, or antigen binding fragments thereof, may be used to treat thyroid eye disorders associated with IGF-1R signaling, including chronic TED. In some embodiments, the antibodies, or antigen binding fragments thereof, may be used to treat or reduce the severity of, thyroid eye disorders associated with IGF-1R signaling (including chronic TED), or a symptom thereof. In some embodiments, methods comprise use of any forms, variants, or derivatives of the antibody or antigen binding fragment thereof.

In some embodiments, methods or uses are provided to reduce proptosis in an eye in a subject with thyroid-associated ophthalmopathy (TAO).

In some embodiments, the subject is a subject how has previously been treated with a different antibody than those provided herein.

In some embodiments, methods or uses are provided to Clinical Activity Score (CAS) in subject who has or is suspected of having thyroid-associated ophthalmopathy (TAO).

In some embodiments, methods or uses are provided to reduce proptosis by at least 2 mm. In some embodiments, methods or uses are provided to reduce proptosis by at least 3 mm. In some embodiments, methods or uses are provided to reduce proptosis by at least 2-3 mm or 2-4 mm. In some embodiments, the proptosis is reduced by at least 2, 3, or 4 mm. In some embodiments, the reduction in proptosis is seen within 3 weeks of the first dose administration. In some embodiments, the reduction in proptosis is seen within 6 weeks of the first dose administration.

In some embodiments, the subject has a reduced the clinical activity score (CAS) in a subject with thyroid-associated ophthalmopathy (TAO).

In some embodiments, the subject who is treated has the proptosis is reduced by at least 2 mm. In some embodiments, the subject who is treated has the proptosis is reduced by at least 3 mm. In some embodiments, the subject who is treated has the proptosis is reduced by at least 4 mm.

In some embodiments, in the subjects who are treated the clinical activity score (CAS) of the subject is reduced by at least 2 points. In some embodiments, the clinical activity score (CAS) of the subject is reduced to one (1). In some embodiments, the clinical activity score (CAS) of the subject is reduced to zero (0).

In some embodiments, methods off treating or reducing the severity of thyroid-associated ophthalmopathy (TAO) in a subject are provided, wherein the treatment with said antibody (i) reduces proptosis by at least 2 mm in an eye; (ii) is not accompanied by a deterioration of 2 mm or more in the other (or fellow eye); and (iii) reduces the CAS in said subject to either one (1) or zero (0).

In some embodiments, methods of improving the quality of life in a subject with thyroid-associated ophthalmopathy (TAO, also called Graves' Ophthalmopathy/Graves' Orbitopathy) are provided. In some embodiments, the quality of life is measured by the Graves' Ophthalmopathy Quality of Life (GO-QoL) assessment, or either the Visual Functioning or Appearance subscale thereof. In some embodiments, the treatment results in an improvement of greater than or equal to 8 points on the GO-QoL. In some embodiments, the treatment results in an improvement on the Functioning subscale of the GO-QoL. In some embodiments, the treatment results in an improvement on the Appearance subscale of the GO-QoL.

In some embodiments, methods of treating or reducing the severity of diplopia in a subject with thyroid-associated ophthalmopathy (TAO) are provided. In some embodiments, the diplopia is constant diplopia. In some embodiments, the diplopia is inconstant diplopia. In some embodiments, the diplopia is intermittent diplopia. In some embodiments, the improvement in or reduction in severity of diplopia is sustained at least 20 weeks after discontinuation of antibody administration. In some embodiments, the improvement in or reduction in severity of diplopia is sustained at least 50 weeks after discontinuation of antibody administration. In some embodiments, the diplopia is improved in the subject within 3 weeks or within 6 weeks of the first dose.

In some embodiments, a patient can be characterized by Graves Ophthalmopathy Quality of Life (GO-QoL) score. In addition to proptosis (or exophthalmos) and CAS, quality of life is also evaluated with the use of the GO quality of life (GO-QoL) questionnaire. This questionnaire is designed to determine the improved quality of life after treatment with a method disclosed herein. In some embodiments, questionnaire may determine the decreased or lack of side effects after being treated with an antibody, or an antigen binding fragment thereof, according to a method disclosed herein as compared to treatment with glucocorticoids. The GO-QoL is a 16-item self-administered questionnaire divided into 2 subsets and used to assess the perceived effects of TED by the subjects on (i) their daily physical activity as it relates to visual function, and (ii) psychosocial functioning. Quality of life is evaluated with the use of the GO QoL questionnaire. The GO-QoL questionnaire [C. B. Terwee et al, 1998] is completed on Day 1 and Weeks 6, 12, and 24 (or PW) during the Treatment Period, and at Months 7 and 12 (or PW) during the Follow-Up Period. The GO-QoL is a 16-item self-administered questionnaire divided into two self-assessment subscales; one covering impact of visual function on daily activities, the other assesses the impact of self-perceived appearance. The visual function subscale covers activities such as driving, walking outdoors, reading, and watching television. The appearance subscale asks the subject questions such as whether ophthalmopathy has altered the subject's appearance, caused other people to have a negative reaction to the subject, caused social isolation, and caused the subject to try to mask his or her appearance. Each subscale has 8 questions which are answered with: yes—very much so; yes—a little; or no—not at all. Each question is scored 0-2, respectively, and the total raw score is then mathematically transformed to a 0-100 scale, where 0 represents the most negative impact on quality of life, and 100 represents no impact. A change of > or greater than equal to 8 points on the 0-100 scale has been shown to be clinically meaningful. The combined score takes raw scores from both subscales and again transforms them to a single 0-100 scale. The questionnaire has two self-assessment subscales. Each subscale has 8 questions which are answered with: (i) yes—very much so; (ii) yes—a little; or (iii) no—not at all. Each question is scored 0-2, respectively, and the total raw score is then mathematically transformed to a 0-100 scale, where 0 represents the most negative impact on quality of life, and 100 represents no impact. A change of >8 points on the 0-100 scale is considered to be clinically meaningful. The combined score takes raw scores from both subscales and again transforms them to a single 0-100 scale.

Patients can also be assessed by the presence of absence of Gorman Grading of Diplopia. The Gorman assessment of subjective diplopia includes four categories: no diplopia (absent), diplopia when the patient is tired or awakening (intermittent), diplopia at extremes of gaze (inconstant), and continuous diplopia in the primary or reading position (constant). Patients are scored according to which grade of diplopia they are experiencing. An improvement of greater than equal or to 1 grade is considered clinically meaningful.

### Fibrosis

In some embodiments, methods described herein can treat fibrosis (e.g., ocular fibrosis). In some embodiments, a therapeutic benefit is an alleviation of fibrosis (e.g., ocular fibrosis). In some embodiments, a therapeutic benefit is a reversal in fibrosis (e.g., ocular fibrosis). In some embodiments, a therapeutic benefit is prevention of fibrosis or prevention of further fibrosis (e.g., ocular fibrosis). In some embodiments, a therapeutic benefit from alleviation and/or reversal in ocular fibrosis is improvement in vision. In some embodiments, a therapeutic benefit from alleviation and/or reversal in ocular fibrosis is no further deterioration in vision or no worsening of symptoms (e.g., preservation of vision or no further vision loss or impairment). In embodiments, treatment of ocular fibrosis results in improvements in diplopia and/or proptosis (e.g., as described herein). In embodiments, the fibrosis (e.g., ocular fibrosis) is associated with or results from inflammation. In embodiments, the fibrosis (e.g., ocular fibrosis) is associated with or results from an autoimmune disorder. In embodiments, the fibrosis (e.g., ocular fibrosis) is associated with or results from thyroid eye disease. In embodiments, ocular fibrosis is fibrosis that develops around the extraocular muscles. embodiments, the fibrosis (e.g., ocular fibrosis) follows an inflammatory period of TED (e.g., fibrosis following active TED or following an inflammatory flare-up in a patient having chronic and/or inactive TED). One skilled in the art will appreciate that a variety of methods well-known in the art nay be used to assess changes in fibrosis (e.g., ocular fibrosis), including biopsy followed by staining or via imaging techniques (e.g., computed tomography (CT) or magnetic resonance imaging (MRI)). Still other methods of diagnosing and/or characterizing fibrosis (e.g., ocular fibrosis) include dye-based angiography, optical coherence tomography (OCT), and OCT angiography (OCTA). Fibrosis may also be assessed using levels of inflammatory or fibrotic factors. In some embodiments, the fibrosis is assessed through one or more of analysis of adenosine monophosphate-activated protein kinase (AMPK), fibronectin, alpha-SMA, and collagen staining.

### Therapeutically Effective Dosing Regimens

In some embodiments, a method comprises administering an IGF-1R inhibitor (e.g., an antibody, such as those provided herein) to a patient at a therapeutically effective dosing regimen. Exemplary dosage amounts and therapeutically effective dosing regimens are described herein.

In embodiments, an antibody is administered at a dose of about 3.0-20.0 mg/kg (e.g., at a dose of about 3.0-10.0 or about 10.0-20.0 mg/kg). In embodiments, an antibody is administered at a dose of about 3.0-10.0 mg/kg. In embodiments, an antibody is administered at a dose of about 3.0-20.0 mg/kg. In embodiments, an antibody is administered at a dose of about 10.0-20.0 mg/kg.

In embodiments, an antibody is administered at a dose of about 3.0 mg/kg, about 5.0 mg/kg, about 10 mg/kg, or about 20 mg/kg. In embodiments, an antibody is administered at a dose of about 3.0 mg/kg. In embodiments, an antibody is administered at a dose of about 5.0 mg/kg. In embodiments, an antibody is administered at a dose of about 10 mg/kg. In embodiments, an antibody is administered at a dose of about 20 mg/kg.

In embodiments, an antibody is administered at a dose of at least about 3.0 mg/kg, about 5.0 mg/kg, about 10 mg/kg, or about 20 mg/kg. In embodiments, an antibody is administered at a dose of at least about 3.0 mg/kg. In embodiments, an antibody is administered at a dose of at least about 5.0 mg/kg. In embodiments, an antibody is administered at a dose of at least about 10 mg/kg. In embodiments, an antibody is administered at a dose of at least 20 mg/kg.

In embodiments, an antibody is administered at a dose of no more than about 3.0 mg/kg, about 5.0 mg/kg, about 10 mg/kg, or about 20 mg/kg. In embodiments, an antibody is administered at a dose of no more than about 3.0 mg/kg. In embodiments, an antibody is administered at a dose of no more than about 5.0 mg/kg. In embodiments, an antibody is administered at a dose of no more than about 10 mg/kg. In embodiments, an antibody is administered at a dose of no more than about 20 mg/kg.

In some embodiments, a pharmaceutical composition comprising the antibody is administered at a dose of about 10 mg/kg. In some embodiments, the pharmaceutical composition administered at a dose of about 10 mg/kg is administered to the patient every 21 days.

In some embodiments, the pharmaceutical composition is administered at a dose of about 10 mg/kg is administered to the patient every 21 days for a total of about 4 to about 8 doses, about 4, about 5, about 6, about 7, or about 8 doses.

In some embodiments, a pharmaceutical composition comprising the antibody is administered at a dose of about 3 mg/kg. In some embodiments, the pharmaceutical composition administered at a dose of about 3 mg/kg is administered to the patient every 21 days.

In some embodiments, the pharmaceutical composition is administered at a dose of about 3 mg/kg is administered to the patient every 21 days for a total of about 4 to about 8 doses, about 4, about 5, about 6, about 7, or about 8 doses.

In embodiments, a therapeutically effective dosage regimen comprises administration of one or more doses to a patient (e.g., one or more doses of an antibody as described herein).

In embodiments, a therapeutically effective dosage regimen comprises administration of a first dose (e.g., any dose amount of an antibody described herein) to a patient.

In embodiments, a therapeutically effective dosage regimen comprises administration of subsequent dose(s) (e.g., any dose amount of an antibody described herein) to a patient. In embodiments, a first dose is the same amount as a subsequent dose. In embodiments, a first dose is different amount as a subsequent dose. In embodiments, a first dose is a higher amount than a subsequent dose. In embodiments, a first dose is a lower amount than a subsequent dose.

In embodiments, a subsequent dose (e.g., of an antibody as described herein) is administered to the patient once every 2-6 weeks. In embodiments, a subsequent dose (e.g., of an antibody as described herein) is administered to the patient once every two weeks, once every three weeks, once every four weeks, once every month, once every five weeks, or once every six weeks. In embodiments, a subsequent dose (e.g., of an antibody as described herein) is administered to the patient once every two weeks. In embodiments, a subsequent dose (e.g., of an antibody as described herein) is administered to the patient once every three weeks. In embodiments, a subsequent dose (e.g., of an antibody as described herein) is administered to the patient once every four weeks. In embodiments, a subsequent dose (e.g., of an antibody as described herein) is administered to the patient once every month. In embodiments, a subsequent dose (e.g., of an antibody as described herein) is administered to the patient once every five weeks.

In embodiments, a subsequent dose (e.g., of an antibody as described herein) is administered to the patient once every six weeks.

In some embodiments, the method comprises administering a dose to the patient about every 21 days. In some embodiments, the patient is administered at least 5 consecutive doses of the pharmaceutical composition, wherein the dose is administered about every 21 days. In some embodiments, the dose is administered every 21 days. In some embodiments, the patient is administered at least 5 consecutive doses of the pharmaceutical composition, wherein the dose is administered about every 21 days or 21 days. In some embodiments, the patient is administered at least 8 consecutive doses of the pharmaceutical composition, wherein the dose is administered about every 21 days, which includes every 21 days.

In embodiments, a patient is administered at least 3-10 subsequent doses. In embodiments, a patient is administered at least 3, 4, 5, 6, 7, 8, 9 or 10 subsequent doses. In embodiments, a patient is administered at least 3 subsequent doses. In embodiments, a patient is administered at least 4 subsequent doses. In embodiments, a patient is administered at least 5 subsequent doses. In embodiments, a patient is administered at least 6 subsequent doses. In embodiments, a patient is administered at least 7 subsequent doses. In embodiments, a patient is administered at least 8 subsequent doses. In embodiments, a patient is administered at least 9 subsequent doses. In embodiments, a patient is administered at least 10 subsequent doses.

In embodiments, a patient is administered 3-10 subsequent doses. In embodiments, a patient is administered 3, 4, 5, 6, 7, 8, 9 or 10 subsequent doses. In embodiments, a patient is administered 3 subsequent doses. In embodiments, a patient is administered 4 subsequent doses. In embodiments, a patient is administered 5 subsequent doses. In embodiments, a patient is administered 6 subsequent doses. In embodiments, a patient is administered 7 subsequent doses. In embodiments, a patient is administered 8 subsequent doses. In embodiments, a patient is administered 9 subsequent doses. In embodiments, a patient is administered 10 subsequent doses.

In embodiments, at least 3-10 total doses (e.g., of an antibody described herein such as 5-10 or 5-8 total doses of an antibody described herein) are administered to a patient. In embodiments, at least 3, 4, 5, 6, 7, 8, 9 or 10 total doses (e.g., of an antibody as described herein) are administered to a patient. In embodiments, at least 4-8 total doses (e.g., of an antibody as described herein) are administered to a patient. In embodiments, at least 5-8 total doses (e.g., of an antibody as described herein) are administered to a patient. In embodiments, no more than 4, 5, 6, 7, 8, 9 or 10 total doses (e.g., of an antibody as described herein) are administered to a patient.

In embodiments, 3, 4, 5, 6, 7, 8, 9 or 10 total doses (e.g., of an antibody as described herein) are administered to a patient. In embodiments, no more than 5, 6, 7, 8, 9 or 10 total doses (e.g., of an antibody as described herein) are administered to a patient. In embodiments, 3, 4, 5, 6, 7, 8, 9 or 10 total doses (e.g., of an antibody as described herein) are administered to a patient. In embodiments, no more than 4-8 total doses (e.g., of an antibody as described herein) are administered to a patient. In embodiments, no more than 5-8 total doses (e.g., of an antibody as described herein) are administered to a patient. In embodiments, 4-8 total doses (e.g., of an antibody as described herein) are administered to a patient. In embodiments, 5-8 total doses (e.g., of an antibody as described herein) are administered to a patient. In embodiments, 5 total doses (e.g., of an antibody as described herein) are administered to a patient. In embodiments, 8 total doses (e.g., of an antibody as described herein) are administered to a patient.

In embodiments, a therapeutically effective regimen comprises administration of a first dose (e.g., of an antibody as described herein) and 3-7 subsequent doses (e.g., of an antibody as described herein) such that 4-8 total doses (e.g., of an antibody as described herein) are administered a patient. In embodiments, a therapeutically effective regimen comprises administration of a first dose (e.g., of an antibody as described herein) and 4-7 subsequent doses (e.g., of an antibody as described herein) such that 5-8 total doses (e.g., of an antibody as described herein) are administered a patient. In embodiments, a first dose is 3.0 mg/kg, 5.0 mg/kg, 10 mg/kg, or 20 mg/kg and/or a subsequent dose (e.g., each of 3-7 subsequent doses) is 3.0 mg/kg, 5.0 mg/kg, 10 mg/kg, or 20 mg/kg. In embodiments, a first dose is 3.0 mg/kg, 5.0 mg/kg, 10 mg/kg, or 20 mg/kg and/or a subsequent dose (e.g., each of 4-7 subsequent doses) is 3.0 mg/kg, 5.0 mg/kg, 10 mg/kg, or 20 mg/kg. In embodiments, a first dose of an antibody described herein is 10 mg/kg and/or a subsequent dose (e.g., each of 3-7 subsequent doses) of an antibody described herein is 10 mg/kg or 20 mg/kg. In embodiments, a first dose of an antibody described herein is 10 mg/kg and/or a subsequent dose (e.g., each of 4-7 subsequent doses) of an antibody described herein is 10 mg/kg or 20 mg/kg.

In some embodiments, the antibody is administered at a dose of 1 mg/kg to about 5 mg/kg antibody as a first dose. In some embodiments, the antibody is administered at a dose of 5 mg/kg to about 10 mg/kg antibody as a first dose. In some embodiments, the antibody is administered at a dose of 5 mg/kg to 20 mg/kg antibody in subsequent doses. In some embodiments, the antibody is administered in the following amounts: 10 mg/kg antibody as a first dose; and 10 mg/kg antibody in subsequent doses. In some embodiments, the antibody is administered in the following amounts: 10 mg/kg antibody as a first dose; and 20 mg/kg antibody in subsequent doses.

In embodiments, a first dose of an antibody described herein is 10 mg/kg and a subsequent dose (e.g., each of 3-7 subsequent doses) of an antibody described herein is 10 mg/kg. In embodiments, a first dose of an antibody described herein is 10 mg/kg and three subsequent doses are each 10 mg/kg.

In embodiments, a first dose of an antibody described herein is 10 mg/kg and a subsequent dose (e.g., each of 4-7 subsequent doses) of an antibody described herein is 10 mg/kg. In embodiments, a first dose of an antibody described herein is 10 mg/kg and four subsequent doses are each 10 mg/kg.

In embodiments, a first dose of an antibody described herein is 10 mg/kg and a subsequent dose (e.g., each of 4-7 subsequent doses) of an antibody described herein is 20 mg/kg. In embodiments, a first dose of an antibody described herein is 10 mg/kg and seven subsequent doses are each 20 mg/kg. In some embodiments, the subsequent doses are administered every three weeks for at least 12 weeks.

Doses described herein can be administered according to methods known in the art. Exemplary routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial. In some embodiments, the subsequent doses are administered every three weeks for at least 12 weeks. In some embodiments, the subsequent doses are administered every three weeks for at least 21 weeks.

In embodiments, a dose is administered by infusion, intravenously, or subcutaneously. In embodiments, a dose is administered intravenously, such as by infusion.

In embodiments, the method comprises intravenously administering a dose of 10 mg/kg of an anti-IGF-1R antibody to the subject at a regular interval for a period sufficient to reduce one or more symptoms associated with thyroid eye disease (e.g., chronic thyroid eye disease), wherein the anti-IGF-1R antibody comprises a heavy chain comprising a HCDR1 of SEQ ID NO: 7, a HCDR2 of SEQ ID NO: 8, and a HCDR3 of SEQ ID NO: 9 and a light chain comprising a LCDR1 of SEQ ID NO: 4, a LCDR2 of SEQ ID NO: 5 and a LCDR3 of SEQ ID NO: 6. In some embodiments, the anti-IGF-1R antibody comprises a light chain and a heavy chain, wherein the light chain comprises a variable region having the amino acid sequence of SEQ ID NO: 2 and the heavy chain comprises a variable region having the amino acid sequence of SEQ ID NO: 3. In some embodiments, the light chain comprises an amino acid sequence of SEQ ID NO: 11. In some embodiments, the heavy chain comprises an amino acid sequence of SEQ ID NO: 10. In some embodiments, the heavy chain comprises an amino acid sequence of SEQ ID NO: 10 and the light chain comprises an amino acid sequence of SEQ ID NO: 11. In some embodiments, the anti-IGF-1R antibody is administered by intravenous infusion. In some embodiments, the anti-IGF-1R antibody is administered every 3 weeks. In some embodiments, the anti-IGF-1R antibody is administered for a period sufficient for 4 doses. In some embodiments, the anti-IGF-1R antibody is administered for a period sufficient for 5 doses. In some embodiments, the anti-IGF-1R antibody is administered for a period sufficient for 8 doses. In some embodiments, the anti-IGF-1R antibody is administered for a period selected from 3 weeks, 6 weeks, 9 weeks, 12 weeks, 15 weeks, 18 weeks, 21 weeks, 24 weeks or longer.

### Efficacy

Methods described herein can provide therapeutic benefits to a patient. In embodiments, the therapeutic benefits are assessed relative to a baseline (e.g., as assessed prior to beginning a therapeutically effective dosing regimen as described herein). Exemplary, non-limiting therapeutic effects of the claimed methods include treatment of exophthalmos (proptosis), treatment of diplopia, and/or reduction in a Clinical Activity Score (CAS).

In embodiments, a therapeutic benefit is observed for one or more of the following: proptosis, CAS, extraocular muscle volume, orbital fat volume, manual measurement of lid retraction, Graves' Orbitopathy-Quality of Life (GO-QoL) combined score, GO-QoL activity subscale, GO-QoL appearance subscale, visual acuity (VA), and Gorman Subjective Diplopia Score (Diplopia Score).

In embodiments, extent of thyroid eye disease (e.g., proptosis) is relative to the normal range for their race and gender.

In embodiments, a patient has one or more of the following symptoms of thyroid eye disease: lid retraction greater than 2 mm; exophthalmos (proptosis) of greater than or equal to 3 mm; Clinical Activity Score (CAS) from about 0 to about 7; inconstant or constant diplopia, or any combination thereof.

In some embodiments, the exophthalmos (proptosis) is greater than or equal to 3 mm above the normal range for their race and gender.

In embodiments, methods described herein provide a therapeutic benefit for one or more of the components of CAS.

In embodiments, a patient has a reduction of proptosis of ≥2 mm from baseline (e.g., in the most proptotic eye). In embodiments, a reduction is measured by exophthalmometry or by MRICT. In embodiments, a reduction is determined at fifteen weeks from baseline. In embodiments, fifteen weeks from baseline is also three weeks after a fifth total dose in the dosage regimen. In embodiments, a patient has a reduction of proptosis of 2 mm from baseline in the most proptotic eye. In embodiments a reduction of proptosis in the most proptotic eye occurs with without a corresponding increase of ≥2 mm in the other eye.

In embodiments, administration of the therapeutically effective dosing regimen results in reduction of the proptosis by about 1 to about 3 mm, about 1 to about 2 mm, about 2 to about 3 mm from baseline (e.g., as measured by exophthalmometry or by MRI/CT).

In some embodiments, a patient with chronic TED and a CAS of 0 or 1 has a reduction in proptosis from about −1 mm to about −2 mm. In some embodiments, a patient with chronic TED and a CAS of 0 or 1 has a reduction in proptosis from about −1.2 mm to about −2 mm.

In some embodiments, a patient with chronic TED and a CAS of 0 or 1 has a reduction in proptosis from about −1.3 mm to about −2 mm. In some embodiments, a patient with chronic TED and a CAS of 0 or 1 has a reduction in proptosis from about −1.4 mm to about −2 mm. In some embodiments, a patient with chronic TED and a CAS of 0 or 1 has a reduction in proptosis from about −1.5 mm to about −2 mm. In some embodiments, a patient with chronic TED and a CAS of 0 or 1 has a reduction in proptosis from about −1.3 mm to about −1.8 mm. In some embodiments, a patient with chronic TED and a CAS of 0 or 1 has a reduction in proptosis of about −1.1 mm, −1.2 mm, −1.3 mm, −1.4 mm, −1.5 mm, −1.6 mm, −1.7 mm, −1.8 mm, −1.9 mm, or −2.0 mm. In some embodiments, the reduction occurs within 6 weeks of the administration of the first dose. In some embodiments, the reduction occurs after 2 doses are administered to the patient. In some embodiments, the dose is about 3 mg/kg or 10 mg/kg, or other doses as provided for herein. In some embodiments, each of the doses that are administered to the patient are the same.

In embodiments, administration of the therapeutically effective dosing regimen results in reduction of the proptosis by about 1 to about 3 mm, about 1 to about 2 mm, about 2 to about 3 mm from baseline within 6 weeks of the first dose (e.g., as measured by exophthalmometry or by MRI/CT). In embodiments, administration of the therapeutically effective dosing regimen results in reduction of the proptosis by about 1 to about 3 mm from baseline within 6 weeks of the first dose (e.g., as measured by exophthalmometry or by MRI/CT). In embodiments, administration of the therapeutically effective dosing regimen results in reduction of the proptosis by about 1 to about 2 mm from baseline within 6 weeks of the first dose (e.g., as measured by exophthalmometry or by MRI/CT). In embodiments, administration of the therapeutically effective dosing regimen results in reduction of the proptosis by about 2 to about 3 mm from baseline within 6 weeks of the first dose (e.g., as measured by exophthalmometry or by MRI/CT).

In embodiments, administration of the therapeutically effective dosing regimen results in reduction of the proptosis by about 1 to about 3 mm, about 1 to about 2 mm, about 2 to about 3 mm from baseline within 6 weeks of the first dose as measured by exophthalmometry or by MRICT. In embodiments, proptosis is reduced by about 2-3 mm from baseline within 6 weeks of the first dose as measured by exophthalmometry or by MRI/CT.

In some embodiments, the proptosis in the subject with chronic thyroid eye disease is reduced by about 1 to about 3 mm, about 1 to about 2 mm, about 2 to about 3 mm from baseline as measured by exophthalmometry or by MRICT. In some embodiments, the propostis is reduced by about 2-3 mm from baseline as measured by exophthalmometry or by MRI/CT. In some embodiments, the reduction is seen within 6 or at 6 weeks after administration of the first dose. In some embodiments, the reduction is seen after 2 doses are administered. In some embodiments, the dose is about 3 mg/kg or 10 mg/kg, or other doses as provided for herein. In some embodiments, each of the doses that are administered to the patient are the same.

In embodiments, administration of the therapeutically effective dosing regimen results in a therapeutic benefit that is no worsening in CAS from baseline. In embodiments, the non-worsening occurs without a corresponding increase of ≥2 points in the other eye. In embodiments, administration of the therapeutically effective dosing regimen results in a decrease in CAS from baseline (e.g., a decrease or 1, 2, or 3 in CAS). In embodiments, the decrease occurs without a corresponding increase of ≥2 points in the other eye. In embodiments, administration of the therapeutically effective dosing regimen results in a therapeutic benefit that is no worsening in CAS from baseline, and wherein the baseline CAS is 0 or 1. In embodiments, administration of the therapeutically effective dosing regimen results in a therapeutic benefit that is a CAS of 0. In embodiments, administration of the therapeutically effective dosing regimen results in a therapeutic benefit that is a CAS of 1. In embodiments, administration of the therapeutically effective dosing regimen results in a therapeutic benefit that is a decrease of CAS of ≥2 from baseline.

In embodiments, administration of the therapeutically effective dosing regimen results in a therapeutic benefit that is treatment of diplopia. In embodiments, treatment of diplopia is assessed using a change in the Gorman Subjective Diplopia Score (Diplopia Score), which ranges from 0 to 3 and includes four categories:
no diplopia (absent, scored as 0),
diplopia in the primary position of gaze when the participant is tired or awakening (intermittent, scored as 1),
diplopia at extremes of gaze (inconstant, scored as 2),
continuous diplopia in the primary or reading position (constant, scored as 3).

In embodiments, a patient having a baseline Diplopia Score >0 has a reduction in Diplopia Score to 0 after commencing treatment according to methods described herein.

In embodiments, a therapeutic benefit is a change of 8 points or greater on the combined score on the 0-100 scale of the Graves' Orbitopathy-Quality of Life (GO-QoL) questionnaire or on an individual subscale (e.g., the GO-QoL activity subscale or the GO-QoL appearance subscale).

In embodiments, a therapeutic benefit is observed for one or more of the following: extraocular muscle volume, orbital fat volume, manual measurement of lid retraction, and visual acuity (VA).

In some embodiments, the patient achieves an improvement in one or more parameters selected from: proptosis, CAS, extraocular muscle volume, orbital fat volume, manual measurement of lid retraction, Graves' Orbitopathy-Quality of Life (GO-QoL) combined score, GO-QoL activity subscale, GO-QoL appearance subscale, visual acuity, Gorman Subjective Diplopia Score, and EQ-5D-5L QoL questionnaire. In some embodiments, the patient achieves an improvement in at least two or more parameters. In some embodiments, the patient achieves an improvement in at least three or more parameters. In some embodiments, the patient achieves an improvement in at least four or more parameters.

In embodiments, a therapeutic benefit (e.g., in exophthalmos (proptosis), CAS, and/or diplopia as described herein) results within 3-52 weeks of commencing treatment (at baseline). In embodiments, a therapeutic benefit (e.g., in exophthalmos (proptosis), CAS, and/or diplopia as described herein) results within 6, 9, 12, 15, 18, 21, 24, 36, or 52 weeks of commencing treatment (at baseline). In embodiments, a therapeutic benefit (e.g., in exophthalmos (proptosis), CAS, and/or diplopia as described herein) results at 6, 9, 12, 15, 18, 21, 24, 36, or 52 weeks of commencing treatment (at baseline). In embodiments, a therapeutic benefit (e.g., in exophthalmos (proptosis), CAS, and/or diplopia as described herein) results within or at 6 weeks of commencing treatment (at baseline). In embodiments, a therapeutic benefit (e.g., in exophthalmos (proptosis), CAS, and/or diplopia as described herein) results within or at 9 weeks of commencing treatment (at baseline). In embodiments, a therapeutic benefit (e.g., in exophthalmos (proptosis), CAS, and/or diplopia as described herein) results within or at 12 weeks of commencing treatment (at baseline). In embodiments, a therapeutic benefit (e.g., in exophthalmos (proptosis), CAS, and/or diplopia as described herein) results within or at 15 weeks of commencing treatment (at baseline). In embodiments, a therapeutic benefit (e.g., in exophthalmos (proptosis), CAS, and/or diplopia as described herein) results within or at 18 weeks of commencing treatment (at baseline). In embodiments, a therapeutic benefit (e.g., in exophthalmos (proptosis), CAS, and/or diplopia as described herein) results within or at 21 weeks of commencing treatment (at baseline). In embodiments, a therapeutic benefit (e.g., in exophthalmos (proptosis), CAS, and/or diplopia as described herein) results within or at 24 weeks of commencing treatment (at baseline). In embodiments, a therapeutic benefit (e.g., in exophthalmos (proptosis), CAS, and/or diplopia as described herein) results within or at 36 weeks of commencing treatment (at baseline). In embodiments, a therapeutic benefit (e.g., in exophthalmos (proptosis), CAS, and/or diplopia as described herein) results within or at 52 weeks of commencing treatment (at baseline).

In embodiments, a therapeutic benefit (e.g., in exophthalmos (proptosis), CAS, and/or diplopia as described herein) is present after the first dose. In embodiments, a therapeutic benefit (e.g., in exophthalmos (proptosis), CAS, and/or diplopia as described herein) results after a subsequent dose. In embodiments, a therapeutic benefit (e.g., in exophthalmos (proptosis), CAS, and/or diplopia as described herein) is present after a second total dose. In embodiments, a therapeutic benefit (e.g., in exophthalmos (proptosis), CAS, and/or diplopia as described herein) is present after a third total dose. In embodiments, a therapeutic benefit (e.g., in exophthalmos (proptosis), CAS, and/or diplopia as described herein) is present after a fourth total dose. In embodiments, a therapeutic benefit (e.g., in exophthalmos (proptosis), CAS, and/or diplopia as described herein) is present after a fifth total dose. In embodiments, a therapeutic benefit (e.g., in exophthalmos (proptosis), CAS, and/or diplopia as described herein) is present after a sixth total dose. In embodiments, a therapeutic benefit (e.g., in exophthalmos (proptosis), CAS, and/or diplopia as described herein) is present after a seventh total dose. In embodiments, a therapeutic benefit (e.g., in exophthalmos (proptosis), CAS, and/or diplopia as described herein) is present after an eighth total dose.

In embodiments, a therapeutic benefit (e.g., in exophthalmos (proptosis), CAS, and/or diplopia as described herein) is present after a last dose in a therapeutically effective dosage regiment. In embodiments, a last dose is a fourth total dose. In embodiments, a last dose is a fifth total dose. In embodiments, a last dose is a sixth total dose. In embodiments, a last dose is a seventh total dose. In embodiments, a last dose is an eighth total dose. In embodiments, a therapeutic benefit is present at fifteen weeks after baseline.

In embodiments, a therapeutic benefit (e.g., in exophthalmos (proptosis), CAS, and/or diplopia as described herein) is present at or after nine weeks from baseline.

In embodiments, a therapeutic benefit (e.g., in exophthalmos (proptosis), CAS, and/or diplopia as described herein) is present at or after twelve weeks from baseline.

In embodiments, a therapeutic benefit (e.g., in exophthalmos (proptosis), CAS, and/or diplopia as described herein) is present at or after fifteen weeks from baseline.

In embodiments, a therapeutic benefit (e.g., in exophthalmos (proptosis), CAS, and/or diplopia as described herein) is present at or after twenty-one weeks from baseline.

In embodiments, a therapeutic benefit (e.g., in exophthalmos (proptosis), CAS, and/or diplopia as described herein) is present at or after twenty-four weeks from baseline.

In some embodiments, after first dose of the antibody, the clinical activity score of the subject is reduced. In some embodiments, after two doses of the antibody, the clinical activity score of the subject is reduced.

In some embodiments, the subject with chronic thyroid eye disease does not have any significant adverse events after administration of the antibody. In some embodiments, the subject with chronic thyroid eye disease does not have any significant adverse events caused by administration of the antibody. In some embodiments, the patient does not have any hearing impairment, ototoxic changes in audiometry, or hyperglycemic events after administration of the pharmaceutical composition. In some embodiments, the patient does not have any hearing impairment, ototoxic changes in audiometry, or hyperglycemic events caused by the administration of the pharmaceutical composition.

In some embodiments, the patient does not have any IV infusion associated adverse events after administration of the pharmaceutical composition. In some embodiments, the patient does not have any IV infusion associated adverse events caused by administration of the pharmaceutical composition.

In some embodiments, the patient does not have any adverse events that are muscle spasms after administration of the pharmaceutical composition. In some embodiments, the patient does not have any adverse events that are muscle spasms caused by administration of the pharmaceutical composition.

In some embodiments, the patient does not have any gastrointestinal adverse events (e.g., diarrhea or exacerbation of preexisting Inflammatory Bowel Disease (IBD)) after administration of the pharmaceutical composition. In some embodiments, the patient does not have any gastrointestinal adverse events (e.g., diarrhea or exacerbation of preexisting Inflammatory Bowel Disease (IBD)) caused by administration of the pharmaceutical composition.

In some embodiments, the antibody is administered in a pharmaceutical composition, such as those provided herein. In some embodiments, the pharmaceutical composition further comprises one or more pharmaceutically active compounds for the treatment of TAO. In some embodiments, the pharmaceutical composition further comprises corticosteroids; rituximab or other anti-CD20 antibodies; tocilizumab or other anti-IL-6 antibodies; or selenium, infliximab or other anti-TNFalpha antibodies or a thyroid-stimulating hormone receptor (TSHR) inhibitor.

Kits are also provided which are useful for carrying out embodiments described herein. The present kits comprise a first container containing or packaged in association with the above-described antibodies. The kit may also comprise another container containing or packaged in association solutions necessary or convenient for carrying out the embodiments. The containers can be made of glass, plastic or foil and can be a vial, bottle, pouch, tube, bag, etc. The kit may also contain written information, such as procedures for carrying out the embodiments or analytical information, such as the amount of reagent contained in the first container means. The kit may also contain a delivery device as well as instructions for using the delivery device. The container may be in another container apparatus, e.g., a box or a bag, along with the written information.

Yet another aspect provided for herein is a kit for detecting IGF-1R protein in a biological sample. The kit includes a container holding one or more antibodies which binds an epitope of IGF-1R protein and instructions for using the antibody for the purpose of binding to IGF-1R protein to form an immunological complex and detecting the formation of the immunological complex such that the presence or absence of the immunological complex correlates with presence or absence of IGF-1R protein in the sample. Examples of containers include multiwell plates which allow simultaneous detection of IGF-1R protein in multiple samples.

In some embodiments, antibodies that bind to an IGF-1R protein are provided. In some embodiments, the antibody is isolated. In some embodiments, the antibody binds specifically. In some embodiments, the antibody binds to an IGF-1R protein that is properly folded. In some embodiments, the antibody is specific for a specific IGF-1R conformational state (open or closed). In some embodiments, the antibody binds to an IGF-1R protein in a cell membrane. In some embodiments, the antibody binds to an IGF-1R protein that is in a cell membrane in an intact cell. In some embodiments, the antibody inhibits or neutralizes the function of an IGF-1R protein. As used herein, the term “neutralize” means that the activity or function of the protein is inhibited. The inhibition can be complete or partial. In some embodiments, the activity or function of the protein is inhibited at least 10, 20, 30, 40, 50, 60, 70, 80, 90, 95, or 99%. The percent inhibition can be based upon the function or activity of the protein in the absence of the antibody. In some embodiments, the antibody inhibits the glucose transport facilitated by IGF-1R. In some embodiments, the antibody inhibits the internalization of the IGF-1R protein.

In some embodiments, the antibody comprises a sequence as provided for herein or antigen binding fragment thereof. In some embodiments, the antibody comprises a heavy chain CDR or an antigen binding fragment thereof described herein. The heavy chain may be one or more of the heavy chains described herein. In some embodiments, the antibody comprises a light chain, or an antigen binding fragment thereof as described herein

In some embodiments, methods of detecting the presence or absence of an IGF-1R in a sample are provided, the method comprising contacting a sample with one or more antibodies described herein detecting the binding to an IGF-1R antigen by the antibody. In some embodiments, the detection of the binding indicates the presence IGF-1R antigen; or the absence of the detection of the binding to the IGF-1R antigen indicates the absence of the IGF-1R antigen. The detecting can be done with any known method, such as using a biosensor, ELISA, sandwich assay, and the like. However, in some embodiments, the method comprises detecting the presence of the protein in non-denaturing conditions. The non-denaturing conditions can be used so that the protein of interest is detected in its native, or properly folded form.

In some embodiments, methods of identifying a test antibody that binds to an epitope on IGF-1R protein, are provided, the method comprising contacting a test antibody with the epitope on IGF-1R protein and determining whether the test antibody binds to the epitope. In some embodiments, the determining comprises determining whether the test antibody binds to the protein and is competitively inhibited by an antibody comprising a sequence as provided herein. In some embodiments, the determining comprises mutating one or more residues of epitope or protein and determining binding of the test antibody to the mutated epitope, wherein if the mutation reduces binding of the test antibody as compared to the non-mutated epitope, the test antibody is deemed to bind to that epitope.

In some embodiments, methods of monitoring internalization of IGF-1R from the surface of a cell are provided. In some embodiments, the method comprising contacting the cell with an anti-IGF-1R antibody as provided herein and detecting the presence of IGF-1R in the cell or on the surface of the cell. The differences in cell surface expression can be measured and the internalization can be monitored and measured. This can be used, for example, to measure the effect of another molecule, such as a test agent, to modulate internalization of IGF-1R protein. Thus, the antibodies provided for herein can be used to identify test agents that modulate (increase or decrease) the internalization of IGF-1R protein. Test molecules that increase the internalization, which would be measured as a decrease in binding of an anti-IGF-1R antibody to IGF-1R protein on the cell surface, can be identified according to the methods provided herein. Test molecules that decrease the internalization, which would be measured as an increase in binding of an anti-IGF-1R antibody to IGF-1R protein on the cell surface, can be identified according to the methods provided herein. The surface expression can be measured by fluorescence, which can be done through a secondary antibody that recognized the IGF-1R antibodies or by labelling the anti-IGF-1R antibodies provided for herein.

In some embodiments, the subject is administered the composition at a dose of about 3 mg/kg of the antibody and has a Cmax at day 1 of about 80 μg/mL to about 95 μg/mL or about 85 μg/mL to about 95 μg/mL. In some embodiments, the Cmax at day 1 is 90 μg/mL to about 95 μg/mL. In some embodiments, the Cmax is measured or observed about, or within, 2 hours after administration of the dose to the patient. In some embodiments, the median time of Cmax occurred at about 2 hours.

In some embodiments, the subject is administered the composition at a dose of about 3 mg/kg of the antibody and has a Cmin at day 21 of about 5 μg/mL to about 10 μg/mL or about 5 μg/mL to about 6 μg/mL.

In some embodiments, the subject is administered the composition at a dose of about 10 mg/kg of the antibody and has a Cmax at day 1 of about 250 μg/mL to about 350 μg/mL, about 275 μg/mL to about 325 μg/mL, or about 285 μg/mL to about 315 μg/mL. In some embodiments, the Cmax is measured or observed about, or within, 2 hours after administration of the dose to the patient. In some embodiments, the median time of Cmax occurred at about 2 hours.

In some embodiments, the subject is administered the composition at a dose of about 10 mg/kg of the antibody and has a Cmin at day 21 of about 30 μg/mL to about 50 μg/mL, about 40 μg/mL to about 50 μg/mL, or about 45 to about 50 μg/mL.

In some embodiments, embodiments provided herein also include, but are not limited to:

### First Set of Exemplary Embodiments

1. A method of treating a patient suffering from chronic thyroid eye disease, such as moderate to severe TED, the method comprising administering a pharmaceutical composition comprising an anti-IGF-1R antibody at a dose of about 3.0 mg/kg to about 20 mg/kg, about 3.0 mg/kg, about 5.0 mg/kg, about 10 mg/kg or about 20 mg/kg comprising,
   wherein the antibody comprises a heavy chain and a light chain, wherein the heavy chain comprises a HCDR1 of SEQ ID NO: 7, a HCDR2 of SEQ ID NO: 8, and a HCDR3 of SEQ ID NO: 9 and the light chain comprises a LCDR1 of SEQ ID NO: 4, a LCDR2 of SEQ ID NO: 5 and a LCDR3 of SEQ ID NO: 6,
   wherein the patient, prior to administration of the first dose, has had symptoms of thyroid eye disease for at least or greater than one year and has one or more of the following: lid retraction greater than 2 mm, moderate or severe soft tissue involvement, exophthalmos (proptosis) of greater than or equal to 3 mm, Clinical Activity Score (CAS) from about 0 to about 7, and inconstant or constant diplopia.
2. The method of embodiment 1, wherein the exophthalmos is greater than or equal to 3 mm above the normal range for their race and gender.
3. The method of embodiments 1 or 2, wherein the patient has exophthalmos is greater than or equal to 3 mm and a CAS from about 0 to about 7 greater than 2.
4. The method of any one of embodiments 1-3, wherein the patient was diagnosed with thyroid eye disease at least one year prior to being administered the pharmaceutical composition.
5. The method of embodiment 1, wherein the pharmaceutical composition is administered by infusion, intravenously, or subcutaneously.
6. The method of embodiments 1 or 2, wherein the method comprising administering a dose to the patient about every 21 days.
7. The method of any one of embodiments 1-3, wherein the patient is administered at least 5 consecutive doses of the pharmaceutical composition, wherein the dose is administered about every 21 days.
8. The method of any one of embodiments 1-3, wherein the patient is administered at least 5 consecutive doses of the pharmaceutical composition, wherein the dose is administered about every 21 days.
9. The method of any one of embodiments 1-3, wherein the patient is administered at least 8 consecutive doses of the pharmaceutical composition, wherein the dose is administered about every 21 days.
10. The method of any one of embodiments 1-9, wherein the pharmaceutical composition comprising the antibody is administered at a dose of about 10 mg/kg.
11. The method of embodiment 10, wherein the pharmaceutical composition is administered to the patient every 21 days.
12. The method of embodiment 10, wherein the pharmaceutical composition is administered to the patient every 21 days for a total of about 5 to about 8 doses, about 5, about 6, about 7, or about 8 doses.
13. The method of any one of embodiments 1-9, wherein the pharmaceutical composition comprising the antibody is administered at a dose of about 3 mg/kg.
14. The method of embodiment 13, wherein the pharmaceutical composition is administered to the patient every 21 days.
15. The method of embodiment 13, wherein the pharmaceutical composition is administered to the patient every 21 days for a total of about 5 to about 8 doses, about 5, about 6, about 7, or about 8 doses.
16. The method of any one of embodiments 1-15, wherein the proptosis is reduced by about 1 to about 3 mm, about 1 to about 2 mm, about 2 to about 3 mm from baseline within 6 weeks of the first dose as measured by exophthalmometry or by MRI/CT.
17. The method of any one of embodiments 1-16, wherein the proptosis is reduced by about 2-3 mm from baseline within 6 weeks of the first dose as measured by exophthalmometry or by MRI/CT.
18. The method of embodiments 16 or 17, wherein the subject, prior to administration of the pharmaceutical composition, had a CAS of greater than 0, 1, 2, 3, or 4, or about 2 to about 4.
19. The method of any one of embodiments 1-18, wherein the subject is administered the composition at a dose of about 3 mg/kg and has a Cmax at day 1 of about 80 μg/mL to about 95 μg/mL or about 85 μg/mL to about 95 μg/mL.
20. The method of embodiment 19, wherein the subject is administered the composition at a dose of about 3 mg/kg and has a Cmin at day 21 of about 5 μg/mL to about 10 μg/mL or about 5 μg/mL to about 6 μg/mL.
21. The method of any one of embodiments 1-18, wherein the subject is administered the composition at a dose of about 10 mg/kg and has a Cmax at day 1 of about 250 μg/mL to about 350 μg/mL or about 275 μg/mL to about 325 μg/mL.
22. The method of embodiment 21, wherein the subject is administered the composition at a dose of about 10 mg/kg and has a Cmin at day 21 of about 30 μg/mL to about 50 μg/mL, about 40 μg/mL to about 50 μg/mL, or about 45 to about 50 μg/mL.
23. The method of any one of embodiments 1-22, wherein the patient, prior to administration of the first dose, has had symptoms of thyroid eye disease more than 2, 3, 4, 5, 6, or 7 years, or 1 to about 8 years, about 1 to about 7 years, about 1 to about 6 years, about 1 to about 5 years, about 1 to about 4 years, about 1 to about 3 years, about 1 to about 2 years, about 2 to about 8 years, about 2 to about 7 years, about 2 to about 6 years, about 2 to about 5 years, about 2 to about 4 years, about 2 to about 3 years, about 3 to about 8 years, about 3 to about 7 years, about 3 to about 5 years, about 3 to about 4 years, about 4 to about 8 years, about 4 to about 7 years, about 4 to about 6 years, about 4 to about 5 years, about 5 to about 8 years, about 5 to about 7 years, about 5 to about 6 years, about 6 to about 8 years, about 6 to about 7 years, or about 7 to about 8 years.
24. The method of any one of embodiments 1-22, wherein the patient, prior to administration of the first dose, has had symptoms of thyroid eye disease no more than 63 months, 60 months, 48 months, 36 months, 24 months, or 12 months.
25. The method of any one of embodiments 1-24, wherein the patient does not have any hearing impairment, ototoxic changes in audiometry, or hyperglycemic events after administration of the pharmaceutical composition.
26. The method of any one of embodiments 1-24, wherein the patient does not have any hearing impairment, ototoxic changes in audiometry, or hyperglycemic events caused by the administration of the pharmaceutical composition.
27. The method of any one of embodiments 1-26, wherein the light chain comprises a variable region having the amino acid sequence of SEQ ID NO: 2 and the heavy chain comprises a variable region sequence having the amino acid sequence of SEQ ID NO: 3.
28. The method of any one of embodiments 1-27, wherein the light chain comprises an amino acid sequence of SEQ ID NO: 11.
29. The method of any one of embodiments 1-28, wherein the heavy chain comprises an amino acid sequence of SEQ ID NO: 10.
30. The method of any one of embodiments 1-27, wherein the heavy chain comprises an amino acid sequence of SEQ ID NO: 10 and the light chain comprises an amino acid sequence of SEQ ID NO: 11.
31. The method of any one of embodiments 1-30, wherein after first dose of the antibody, the clinical activity score of the subject is reduced.
32. The method of any one of embodiments 1-30, wherein after two doses of the antibody, the clinical activity score of the subject is reduced.
33. The method of any one of embodiments 1-32, wherein the pharmaceutical composition comprising the antibody comprises at least one pharmaceutically acceptable excipient, wherein the pharmaceutical composition comprises the antibody at a concentration of 20 mg/mL to about 30 mg/mL.
34. The method of embodiment 33, wherein the pharmaceutical composition comprises the antibody at a concentration of about 25 mg/mL.

### Second Set of Exemplary Embodiments

1. A method of treating a patient suffering from an ocular disorder, the method comprising administering a pharmaceutical composition comprising an anti-IGF-1R antibody at a dose of about 3.0 mg/kg to about 20 mg/kg, about 3.0 mg/kg to about 10 mg/kg, about 3.0 mg/kg, about 5.0 mg/kg, about 10 mg/kg or about 20 mg/kg comprising,
   wherein the antibody comprises a heavy chain and a light chain, wherein the heavy chain comprises a HCDR1, a HCDR2, and a HCDR3 of SEQ ID NO: 10 and the light chain comprises a LCDR1, a LCDR2, and a LCDR3 of SEQ ID NO: 11,
   wherein the patient, prior to administration of the first dose of the pharmaceutical composition:
   (a) has had one or more symptoms of the ocular disorder for at least 12 months;
   (b) has had one or more symptoms of the ocular disorder for at least 15 months;
   (c) has had one or more symptoms of the ocular disorder associated with a Clinical Activity Score (CAS) of ≥4, where such symptom(s) have lessened in severity over time;
   (d) has had one or more symptoms of the ocular disorder that have reached a plateau, e.g., a static plateau;
   (e) for at least or greater than one year and has one or more of the following: lid retraction greater than 2 mm, moderate or severe soft tissue involvement, exophthalmos (proptosis) of greater than or equal to 3 mm, Clinical Activity Score (CAS) from about 0 to about 7, and inconstant or constant diplopia;
   (f) for at least or greater than 15 months and has one or more of the following: lid retraction greater than 2 mm, moderate or severe soft tissue involvement, exophthalmos (proptosis) of greater than or equal to 3 mm, Clinical Activity Score (CAS) from about 0 to about 7, and inconstant or constant diplopia; or
   (g) has had one or more of each of (a)-(f).
2. The method of embodiment 1, wherein the ocular disorder is an autoimmune associated eye disorders associated with IGF-1R signaling.
3. The method of embodiment 1, wherein the ocular disorder is an inflammatory orbital disorder associated with IGF-1R signaling.
4. The method of embodiment 1, wherein the ocular disorder is a thyroid eye disorders associated with IGF-1R signaling.
5. The method of embodiment 1, wherein the ocular disorder is thyrotoxic exophthalmos.
6. The method of embodiment 1, wherein the ocular disorder is dysthyroid ophthalmopathy.
7. The method of embodiment 1, wherein the ocular disorder is thyroid-associated ophthalmopathy (TAO), thyroid eye disease (TED), or Graves' ophthalmopathy or orbitopathy (GO).
8. The method of embodiment 7, wherein the ocular disorder is chronic TED.
9. The method of any one of the preceding embodiments, wherein the HCDR1, HCDR2, HCDR3, LCDR1, LCDR2, and LCDR3 are determined using Kabat, IMGT, Chothia or North numbering schemes.
10. A method of treating a patient suffering from chronic thyroid eye disease, such as moderate to severe TED, the method comprising administering a pharmaceutical composition comprising an anti-IGF-1R antibody at a dose of about 3.0 mg/kg to about 20 mg/kg, about 3.0 mg/kg to about 10 mg/kg, about 3.0 mg/kg, about 5.0 mg/kg, about 10 mg/kg or about 20 mg/kg comprising,
   wherein the antibody comprises a heavy chain and a light chain, wherein the heavy chain comprises a HCDR1 of SEQ ID NO: 7, a HCDR2 of SEQ ID NO: 8, and a HCDR3 of SEQ ID NO: 9 and the light chain comprises a LCDR1 of SEQ ID NO: 4, a LCDR2 of SEQ ID NO: 5 and a LCDR3 of SEQ ID NO: 6,
   wherein the patient, prior to administration of the first dose of the pharmaceutical composition, has had symptoms of thyroid eye disease for at least or greater than one year and has one or more of the following: lid retraction greater than 2 mm, moderate or severe soft tissue involvement, exophthalmos (proptosis) of greater than or equal to 3 mm, Clinical Activity Score (CAS) from about 0 to about 7, and inconstant or constant diplopia.
11. The method of any one of the preceding embodiments, wherein the exophthalmos is greater than or equal to 3 mm above the normal range for their race and gender.
12. The method of any one of the preceding embodiments, wherein the patient has exophthalmos is greater than or equal to 3 mm and a CAS from about 0 to about 7 greater than 2.
13. The method of any one of the preceding embodiments, wherein the patient was diagnosed with thyroid eye disease at least one year prior to being administered the pharmaceutical composition.
14. The method of any one of the preceding embodiments, wherein the pharmaceutical composition is administered by infusion, intravenously, or subcutaneously.
15. The method of any one of the preceding embodiments, wherein the pharmaceutical composition is administered via hypodermic needle, prefilled syringe, or autoinjection.
16. The method of any one of the preceding embodiments, wherein the method comprising administering a dose to the patient about every 21 days.
17. The method of any one of the preceding embodiments, wherein the patient is administered at least 4 or 5 consecutive doses of the pharmaceutical composition, wherein the dose is administered about every 21 days.
18. The method of any one of the preceding embodiments, wherein the patient is administered at least 6 or 7 consecutive doses of the pharmaceutical composition, wherein the dose is administered about every 21 days.
19. The method of any one of the preceding embodiments, wherein the patient is administered at least 8 consecutive doses of the pharmaceutical composition,
   wherein the dose is administered about every 21 days.
20. The method of any one of the preceding embodiments, wherein the anti-IGF-1R antibody is provided as an antigen binding fragment, form, variant, or derivative of the anti-IGF-1R antibody.
21. The method of any one of embodiments 1-20, wherein the pharmaceutical composition comprising the antibody is administered at a dose of about 3.0 mg/kg to about 10 mg/kg.
22. The method of embodiment 21, wherein the pharmaceutical composition is administered to the patient every 21 days.
23. The method of embodiment 21, wherein the pharmaceutical composition is administered to the patient every 21 days for a total of about 4 to about 8 doses, about 4, about 5, about 6, about 7, or about 8 doses.
24. The method of any one of embodiments 1-20, wherein the pharmaceutical composition comprising the antibody is administered at a dose of about 10 mg/kg.
25. The method of embodiment 24, wherein the pharmaceutical composition is administered to the patient every 21 days.
26. The method of embodiment 24, wherein the pharmaceutical composition is administered to the patient every 21 days for a total of about 4 to about 8 doses, about 4, about 5, about 6, about 7, or about 8 doses.
27. The method of any one of embodiments 1-20, wherein the pharmaceutical composition comprising the antibody is administered at a dose of about 3 mg/kg.
28. The method of embodiment 27, wherein the pharmaceutical composition is administered to the patient every 21 days.
29. The method of embodiment 27, wherein the pharmaceutical composition is administered to the patient every 21 days for a total of about 5 to about 8 doses, about 5, about 6, about 7, or about 8 doses.
30. The method of any one of the preceding embodiments, wherein the method comprises administering a subsequent dose at a dose of about 3.0 mg/kg to about 20 mg/kg, about 3.0 mg/kg to about 10 mg/kg, about 3.0 mg/kg, about 5.0 mg/kg, about 10 mg/kg or about 20 mg/kg.
31. The method of any one of the preceding embodiments, wherein the proptosis is reduced by about 1 to about 3 mm, about 1 to about 2 mm, about 2 to about 3 mm from baseline within 6 weeks of the first dose as measured by exophthalmometry or by MRI/CT.
32. The method of any one of the preceding embodiments, wherein the proptosis is reduced by about 2-3 mm from baseline within 6 weeks of the first dose as measured by exophthalmometry or by MRI/CT.
33. The method of embodiments 31 or 32, wherein the subject, prior to administration of the pharmaceutical composition, had a CAS of greater than 0, 1, 2, 3, or 4, or about 2 to about 4.
34. The method of any one of the preceding embodiments, wherein the subject is administered the composition at a dose of about 3 mg/kg and has a Cmax at day 1 of about 80 μg/mL to about 95 μg/mL or about 85 μg/mL to about 95 μg/mL.
35. The method of embodiment 34, wherein the subject is administered the composition at a dose of about 3 mg/kg and has a Cmin at day 21 of about 5 μg/mL to about 10 μg/mL or about 5 μg/mL to about 6 μg/mL.
36. The method of any one of the preceding embodiments, wherein the subject is administered the composition at a dose of about 10 mg/kg and has a Cmax at day 1 of about 250 μg/mL to about 350 μg/mL or about 275 μg/mL to about 325 μg/mL.
37. The method of embodiment 36, wherein the subject is administered the composition at a dose of about 10 mg/kg and has a Cmin at day 21 of about 30 μg/mL to about 50 μg/mL, about 40 μg/mL to about 50 μg/mL, or about 45 to about 50 μg/mL.
38. The method of any one of the preceding embodiments, wherein the patient, prior to administration of the first dose of the pharmaceutical composition, has had symptoms of ocular disorder or thyroid eye disease more than 2, 3, 4, 5, 6, or 7 years, or 1 to about 8 years, about 1 to about 7 years, about 1 to about 6 years, about 1 to about 5 years, about 1 to about 4 years, about 1 to about 3 years, about 1 to about 2 years, about 2 to about 8 years, about 2 to about 7 years, about 2 to about 6 years, about 2 to about 5 years, about 2 to about 4 years, about 2 to about 3 years, about 3 to about 8 years, about 3 to about 7 years, about 3 to about 5 years, about 3 to about 4 years, about 4 to about 8 years, about 4 to about 7 years, about 4 to about 6 years, about 4 to about 5 years, about 5 to about 8 years, about 5 to about 7 years, about 5 to about 6 years, about 6 to about 8 years, about 6 to about 7 years, or about 7 to about 8 years.
39. The method of any one of the preceding embodiments, wherein the patient, prior to administration of the first dose, has had symptoms of thyroid eye disease no more than 63 months, 60 months, 48 months, 36 months, 24 months, or 12 months.
40. The method of any one of the preceding embodiments, wherein the patient does not have any hearing impairment, ototoxic changes in audiometry, or hyperglycemic events after administration of the pharmaceutical composition.
41. The method of any one of the preceding embodiments, wherein the patient does not have any hearing impairment, ototoxic changes in audiometry, or hyperglycemic events caused by the administration of the pharmaceutical composition.
42. The method of any one of the preceding embodiments, wherein after first dose of the antibody, the clinical activity score of the subject is reduced.
43. The method of any one of the preceding embodiments, wherein after two doses of the antibody, the clinical activity score of the subject is reduced.
44. The method of any one of the preceding embodiments, wherein the pharmaceutical composition comprising the antibody comprises at least one pharmaceutically acceptable excipient, wherein the pharmaceutical composition comprises the antibody at a concentration of 20 mg/mL to about 30 mg/mL.
45. The method of embodiment 33, wherein the pharmaceutical composition comprises the antibody at a concentration of about 25 mg/mL.
46. A method of treating a patient suffering from chronic thyroid eye disease (TED), the method comprising administering a pharmaceutical composition comprising an anti-IGF-1R antibody at a therapeutically effective dosing regimen,
   wherein the antibody comprises a heavy chain and a light chain, wherein the heavy chain comprises a HCDR1, a HCDR2, and a HCDR3 of SEQ ID NO: 10 and the light chain comprises a LCDR1, a LCDR2, and a LCDR3 of SEQ ID NO: 11.
47. A method of treating a patient suffering from inactive thyroid eye disease (TED), the method comprising administering a pharmaceutical composition comprising an anti-IGF-1R antibody at a therapeutically effective dosing regimen,
   wherein the antibody comprises a heavy chain and a light chain, wherein the heavy chain comprises a HCDR1, a HCDR2, and a HCDR3 of SEQ ID NO: 10 and the light chain comprises a LCDR1 of, a LCDR2, and a LCDR3 of SEQ ID NO: 11.
48. A method of treating a patient suffering from thyroid eye disease (TED), the method comprising administering a pharmaceutical composition comprising an anti-IGF-1R antibody at a therapeutically effective dosing regimen,
   wherein the antibody comprises a heavy chain and a light chain, wherein the heavy chain comprises a HCDR1, a HCDR2, and a HCDR3 of SEQ ID NO: 10 and the light chain comprises a LCDR1 of, a LCDR2, and a LCDR3 of SEQ ID NO: 11, wherein the patient, prior to administration of the first dose of the pharmaceutical composition has had one or more symptoms of the ocular disorder associated with a Clinical Activity Score (CAS) of ≥4, where such symptom(s) have lessened in severity over time
49. A method of treating a patient suffering from thyroid eye disease (TED), the method comprising administering a pharmaceutical composition comprising an anti-IGF-1R antibody at a therapeutically effective dosing regimen,
   wherein the antibody comprises a heavy chain and a light chain, wherein the heavy chain comprises a HCDR1, a HCDR2, and a HCDR3 of SEQ ID NO: 10 and the light chain comprises a LCDR1 of, a LCDR2, and a LCDR3 of SEQ ID NO: 11, wherein the patient, prior to administration of the first dose of the pharmaceutical composition has had one or more symptoms of the ocular disorder that have reached a plateau, e.g., a static plateau.
50. A method of treating a patient suffering from thyroid eye disease (TED), the method comprising administering a pharmaceutical composition comprising an anti-IGF-1R antibody at a therapeutically effective dosing regimen,
   wherein the antibody comprises a heavy chain and a light chain, wherein the heavy chain comprises a HCDR1, a HCDR2, and a HCDR3 of SEQ ID NO: 10 and the light chain comprises a LCDR1, a LCDR2, and a LCDR3 of SEQ ID NO: 11,
   wherein the patient, prior to treatment, has had one or more symptoms of thyroid eye disease for at least 12 months.
51. The method of any one of embodiments 46-49, wherein the patient, prior to treatment, has had one or more symptoms of thyroid eye disease for at least 12 months.
52. The method of any one of embodiments 46-51, wherein the patient suffers one or more symptoms of TED selected from the group consisting of lid retraction greater than 2 mm, exophthalmos (proptosis) of greater than or equal to 3 mm, Clinical Activity Score (CAS) from about 0 to about 7, and inconstant or constant diplopia.
53. The method of embodiment 52, wherein the exophthalmos is greater than or equal to 3 mm above the normal range for their race and gender.
54. The method of any one of embodiments 46-53, wherein the patient, prior to treatment, had a CAS of or greater than 0, 1, 2, 3, or 4.
55. The method of any one of embodiments 46-54, wherein the patient, prior to treatment, had a CAS of greater than 2.
56 The method of any one of embodiments 46-55, wherein the patient, prior to treatment, had a CAS less than 2.
57. The method of any one of embodiments 46-55, wherein the patient, prior to treatment, had a CAS of 2-4.
58. The method of any one of embodiments 46-57, wherein the patient, prior to treatment, has had one or more symptoms of thyroid eye disease for at least 15 months.
59. A method of treating a patient suffering from thyroid eye disease (TED), the method comprising administering a pharmaceutical composition comprising an anti-IGF-1R antibody at a therapeutically effective dosing regimen, wherein the antibody comprises a heavy chain and a light chain, wherein the heavy chain comprises a HCDR1, a HCDR2, and a HCDR3 of SEQ ID NO: 10 and the light chain comprises a LCDR1, a LCDR2, and a LCDR3 of SEQ ID NO: 11, wherein the patient, prior to treatment, had a Clinical Activity Score (CAS) of 2 or less.
60. The method of any one of embodiments 46-59, wherein the patient exhibits fibrosis.
61. A method of treating fibrosis associated with thyroid eye disease (TED), the method comprising administering to a patient in need of treatment an anti-IGF-1R antibody at a therapeutically effective dosing regimen, wherein the antibody comprises a heavy chain and a light chain, wherein the heavy chain comprises a HCDR1, a HCDR2, and a HCDR3 of SEQ ID NO: 10 and the light chain comprises a LCDR1, a LCDR2, and a LCDR3 of SEQ ID NO: 116.
62. The method of any one of embodiments 59-61, wherein the patient, prior to treatment, had a Clinical Activity Score (CAS) of 0 or 1.
63. The method of any one of embodiments 59-62, wherein the patient has one or more symptoms of TED selected from the group consisting of lid retraction greater than 2 mm, exophthalmos (proptosis) of greater than or equal to 3 mm above the normal range for their race and gender, and inconstant or constant diplopia.
64. The method of any one of embodiments 46-63, wherein the patient, prior to treatment, has had one or more symptoms of thyroid eye disease for at least 2 years.
65. The method of any one of embodiments 46-64, wherein the therapeutically effective dosing regimen comprises administering to the patient a first dose of 3.0 mg/kg to 20 mg/kg.
66. The method of embodiment 65, wherein the first dose is 3.0 mg/kg, 5.0 mg/kg, 10 mg/kg, or 20 mg/kg.
67. The method of any one of embodiments 46-66, wherein the therapeutically effective dosing regimen comprises administering to the patient a subsequent dose.
68. The method of embodiment 67, wherein the subsequent dose is 3.0 mg/kg to 20 mg/kg.
69. The method of embodiment 68, wherein the subsequent dose is 3.0 mg/kg, 5.0 mg/kg, 10 mg/kg, or 20 mg/kg.
70. The method of any one of embodiments 68-69, wherein the subsequent dose is administered to the patient once every two weeks, once every three weeks, once every four weeks, once every month, once every five weeks, or once every six weeks.
71. The method of embodiment 70, wherein the subsequent dose is administered to the patient once every three weeks.
72. The method of any one of embodiments 67-71, wherein the patient is administered at least 3, 4, 5, 6, 7, 8, 9 or 10 subsequent doses.
73. The method of embodiment 72, wherein the patient is administered at least 4 subsequent doses.
74. The method of embodiment 72, wherein the patient is administered at least 5 subsequent doses.
75. The method of embodiment 72, wherein the patient is administered at least 8 subsequent doses.
76. The method of any one of embodiments 46-75, wherein the administration of the therapeutically effective dosing regimen results in reduction of the proptosis by about 1 to about 3 mm, about 1 to about 2 mm, about 2 to about 3 mm from baseline within 6 weeks of the first dose as measured by exophthalmometry or by MRI/CT.
77. The method of embodiment 76, wherein the proptosis is reduced by about 2-3 mm from baseline within 6 weeks of the first dose as measured by exophthalmometry or by MRI/CT.
78. The method of any one of embodiments 46-77, wherein the patient does not have hearing impairment, ototoxic changes in audiometry, or hyperglycemic levels in the patient during the treatment.
79. The method of any one of embodiments 46-78, wherein the light chain comprises a variable region having the amino acid sequence of SEQ ID NO: 2 and the heavy chain comprises a variable region sequence having the amino acid sequence of SEQ ID NO: 3.
80. The method of any one of the preceding embodiments, wherein the light chain comprises an amino acid sequence of SEQ ID NO: 11.
81. The method of any one of embodiments, wherein the heavy chain comprises an amino acid sequence of SEQ ID NO: 10.
82. The method of any one of embodiments, wherein the heavy chain comprises an amino acid sequence of SEQ ID NO: 10 and the light chain comprises an amino acid sequence of SEQ ID NO: 11.
83. The method of any one of the preceding embodiments, wherein the heavy chain comprises a HCDR1 of SEQ ID NO: 7, a HCDR2 of SEQ ID NO: 8, and a HCDR3 of SEQ ID NO: 9, and the light chain comprises a LCDR1 of SEQ ID NO: 4, a LCDR2 of SEQ ID NO: 5 and a LCDR3 of SEQ ID NO: 6.
84. The method of any one of embodiments 1-82, wherein the heavy chain comprises a HCDR1 of SEQ ID NO: 29, a HCDR2 of SEQ ID NO: 30, and a HCDR3 of SEQ ID NO: 31 and the light chain comprises a LCDR1 of SEQ ID NO: 27, a LCDR2 of SEQ ID NO: 28 and a LCDR3 of SEQ ID NO: 6.
85. The method of any one of embodiments 1-82, wherein the heavy chain comprises a HCDR1 of SEQ ID NO: 32, a HCDR2 of SEQ ID NO: 33, and a HCDR3 of SEQ ID NO: 9 and the light chain comprises a LCDR1 of SEQ ID NO: 4, a LCDR2 of SEQ ID NO: 5 and a LCDR3 of SEQ ID NO: 6.
86. The method of any one of embodiments 1-82, wherein the heavy chain comprises a HCDR1 of SEQ ID NO: 35, a HCDR2 of SEQ ID NO: 36, and a HCDR3 of SEQ ID NO: 31 and the light chain comprises a LCDR1 of SEQ ID NO: 4, a LCDR2 of SEQ ID NO: 34 and a LCDR3 of SEQ ID NO: 6.
87. The method of any one of the preceding embodiments, wherein the patient achieves an improvement in one or more parameters selected from: proptosis, CAS, extraocular muscle volume, orbital fat volume, manual measurement of lid retraction, Graves' Orbitopathy-Quality of Life (GO-QoL) combined score, GO-QoL activity subscale, GO-QoL appearance subscale, visual acuity, Gorman Subjective Diplopia Score, and EQ-5D-5L QoL questionnaire.
88. The method of embodiment 87, wherein the patient achieves an improvement in at least two or more parameters.
89. The method of embodiment 87, wherein the patient achieves an improvement in at least three or more parameters.
90. The method of embodiment 87, wherein the patient achieves an improvement in at least four or more parameters.

The subject matter is now described with reference to the following examples. These examples are provided for the purpose of illustration only and the claims should in no way be construed as being limited to these examples, but rather should be construed to encompass any and all variations which become evident as a result of the teaching provided herein.

Those of skill in the art will readily recognize a variety of non-critical parameters that could be changed or modified to yield essentially similar results.

### EXAMPLES

### Example 1. VRDN-5000 Effectively Treats Patients with Chronic Thyroid Eye Disease

Patients with chronic thyroid eye disease (proptosis of ≥3 mm above normal for race and gender; Clinical Activity Score (CAS) 0-7; with the onset of thyroid eye disease symptoms greater than or equal to 12 months prior to treatment) were randomized in a double-blind trial, placebo controlled clinical trial and administered the antibody at a dose of 3 mg/kg every three weeks for 2 doses (n=7); at a dose of 10 mg/kg every three weeks for 2 doses (n=7), or placebo (n=2 for 10 mg/kg cohort; n=3 for 3 mg/kg cohort). The following table illustrates the baseline patient characteristics for those who received 2 infusions.

TABLE 9
Baseline Patient Characteristics For Those Who Received 2 Infusions

| | VRDN-5000 | VRDN-5000 | VRDN-5000 | |
|---|---|---|---|---|
| | (3 or 10 mg/kg) | 3 mg/kg | 10 mg/kg | Placebo |
| n (number of | 12 | 6 | 6 | 5 |
| patients) | | | | |
| Proptosis by | 22.2 (1.2) | 23.4 (1.1) | 21.1 (2.2) | 25.0 (1.6) |
| exophthalmometry, | | | | |
| mean (SEM) | | | | |
| CAS, mean (SEM) | 3.3 (0.8) | 4.0 (1.0) | 2.5 (1.1) | 2.8 (0.8) |
| Diplopia, n (%) | 5 (41.7%) | 2 (33.3%) | 3 (50.0%) | 2 (40.0%) |
| Gorman, mean | 0.9 (0.4) | 1.0 (0.6) | 0.8 (0.4) | 0.6 (0.4) |
| (SEM) | | | | |
| Months since onset | 94.0 (33.7); | 92.0 (51.7); | 96.0 (48.2); | 194.4 (81.2); |
| of TED | (12, 348) | (24, 348) | (12, 312) | (24, 432) |
| signs/symptoms, | | | | |
| mean (SEM); | | | | |
| (Minimum, | | | | |
| Maximum) | | | | |
| Age, years (SEM) | 50.7 (3.3) | 53.0 (3.7) | 48.3 (5.6) | 49.4 (4.4) |
| Female, n (%) | 10 (83.3%) | 6 (100.0%) | 4 (66.7%) | 5 (100.0%) |

The following represents the available data from the study as of May 30, 2023, and is preliminary.

After administration no safety signals were observed, such as no hearing impairment adverse events, no ototoxic changes in audiometry, or no hyperglycemic events.

In the 10 mg/kg dose cohort after only 2 doses showed a 50% proptosis responder rate at week 6, an average of −1.8 mm change from baseline in proptosis at week 6, as measured by exophthalmometry, an average of −1.5 mm change from baseline in proptosis at week 6, as measured by MRI/CT, and a −2.8 reduction in CAS score for those who were entered the study with a CAS score greater than zero (0).

In the 3 mg/kg dose after only 2 doses showed a 33% proptosis responder rate at week 6, an average of −1.5 mm change from baseline in proptosis at week 6, as measured by exophthalmometry, an average of −2.6 mm change from baseline in proptosis at week 6, as measured by MRI/CT; a −2.0 reduction in CAS score for those who were entered the study with a CAS score greater than zero (0).

Across both 3 mg/kg and 10 mg/kg cohorts there was an average reduction of 2.0 mm change from baseline in proptosis at week 6 (n=8) vs −0.2 mm placebo response (n=5), as measured by MRI/CT.

For patients with a baseline proptosis >24 mm a higher week 6 proptosis change from baseline was observed.

However, in the 10 mg/kg cohort the proptosis response was observed despite the relative floor effect of low baseline proptosis.

A reduction in proptosis was observed in patients with a CAS score of 0 or 1 as compared to placebo. The data is summarized in the table below.

TABLE 10

| | | | Base Mean | Week 6 | Change |
|---|---|---|---|---|---|
| | | N | (SD) | Mean (SD) | Mean (SD) |
| Chronic | VRDN-5000 | 3 | 20.2 (6.3) | 18.8 (6.4) | −1.3 mm (0.8) |
| TED | Placebo | 1 | 24.0 (NA) | 24.0 (NA) | 0 mm (NA) |
| 10 mg/kg | | | | | |
| Dose | | | | | |
| Chronic | VRDN-5000 | 2 | 23.8 (4.6) | 22 (2.8) | −1.8 mm (1.8) |
| TED | Placebo | 1 | 26.0 (NA) | 25.0 (NA) | −1.0 mm (NA) |
| 3 mg/kg dose | | | | | |
| Overall | VRDN-5000 | 5 | 21.6 (5.4) | 20.1 (5.0) | −1.5 mm (1.1) |
| (combined | Placebo | 2 | 25.0 (1.4) | 24.5 (0.7) | −0.5 mm (0.7) |
| patient | | | | | |
| cohorts) | | | | | |

The data in patients with chronic thyroid eye disease is summarized in the table as shown in FIG. 15.

These results observed within 6 weeks of treating chronic thyroid eye disease patients were surprising and could not have been predicted.

### Example 2. VRDN-5000 in Active and Chronic TED Patients Treated with Doses of 3 mg/Kg and 10 mg/Kg have Consistent PK Levels Across Cohorts

Patients were treated with either 3 mg/kg or 10 mg/kg of VRDN-5000 and the Cmax and Cmin were measured in patients with active or chronic thyroid eye disease and it was found that the Cmax and Cmin were similar between both cohorts. The median time of Cmax occurred at about 2 hours post-infusion of the pharmaceutical composition comprising VRDN-5000. The data is illustrated in the following table.

TABLE 11

| | Dose | Cₘₐₓ Day 1 | Cₘᵢₙ Day 21 |
|---|---|---|---|
| Cohort | (mg/kg) | (μg/mL) | (μg/mL) |
| Active TED | 3 | 88.9 (17.0, 9) | 7.95 (3.18, 9) |
| Chronic TED | 3 | 89.7 (17.7, 6) | 5.78 (NA, 1) |
| Active TED | 10 | 264 (30.2, 6) | 31.0 (6.95, 5) |
| Chronic TED | 10 | 303 (68.4, 6) | 43.4 (7.78, 5) |

As the data demonstrates, the chronic subjects on average, had a reduction or improvement in proptosis or other symptoms of thyroid eye disease as measured by CAS score, which was not observed in the placebo cohort. These data demonstrate the unexpected results of how quickly VRDN-5000 can achieve a therapeutic effect, even in chronic TED patients, which could not have been predicted.

### Example 3. VRDN-5000 Effectively Treats Patients with Thyroid Eye Disease Symptoms that Began More than 15 Months Ago

This example describes a Phase 3, randomized, double-masked, placebo-controlled study to examine the safety, tolerability, and efficacy of VRDN-5000 in participants with chronic TED. This study expands on the earlier trial in participants with chronic TED which demonstrated rapid, marked improvements in proptosis and CAS at Week 6 following two infusions of VRDN-5000 at doses of either 3 mg/kg or 10 mg/kg.

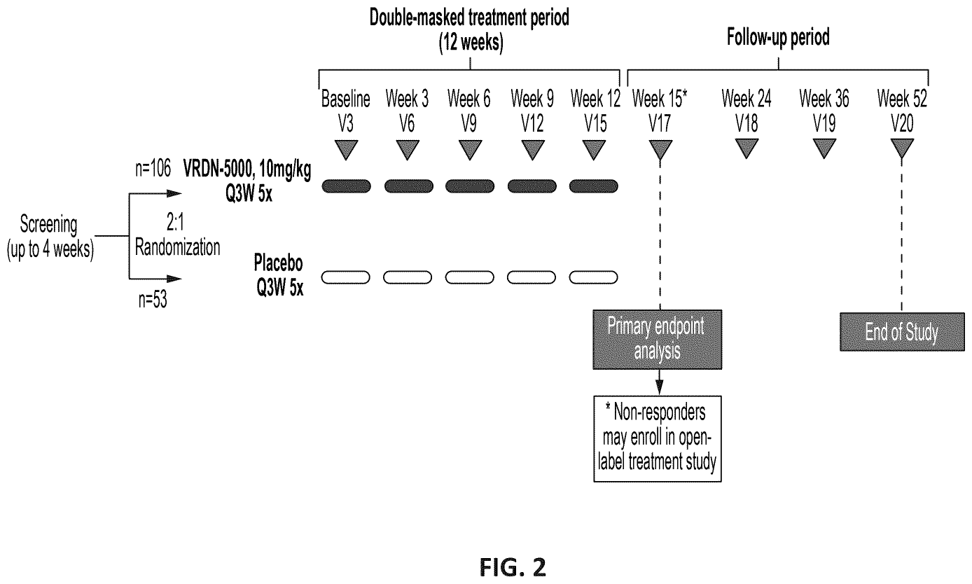

This study will first consist of a screening period followed by a double-masked treatment period lasting 12 weeks, and double masked follow-up period lasting 40 weeks (FIG. 2). Participants with chronic TED will be randomized 2:1 into a treatment arm and a placebo arm to receive 5 intravenous infusions of 10 mg/kg VRDN-5000 or placebo respectively every three weeks. A participant who does not achieve a >2 mm reduction from baseline in proptosis (as measured by MRI/CT) in the most proptotic eye at 3 weeks post the fifth IV infusion (i.e., Week 15) will be defined as a non-responder. Participants who are non-responders at Week 15 may enroll in an open-label treatment study where they will receive 5 further IV infusions of VRDN-5000 at 3-week intervals for a further 12 weeks.

### Inclusion Criteria

All participants in the study must satisfy all of the following criteria:
1. Be able to understand the study procedures and the risks involved and be willing to provide written informed consent before the first study-related activity
2. Be an adult male or female participant, at least 18 years of age or older and under the age of 75
3. Have had a clinical diagnosis of TED, with any CAS (0-7)

4. Have moderate to severe (i.e., has an appreciable impact on daily living) chronic TED associated with proptosis of ≥3 mm as measured by ophthalmometer above normal values for race and gender in the opinion of the investigator, and ≥17 mm at pre-dose baseline (Day −1 or Day 1) in the study eye as measured by exophthalmometer
5. Have documented evidence of ocular symptoms or signs associated with chronic TED that began >15 months prior to study screening
6. VRDN-5000 can be started concomitantly with attempts to achieve euthyroid status. Underlying thyroid status is not an inclusion criterion.
7. Not require immediate ophthalmological or orbital surgery in the study eye for any reason
8. VRDN-5000 can be used with caution in participants with diabetes mellitus. Diabetic participants should be monitored by their general practitioners or other appropriately trained personnel and have at study entry a glycated hemoglobin (HbA1c) of <8.5%
9. If female, have a negative serum pregnancy test at screening and further negative urine pregnancy tests immediately before each dose of study medication.
   If the participant is a woman of childbearing potential (including those with <2 years since the onset of menopause or not surgically sterile by hysterectomy, bilateral salpingectomy or bilateral oophorectomy); such participants must agree to use an acceptable method of contraception such as a condom and a second highly effective method of contraception from screening up to and including 100 days after the last dose of study medication. If the participant is initiating hormonal contraception at time of screening or within one cycle of Day 1, participant agrees to use a double-barrier method of contraception until completing one-full cycle of hormonal contraception. An acceptable double-barrier combination method is a condom with either diaphragm or sponge with spermicide
10. Be surgically sterile males for at least 6 weeks, or agree to use an acceptable method of contraception such as a condom and a second highly effective method of contraception from screening up to and including 100 days after the last dose of study medication
11. Be willing and able to comply with all the requirements of the protocol for the entire duration of the study

### Exclusion Criteria

All participants in the study must not satisfy any of the following criteria:
1. Have received prior treatment with another anti-IGF-1R therapy
2. Have received systemic corticosteroids for any condition, including TED, or selenium within 2 weeks prior to the first dose of study medication (topical steroids including eye drops or multivitamins that contain selenium are permitted). Also exclusionary is periocular (including intraorbital) or intraocular administration of corticosteroids within 3 months prior to the first dose of study medication or having received greater than 3 periocular or intraocular corticosteroid injections at any time
3. Have received other immunosuppressive agents, including rituximab, tocilizumab, secukimumab, satralizumab or anti-FcRn's for any condition (including TED) within 8 weeks prior to the first dose of study medication or have received intraorbital administration of other such immunosuppressive agents at any time
4. Have received any other therapy for TED within 8 weeks prior to the first dose of study medication (artificial tears are permitted)
5. Have received an investigational agent for any condition within 8 weeks prior to the first dose of study medication
6. Have a compressive optic neuropathy of TED that is expected to require surgical decompression in the immediate future
7. Have corneal decompensation in the study eye unresponsive to medical management
8. Have a decrease in CAS of ≥2 points in the study eye between screening assessment and Day −1 for participants with a screening CAS >2
9. Have a decrease in proptosis of ≥2 mm as measured by exophthalmometer in either eye between screening assessment and Day −1
10. Have had previous orbital irradiation for TED to the study eye's orbit or decompression surgery involving excision of fat for TED to the study eye's orbit
11. Have a pre-existing ophthalmic condition in the study eye which in the opinion of the Investigator, would confound interpretation of the study results
12. Have history of or screening audiometry assessment of significant (as determined by the Investigator) ear pathology, relevant ear surgery or hearing loss
13. Have inflammatory bowel disease (e.g., biopsy proven or clinical evidence of inflammatory bowel disease)
14. Be a pregnant or lactating woman
15. Be an active alcoholic or illicit drug user or considered at high risk of relapse by the Investigator
16. Have a known hypersensitivity to any of the components of VRDN-5000 or placebo formulations, or prior hypersensitivity to monoclonal antibodies (mAbs)
17. Have any condition, which in the opinion of the Investigator, would preclude inclusion in the study
18. Have a positive test for human immunodeficiency virus (HIV-1 and HIV-2)
19. Have a positive test for active hepatitis B or hepatitis C infection
20. Have previously participated in this study or any study of VRDN-5000
21. Have received radioactive iodine (RAI) treatment for any condition within 8 weeks prior to the first dose of study medication

Prior thyroidectomy, radioactive iodine (RAI) treatment, or orbital decompression surgery limited to bone only are not exclusions. (If fat excision occurred with orbital decompression, participant is excluded, as noted in exclusions above.)

### Efficacy Endpoints

The primary efficacy endpoint to be assessed is the Proptosis Responder Rate and the Clinical Activity Responder Rate in the most proptotic eye. The Proptosis Responder Rate will be assessed as a reduction of proptosis of ≥2 mm from baseline (without a corresponding increase of ≥2 mm in the other eye) at 3 weeks post the fifth IV infusion (i.e., Week 15). The Clinical Activity Responder Rate will be assessed as no worsening in CAS from baseline (without a corresponding increase of ≥2 points in the other eye) at 3 weeks post the fifth IV infusion (i.e., Week 15).

Key secondary endpoints will include:
Change from baseline in proptosis for the most proptotic eye at Week 15
Proptosis Responder Rate in the most proptotic eye as measured by exophthalmometer at Week 15
Change from baseline in proptosis in the most proptotic eye as measured by exophthalmometer at Week 15
Clinical Activity Responder Rate in the most proptotic eye at Week 15
Overall Responder Rate in the most proptotic eye at Week 15
Diplopia Resolution Rate (i.e., reduction in Gorman Subjective Diplopia Score to 0 from baseline for participants with baseline Diplopia Score >0) at Week 15

Exploratory efficacy endpoints will further include:
Proptosis Responder Rate in the most proptotic eye at Week 24 (12 weeks post fifth IV infusion), Week 36 (24 weeks post fifth IV infusion) and Week 52
Proptosis Responder Rate in the other eye (i.e., reduction of proptosis of ≥2 mm from baseline) at Weeks 15, 24, 36 and 52
Durability of Proptosis Response in the most proptotic eye at Weeks 24, 36 and 52
Time to First Proptosis Response in the most proptotic eye
Clinical Activity Responder Rate in the most proptotic eye at Weeks 24, 36 and 52
Clinical Activity Responder Rate in the other eye at Weeks 15, 24, 36 and 52
Overall Responder Rate in the most proptotic eye at Weeks 24, 36 and 52
Overall Responder Rate in the other eye at Weeks 15, 24, 36 and 52
Diplopia Resolution Rate at Weeks 24, 36 and 52
Change from baseline in the most proptotic eye and other eye where applicable in the following parameters at Weeks 15, 24, 36 and 52:
   Proptosis
   CAS
   Extraocular muscle volume
   Orbital fat volume
   Manual measurement of lid retraction
   Graves' Orbitopathy-Quality of Life (GO-QoL) combined score
   GO-QoL activity subscale
   GO-QoL appearance subscale
   Visual Acuity (VA)
   Gorman Subjective Diplopia Score
EQ-5D-5L QoL questionnaire
VRDN-5000, IGF-1 and anti-drug antibodies (ADA) at various time points pre- and post-IV infusions.

### Safety Endpoints

Adverse Events (AEs) and Serious Adverse Events (SAEs) will be monitored and recorded throughout the duration of the study. All clinically significant changes in other safety measurements will be recorded as AEs.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present embodiments are not to be limited in scope by the specific embodiments described herein. Indeed, various modifications in addition to those described herein will become apparent to those skilled in the art from the foregoing description and the accompanying figures. Such modifications are intended to fall within the scope of the embodiments and any appended claims.

The present specification is considered to be sufficient to enable one skilled in the art to practice the embodiments. Various modifications in addition to those shown and described herein will become apparent to those skilled in the art from the foregoing description and fall within the scope of the present disclosure and any appended claims.

1. A method of treating a patient suffering from chronic thyroid eye disease (TED), the method comprising administering a pharmaceutical composition comprising an anti-IGF-1R antibody at a therapeutically effective dosing regimen,
wherein the antibody comprises a heavy chain and a light chain, wherein the heavy chain comprises a HCDR1 of SEQ ID NO: 7, a HCDR2 of SEQ ID NO: 8, and a HCDR3 of SEQ ID NO: 9 and the light chain comprises a LCDR1 of SEQ ID NO: 4, a LCDR2 of SEQ ID NO: 5 and a LCDR3 of SEQ ID NO: 6.

2. A method of treating a patient suffering from thyroid eye disease (TED), the method comprising administering a pharmaceutical composition comprising an anti-IGF-1R antibody at a therapeutically effective dosing regimen,
wherein the antibody comprises a heavy chain and a light chain, wherein the heavy chain comprises a HCDR1 of SEQ ID NO: 7, a HCDR2 of SEQ ID NO: 8, and a HCDR3 of SEQ ID NO: 9 and the light chain comprises a LCDR1 of SEQ ID NO: 4, a LCDR2 of SEQ ID NO: 5 and a LCDR3 of SEQ ID NO: 6,
wherein the patient, prior to treatment, has had one or more symptoms of thyroid eye disease for at least 12 months.

3. The method of claim 1, wherein the patient, prior to treatment, has had one or more symptoms of thyroid eye disease for at least 12 months.

4. The method of any one of the preceding claims, wherein the patient suffers one or more symptoms of TED selected from the group consisting of lid retraction greater than 2 mm, exophthalmos (proptosis) of greater than or equal to 3 mm, Clinical Activity Score (CAS) from about 0 to about 7, and inconstant or constant diplopia.

5. The method of claim 4, wherein the exophthalmos is greater than or equal to 3 mm above the normal range for their race and gender.

6. The method of any one of the preceding claims, wherein the patient, prior to treatment, had a CAS of or greater than 0, 1, 2, 3, or 4.

7. The method of any one of the preceding claims, wherein the patient, prior to treatment, had a CAS of greater than 2.

8. The method of any one of claims 1-6, wherein the patient, prior to treatment, had a CAS less than 2.

9. The method of any one of claims 1-7, wherein the patient, prior to treatment, had a CAS of 2-4.

10. The method of any one of the preceding claims, wherein the patient, prior to treatment, has had one or more symptoms of thyroid eye disease for at least 15 months.

11. A method of treating a patient suffering from inactive thyroid eye disease (TED), the method comprising administering a pharmaceutical composition comprising an anti-IGF-1R antibody at a therapeutically effective dosing regimen,
wherein the antibody comprises a heavy chain and a light chain, wherein the heavy chain comprises a HCDR1 of SEQ ID NO: 7, a HCDR2 of SEQ ID NO: 8, and a HCDR3 of SEQ ID NO: 9 and the light chain comprises a LCDR1 of SEQ ID NO: 4, a LCDR2 of SEQ ID NO: 5 and a LCDR3 of SEQ ID NO: 6.

12. A method of treating a patient suffering from thyroid eye disease (TED), the method comprising administering a pharmaceutical composition comprising an anti-IGF-1R antibody at a therapeutically effective dosing regimen,
wherein the antibody comprises a heavy chain and a light chain, wherein the heavy chain comprises a HCDR1 of SEQ ID NO: 7, a HCDR2 of SEQ ID NO: 8, and a HCDR3 of SEQ ID NO: 9 and the light chain comprises a LCDR1 of SEQ ID NO: 4, a LCDR2 of SEQ ID NO: 5 and a LCDR3 of SEQ ID NO: 6,
wherein the patient, prior to treatment, had a Clinical Activity Score (CAS) of 2 or less.

13. The method of any one of the preceding claims, wherein the patient exhibits fibrosis.

14. A method of treating fibrosis associated with thyroid eye disease (TED), the method comprising administering to a patient in need of treatment an anti-IGF-1R antibody at a therapeutically effective dosing regimen,
wherein the antibody comprises a heavy chain and a light chain, wherein the heavy chain comprises a HCDR1 of SEQ ID NO: 7, a HCDR2 of SEQ ID NO: 8, and a HCDR3 of SEQ ID NO: 9 and the light chain comprises a LCDR1 of SEQ ID NO: 4, a LCDR2 of SEQ ID NO: 5 and a LCDR3 of SEQ ID NO: 6.

15. The method of any one of claims 11-14, wherein the patient, prior to treatment, had a Clinical Activity Score (CAS) of 0 or 1.

16. The method of any one of claims 11-15, wherein the patient has one or more symptoms of TED selected from the group consisting of lid retraction greater than 2 mm, exophthalmos (proptosis) of greater than or equal to 3 mm above the normal range for their race and gender, and inconstant or constant diplopia.

17. The method of any one of the preceding claims, wherein the patient, prior to treatment, has had one or more symptoms of thyroid eye disease for at least 2 years.

18. The method of any one of the preceding claims, wherein the therapeutically effective dosing regimen comprises administering to the patient a first dose of 3.0 mg/kg to 20 mg/kg.

19. The method of claim 18, wherein the first dose is 3.0 mg/kg, 5.0 mg/kg, 10 mg/kg, or 20 mg/kg.

20. The method of any one of the preceding claims, wherein the therapeutically effective dosing regimen comprises administering to the patient a subsequent dose.

21. The method of claim 20, wherein the subsequent dose is 3.0 mg/kg to 20 mg/kg.

22. The method of claim 21, wherein the subsequent dose is 3.0 mg/kg, 5.0 mg/kg, 10 mg/kg, or 20 mg/kg.

23. The method of any one of claims 20-22, wherein the subsequent dose is administered to the patient once every two weeks, once every three weeks, once every four weeks, once every month, once every five weeks, or once every six weeks.

24. The method of claim 23, wherein the subsequent dose is administered to the patient once every three weeks.

25. The method of any one of claims 20-24, wherein the patient is administered at least 3, 4, 5, 6, 7, 8, 9 or 10 subsequent doses.

26. The method of claim 25, wherein the patient is administered at least 5 subsequent doses.

27. The method of claim 25, wherein the patient is administered at least 8 subsequent doses.

28. The method of any one of the preceding claims, wherein the administration of the therapeutically effective dosing regimen results in reduction of the proptosis by about 1 to about 3 mm, about 1 to about 2 mm, about 2 to about 3 mm from baseline within 6 weeks of the first dose as measured by exophthalmometry or by MRI/CT.

29. The method of claim 28, wherein the proptosis is reduced by about 2-3 mm from baseline within 6 weeks of the first dose as measured by exophthalmometry or by MRI/CT.

30. The method of any one the preceding claims, wherein the patient does not have hearing impairment, ototoxic changes in audiometry, or hyperglycemic levels in the patient during the treatment.

31. The method of any one the preceding claims, wherein the light chain comprises a variable region having the amino acid sequence of SEQ ID NO: 2 and the heavy chain comprises a variable region sequence having the amino acid sequence of SEQ ID NO: 3.

32. The method of any one the preceding claims, wherein the light chain comprises an amino acid sequence of SEQ ID NO: 11.

33. The method of any one the preceding claims, wherein the heavy chain comprises an amino acid sequence of SEQ ID NO: 10.

34. The method of any one the preceding claims, wherein the heavy chain comprises an amino acid sequence of SEQ ID NO: 10 and the light chain comprises an amino acid sequence of SEQ ID NO: 11.

35. The method of any one of the preceding claims, wherein the patient achieves an improvement in one or more parameters selected from: proptosis, CAS, extraocular muscle volume, orbital fat volume, manual measurement of lid retraction, Graves' Orbitopathy-Quality of Life (GO-QoL) combined score, GO-QoL activity subscale, GO-QoL appearance subscale, visual acuity, Gorman Subjective Diplopia Score, and EQ-5D-5L QoL questionnaire.

36. The method of claim 35, wherein the patient achieves an improvement in at least two, three, four or more parameters.


SEQUENCE LISTING (45 sequences)

SEQ ID NO: 1 - skipped

SEQ ID NO: 2 - protein, 113 aa, synthetic construct
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 3 - protein, 124 aa, synthetic construct
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSS

SEQ ID NO: 4 - protein, 16 aa, synthetic construct
RSSQSIVHSNVNTYLE

SEQ ID NO: 5 - protein, 7 aa, synthetic construct
KVSNRFS

SEQ ID NO: 6 - protein, 9 aa, synthetic construct
FQGSHVPPT

SEQ ID NO: 7 - protein, 5 aa, synthetic construct
SYWMH

SEQ ID NO: 8 - protein, 18 aa, synthetic construct
GEINPSNGRTNYNQKFQG

SEQ ID NO: 9 - protein, 15 aa, synthetic construct
GRPDYYGSSKWYFDV

SEQ ID NO: 10 - protein, 454 aa, synthetic construct
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 219 aa, synthetic construct
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 6 aa, synthetic construct
GGGGSN

SEQ ID NO: 13 - protein, 5 aa, synthetic construct
GGGGA

SEQ ID NO: 14 - protein, 453 aa, synthetic construct
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 219 aa, synthetic construct; identical to SEQ ID NO: 11

SEQ ID NO: 16 - protein, 113 aa, synthetic construct
DIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIKR

SEQ ID NO: 17 - protein, 117 aa, synthetic construct [sequence omitted]

SEQ ID NO: 18 - protein, 20 aa, synthetic construct
RSSQSIVHSNGNTYLQWYLQ

SEQ ID NO: 19 - protein, 7 aa, synthetic construct
KVSNRLY

SEQ ID NO: 20 - protein, 9 aa, synthetic construct
FQGSHVPWT

SEQ ID NO: 21 - protein, 6 aa, synthetic construct
GGYLWN

SEQ ID NO: 22 - protein, 16 aa, synthetic construct
YISYDGTNNYKPSLKD

SEQ ID NO: 23 - protein, 8 aa, synthetic construct
YGRVFFDY

SEQ ID NO: 24 - protein, 446 aa, synthetic construct
QVQLQESGPGLVKPSETLSLTCTVSGYSITGGYLWNWIRQPPGKGLEWIGYISYDGTNNYKPSLKDRVTISRDTSKNQFSLKLSSVTAADTAVYYCARYGRVFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 219 aa, synthetic construct
DIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 18 aa, synthetic construct
GSTSGSGKPGSGEGSTKG

SEQ ID NO: 27 - protein, 11 aa, synthetic construct
QSIVHSNVNTY

SEQ ID NO: 28 - skipped

SEQ ID NO: 29 - protein, 8 aa, synthetic construct
GYTFTSYW

SEQ ID NO: 30 - protein, 8 aa, synthetic construct
INPSNGRT

SEQ ID NO: 31 - protein, 17 aa, synthetic construct
ARGRPDYYGSSKWYFDV

SEQ ID NO: 32 - protein, 7 aa, synthetic construct
GYTFTSY

SEQ ID NO: 33 - protein, 6 aa, synthetic construct
NPSNGR

SEQ ID NO: 34 - protein, 8 aa, synthetic construct
YKVSNRFS

SEQ ID NO: 35 - protein, 13 aa, synthetic construct
KASGYTFTSYWMH

SEQ ID NO: 36 - protein, 10 aa, synthetic construct
EINPSNGRTN

SEQ ID NO: 37 - protein, 11 aa, synthetic construct
QSIVHSNGNTY

SEQ ID NO: 38 - protein, 9 aa, synthetic construct
GYSITGGYL

SEQ ID NO: 39 - protein, 7 aa, synthetic construct
ISYDGTN

SEQ ID NO: 40 - protein, 10 aa, synthetic construct
ARYGRVFFDY

SEQ ID NO: 41 - protein, 8 aa, synthetic construct
GYSITGGY

SEQ ID NO: 42 - protein, 5 aa, synthetic construct
SYDGT

SEQ ID NO: 43 - protein, 8 aa, synthetic construct
YKVSNRLY

SEQ ID NO: 44 - protein, 14 aa, synthetic construct
TVSGYSITGGYLWN

SEQ ID NO: 45 - protein, 9 aa, synthetic construct
YISYDGTNN